(19) United States
(12) Patent Application Publication
(10) Pub. No.: US 2024/0000958 A1
(43) Pub. Date: Jan. 4, 2024

(54) NOVEL CARRIERS AND CONJUGATION METHODS

(71) Applicant: GLAXOSMITHKLINE BIOLOGICALS SA, Rixensart (BE)

(72) Inventors: Francesca Micoli, Rixensart (BE); Roberta Di Benedetto, Rixensart (BE); Allan Saul, Rixensart (BE)

(21) Appl. No.: 18/252,783

(22) PCT Filed: Nov. 12, 2021
(86) PCT No.: PCT/EP2021/081566

§ 371 Date: May 12, 2023

(30) Foreign Application Priority Data
Nov. 13, 2020 (EP) 20207547.9

(51) Int. Cl. A61K 47/64; A61K 39/09; A61P 31/04

(52) U.S. Cl. CPC A61K 47/646; A61K 39/092; A61P 31/04; A61K 2039/6037

(57) ABSTRACT

The present invention is directed to a polysaccharide conjugate comprising or consisting of a one or more polysaccharide conjugated to a carrier polypeptide, wherein the carrier polypeptide is selected from the group consisting of (a) a Streptococcus pyogenes SpyAD (Spy0269, GAS40), a Streptococcus pyogenes SpyCEP (Spy0416, GAS57), or Streptococcus pyogenes SLO (Spy0167, GAS25); (b) CRM₁₉₇; or (c) a variant, fragment and/or fusion of (a) or (b), improved conjugation methods, and uses of said conjugates for preventing or treating disease.

### FIELD OF THE INVENTION

The present invention relates to the use of antigens from Streptococcus pyogenes (Group A Streptococcus) for use as carrier proteins, together with improved conjugation methods, and uses of said conjugates for preventing and/or treating disease.

### BACKGROUND OF THE INVENTION

Group A Streptococcus (GAS) causes a diverse spectrum of diseases, from superficial infections (pharyngitis, skin infections, cellulitis) to severe invasive diseases (puerperal sepsis, necrotizing fasciitis, streptococcal toxic shock syndrome), with a high frequency of serious sequelae in low- and middle-income Countries (LMICs) (acute rheumatic fever, ARF; rheumatic heart disease, RHD, and glomerulonephritis) [1].

Pharyngitis is the most frequent symptomatic GAS infection in children across the world, with more than 400 million cases estimated annually [2] and an important driver of antibiotic use [3] that can ultimately result in increased antimicrobial resistance, a growing public health crisis [4]. Pharyngitis could lead to RHD, which is a chronic inflammatory heart valve condition representing the main global burden of GAS. In 2015, 319 thousand deaths due to RHD were estimated, with >33 million RHD cases and 10 million disability-adjusted life-years (DALYs) lost [5]. Vaccination is the most practical strategy to reduce global GAS associated disease burden in the long term. However, no commercial vaccine is still available against this pathogen [6].

Group A Carbohydrate (GAC) is a surface polysaccharide comprising of a polyrhamnose backbone with alternating N-acetylglucosamine (GlcNAc) at the side chain. It represents an attractive vaccine candidate as it is highly conserved and expressed across GAS strains. Indeed, one of the major obstacles for vaccine strategy development is represented by GAS serotype diversity related to other non-carbohydrate antigens [7].

Conjugation of polysaccharides (PS) to appropriate carrier proteins is a common procedure for improving their immunogenicity [8]. PS are typical T-cell independent antigens naturally containing only B-cell epitopes, and lacking T-cell epitopes. Covalent conjugation to a protein as a source of T-cell epitopes, converts the PS into a T-dependent antigen, with enhanced memory response, class-switching, and antibody production in infants [9, 10].

It has been reported that human anti-GAC sera successfully promoted phagocytosis of several GAS strains [11], while mice immunized with GAC conjugated to tetanus toxoid (TT) or CRM₁₉₇ carrier proteins were protected against GAS challenges [12, 13]. Moreover, an inverse relationship between high anti-GAC antibody titers and the presence of GAS in the throat of Mexican children was evidenced [13].

Conserved protein antigens are also in vaccine development against GAS. In particular, Streptolysin O (SLO), SpyAD and SpyCEP were identified as promising vaccine candidates through a reverse vaccinology approach [14]. SLO has been shown to be a key virulence factor of GAS by preventing internalization of the bacteria into lysosomes where they can be destroyed [15]. Moreover, SLO promotes GAS resistance to phagocytic clearance by neutrophils, facilitating GAS escape from innate immune killing, and an inactivated SLO demonstrated to be protective in a murine model against GAS challenge [16]. SpyAD is a surface-exposed adhesin that mediates GAS interaction with host cells. Moreover, deletion of SpyAD gene in a GAS strain led to an impaired capacity of the knockout mutant to properly divide, suggesting also an important role in bacterial division [17]. Finally, SpyCEP is a multi-domain proteinase, with a catalytic domain responsible for the interleukin (IL)-8 and other chemokines cleavage. Cleavage of IL-8 represents a mechanism of immune evasion, preventing IL-8 C-terminus-mediated endothelial translocation and subsequent recruitment of neutrophils [18, 19].

These three protein antigens are highly conserved and prevalent in clinical collections and, together with GAC, could virtually cover all GAS clinical isolates [20]. Thus, the formulation of a multicomponent vaccine composed of recombinant SLO, SpyAD and SpyCEP with GAC-CRM₁₉₇ conjugate has been proposed [6].

Since the serious sequelae of GAS primarily affect LMICs there is a need to reduce the cost of production as much as possible, to make the vaccine economically viable.

### DETAILED DESCRIPTION OF THE INVENTION

Here, the possibility to use one of the GAS proteins with dual role of antigen and carrier for GAC was tested, aiming to reduce the complexity of the final vaccine formulation. CRM₁₉₇ is one of the few carrier proteins currently used in licensed glycoconjugate vaccines against bacterial infections. For this reason, there is increased concern that pre-exposure or co-exposure to this carrier could lead to immune interference and reduction of the anti-carbohydrate immune response [9], thus driving to the need of identifying alternative carrier proteins [21, 22]. The present inventors surprisingly found that each of SLO, SpyAD and SpyCEP could be used as carrier protein for PS.

Chemical conjugation of a PS to a carrier protein is a complex process that can result in the lack of reproducibility and consistency if not performed under robust conditions. Although CRM₁₉₇ is a well-known carrier protein with well-established conjugation conditions, the present inventors surprisingly found, using a Design of Experiment (DoE) approach, that the robustness and yield of linkage of PS such as GAC to CRM₁₉₇ and GAS antigens could be improved.

Accordingly, a first aspect of the invention provides a polysaccharide conjugate comprising or consisting of one or more polysaccharide conjugated to a carrier polypeptide, wherein the carrier polypeptide is:
(a) a Streptococcus pyogenes SpyAD (Spy0269, GAS40), a Streptococcus pyogenes SpyCEP (Spy0416, GAS57), or a Streptococcus pyogenes SLO (Spy0167, GAS25);
(b) CRM₁₉₇; or
(c) a fragment, variant or fusion of (a) or (b).

By using GAS antigens as carrier proteins, an alternative to CRM₁₉₇ is provided, removing concerns that pre-exposure or co-exposure to this carrier could lead to immune interference and reduction of the anti-carbohydrate immune response. Moreover, use of GAS antigens as carrier polypeptides potentially allows CRM₁₉₇ to be removed from the proposed multicomponent vaccine formulation of recombinant SLO, SpyAD and SpyCEP with GAC-CRM₁₉₇ conjugate. This simplification would reduce the cost of production making the vaccine more economically viable, particularly in LMICs where profit margins are narrow. In the event that CRM₁₉₇ is retained in a vaccine formulation, the increased yield and robustness provided by the presently-disclosed conjugation method will contribute to lower production costs and, therefore, improve commercial viability in LMICs.

Accordingly, in one embodiment the polysaccharide conjugate is produced according to the method of the tenth aspect of the invention (described below).

Alternatively or additionally, the carrier polypeptide is:
(a) selected from the group consisting of a Streptococcus pyogenes SpyAD (Spy0269, GAS40), a Streptococcus pyogenes SpyCEP (Spy0416, GAS57), and Streptococcus pyogenes SLO (Spy0167, GAS25), or
(b) a variant, fragment and/or fusion of (a).

By ‘one or more polysaccharide conjugated to a carrier polypeptide’ we mean or include that (a) one or more polysaccharide molecule may be conjugated to the carrier polypeptide; and/or (b) that one or more molecular species of polysaccharide may be conjugated to the carrier polypeptide (e.g., different polysaccharides of the same genus, species or strain, or polysaccharides from different genera, species or strains).

By ‘SpyAD (Spy0269, GAS40)’ we mean or include a polypeptide comprising or consisting of an amino acid sequence according to SEQ ID NO: 1, SEQ ID NO:2 or NCBI reference sequence WP_010921884.1.

native sequence from GAS parent stain SF370
minus N-terminal exclusion domain
[SEQ ID NO: 1]
MSVGVSHQVKADDRASGETKASNTHDDSLPKPETIQEAKATIDAVEKTLS
QQKAELTELATALTKTTAEINHLKEQQDNEQKALTSAQEIYTNTLASSEE
TLLAQGAEHQRELTATETELHNAQADQHSKETALSEQKASISAETTRAQD
LVEQVKTSEQNIAKLNAMISNPDAITKAAQTANDNTKALSSELEKAKADL
ENQKAKVKKQLTEELAAQKAALAEKEAELSRLKSSAPSTQDSIVGNNTMK
APQGYPLEELKKLEASGYIGSASYNNYYKEHADQIIAKASPGNQLNQYQD
IPADRNRFVDPDNLTPEVQNELAQFAAHMINSVRRQLGLPPVTVTAGSQE
FARLLSTSYKKTHGNTRPSFVYGQPGVSGHYGVGPHDKTIIEDSAGASGL
IRNDDNMYENIGAFNDVHTVNGIKRGIYDSIKYMLFTDHLHGNTYGHAIN
FLRVDKHNPNAPVYLGFSTSNVGSLNEHFVMFPESNIANHQRENKTPIKA
VGSTKDYAQRVGTVSDTIAAIKGKVSSLENRLSAIHQEADIMAAQAKVSQ
LQGKLASTLKQSDSLNLQVRQLNDTKGSLRTELLAAKAKQAQLEATRDQS
LAKLASLKAALHQTEALAEQAAARVTALVAKKAHLQYLRDFKLNPNRLQV
IRERIDNTKQDLAKTTSSLLNAQEALAALQAKQSSLEATIATTEHQLTLL
KTLANEKEYRHLDEDIATVPDLQVAPPLTGVKPLSYSKIDTTPLVQEMVK
ETKQLLEASARLAAENTSLVAEALVGQTSEMVASNAIVSKITSSITQPSS
KTSYGSGSSTTSNLISDVDESTQR*
native sequence from GAS parent stain SF370
with N-terminal exclusion domain (exclusion
domain indicated by underlining)
[SEQ ID NO: 2]
MDLEQTKPNQVKQKIALTSTIALLSASVGVSHQVKADDRASGETKASNTH
DDSLPKPETIQEAKATIDAVEKTLSQQKAELTELATALTKTTAEINHLKE
QQDNEQKALTSAQEIYTNTLASSEETLLAQGAEHQRELTATETELHNAQA
DQHSKETALSEQKASISAETTRAQDLVEQVKTSEQNIAKLNAMISNPDAI
TKAAQTANDNTKALSSELEKAKADLENQKAKVKKQLTEELAAQKAALAEK
EAELSRLKSSAPSTQDSIVGNNTMKAPQGYPLEELKKLEASGYIGSASYN
NYYKEHADQIIAKASPGNOLNQYQDIPADRNRFVDPDNLTPEVQNELAQF
AAHMINSVRRQLGLPPVTVTAGSQEFARLLSTSYKKTHGNTRPSFVYGQP
GVSGHYGVGPHDKTIIEDSAGASGLIRNDDNMYENIGAFNDVHTVNGIKR
GIYDSIKYMLETDHLHGNTYGHAINFLRVDKHNPNAPVYLGFSTSNVGSL
NEHFVMFPESNIANHQRENKTPIKAVGSTKDYAQRVGTVSDTIAAIKGKV
SSLENRLSAIHQEADIMAAQAKVSQLQGKLASTLKQSDSLNLQVRQLNDT
KGSLRTELLAAKAKQAQLEATRDQSLAKLASLKAALHQTEALAEQAAARV
TALVAKKAHLQYLRDFKLNPNRLQVIRERIDNTKQDLAKTTSSLLNAQEA
LAALQAKQSSLEATIATTEHQLTLLKTLANEKEYRHLDEDIATVPDLQVA
PPLTGVKPLSYSKIDTTPLVQEMVKETKQLLEASARLAAENTSLVAEALV
GQTSEMVASNAIVSKITSSITQPSSKTSYGSGSSTTSNLISDVDESTQRA
LKAGVVMLAAVGLTGFRFRKESK

By ‘SpyCEP (Spy0416, GAS57)’ we mean or include a polypeptide comprising or consisting of an amino acid sequence according to SEQ ID NO: 3, SEQ ID NO: 4 or NCBI reference sequence WP 010921938.1.

SpyCEP detoxified double mutant
[SEQ ID NO: 3]
MADELSTMSEPTITNHAQQQAQHLTNTELSSAESKSQDTSQITLKTNREK
EQSQDLVSEPTTTELADTDAASMANTGSDATQKSASLPPVNTDVHDWVKT
KGAWDKGYKGQGKVVAVIATGIDPAHQSMRISDVSTAKVKSKEDMLARQK
AAGINYGSWINDKVVFAHNYVENSDNIKENQFEDEDEDWENFEFDAEAEP
KAIKKHKIYRPQSTQAPKETVIKTEETDGSHDIDWTQTDDDTKYESHGMH
VTGIVAGNSKEAAATGERFLGIAPEAQVMEMRVFANDIMGSAESLFIKAI
EDAVALGADVINLSLGTANGAQLSGSKPLMEAIEKAKKAGVSVVVAAGNE
RVYGSDHDDPLATNPDYGLVGSPSTGRTPTSVAAINSKWVIQRLMTVKEL
ENRADLNHGKAIYSESVDEKDIKDSLGYDKSHQFAYVKESTDAGYNAQDV
KGKIALIERDPNKTYDEMIALAKKHGALGVLIENNKPGQSNRSMRLTANG
MGIPSAFISHEFGKAMSQLNGNGTGSLEFDSVVSKAPSQKGNEMNHFSNW
GLTSDGYLKPDITAPGGDIYSTYNDNHYGSQTGTAMASPQIAGASLLVKQ
YLEKTQPNLPKEKIADIVKNLLMSNAQIHVNPETKTTTSPRQQGAGLLNI
DGAVTSGLYVTGKDNYGSISLGNITDTMTFDVTVHNLSNKDKTLRYDTEL
LTDHVDPQKGRFTLTSHSLKTYQGGEVTVPANGKVTVRVTMDVSQFTKEL
TKQMPNGYYLEGEVRERDSQDDQLNRVNIPFVGFKGQFENLAVAEESIYR
LKSQGKTGFYFDESGPKDDIYVGKHFTGLVTLGSETNVSTKTISDNGLHT
LGTEKNADGKFILEKNAQGNPVLAISPNGDNNQDFAAFKGVELRKYQGLK
ASVYHASDKEHKNPLWVSPESEKGDKNENSDIRFAKSTTLLGTAFSGKSL
TGAELPDGHYHYVVSYYPDVVGAKRQEMTEDMILDRQKPVLSQATFDPET
NRFKPEPLKDRGLAGVRKDSVFYLERKDNKPYTVTINDSYKYVSVEDNKT
FVERQADGSFILPLDKAKLGDFYYMVEDFAGNVAIAKLGDHLPQTLGKTP
IKLKLTDGNYQTKETLKDNLEMTQSDTGLVTNQAQLAVVHRNQPQSQLTK
MNQDFFISPNEDGNKDFVAFKGLKNNVYNDLTVNVYAKDDHQKQTPIWSS
QAGASVSAIESTAWYGITARGSKVMPGDYQYVVTYRDEHGKEHQKQYTIS
VNDKKPMITQGREDTINGVDHFTPDKTKALDSSGIVREEVFYLAKKNGRK
EDVTEGKDGITVSDNKVYIPKNPDGSYTISKRDGVTLSDYYYLVEDRAGN
VSFATLRDLKAVGKDKAVVNFGLDLPVPEDKQIVNETYLVRDADGKPIEN
LEYYNNSGNSLILPYGKYTVELLTYDTNAAKLESDKIVSFTLSADNNFQQ
VTFKITMLATSQITAHFDHLLPEGSRVSLKTAQDQLIPLEQSLYVPKAYG
KTVQEGTYEVVVSLPKGYRIEGNTKVNTLPNEVHELSLRLVKVGDASDST
GDHKVMSKNNSQALTASATPTKSTTSATAKA*
full-length, native SpyCEP from GAS
strain SF370
[SEQ ID NO: 4]
MEKKQRFSLRKYKSGTFSVLIGSVFLVMTTTVAADELSTMSEPTITNHAQ
QQAQHLINTELSSAESKSQDTSQITLKTNREKEQSQDLVSEPTTTELADT
DAASMANTGSDATQKSASLPPVNTDVHDWVKTKGAWDKGYKGQGKVVAVI
DTGIDPAHQSMRISDVSTAKVKSKEDMLARQKAAGINYGSWINDKVVEAH
NYVENSDNIKENQFEDEDEDWENFEFDAEAEPKAIKKHKIYRPQSTQAPK
ETVIKTEETDGSHDIDWTQTDDDTKYESHGMHVTGIVAGNSKEAAATGER
FLGIAPEAQVMEMRVFANDIMGSAESLFIKAIEDAVALGADVINLSLGTA
NGAQLSGSKPLMEAIEKAKKAGVSVVVAAGNERVYGSDHDDPLATNPDYG
LVGSPSTGRTPTSVAAINSKWVIQRLMTVKELENRADLNHGKAIYSESVD
EKDIKDSLGYDKSHQFAYVKESTDAGYNAQDVKGKIALIERDPNKTYDEM
IALAKKHGALGVLIFNNKPGQSNRSMRLTANGMGIPSAFISHEFGKAMSQ
LNGNGTGSLEFDSVVSKAPSQKGNEMNHFSNWGLTSDGYLKPDITAPGGD
IYSTYNDNHYGSQTGTSMASPQIAGASLLVKQYLEKTQPNLPKEKIADIV
KNLLMSNAQIHVNPETKTTTSPRQQGAGLLNIDGAVTSGLYVTGKDNYGS
ISLGNITDTMTFDVTVHNLSNKDKTLRYDTELLTDHVDPQKGRFTLTSHS
LKTYQGGEVTVPANGKVTVRVTMDVSQFTKELTKOMPNGYYLEGFVRFRD
SQDDQLNRVNIPFVGFKGQFENLAVAEESIYRLKSQGKTGFYFDESGPKD
DIYVGKHFTGLVTLGSETNVSTKTISDNGLHTLGTFKNADGKFILEKNAQ
GNPVLAISPNGDNNQDFAAFKGVELRKYQGLKASVYHASDKEHKNPLWVS
PESEKGDKNENSDIRFAKSTTLLGTAFSGKSLTGAELPDGHYHYVVSYYP
DVVGAKRQEMTEDMILDRQKPVLSQATEDPETNRFKPEPLKDRGLAGVRK
DSVFYLERKDNKPYTVTINDSYKYVSVEDNKTFVERQADGSFILPLDKAK
LGDFYYMVEDFAGNVAIAKLGDHLPQTLGKTPIKLKLTDGNYQTKETLKD
NLEMTQSDTGLVTNQAQLAVVHRNQPQSQLTKMNQDFFISPNEDGNKDFV
AFKGLKNNVYNDLTVNVYAKDDHQKQTPIWSSQAGASVSAIESTAWYGIT
ARGSKVMPGDYQYVVTYRDEHGKEHQKQYTISVNDKKPMITQGREDTING
VDHFTPDKTKALDSSGIVREEVFYLAKKNGRKFDVTEGKDGITVSDNKVY
IPKNPDGSYTISKRDGVTLSDYYYLVEDRAGNVSFATLRDLKAVGKDKAV
VNFGLDLPVPEDKQIVNFTYLVRDADGKPIENLEYYNNSGNSLILPYGKY
TVELLTYDTNAAKLESDKIVSFTLSADNNFQQVTFKITMLATSQITAHFD
HLLPEGSRVSLKTAQDQLIPLEQSLYVPKAYGKTVQEGTYEVVVSLPKGY
RIEGNTKVNTLPNEVHELSLRLVKVGDASDSTGDHKVMSKNNSQALTASA
TPTKSTTSATAKALPSTGEKMGLKLRIVGLVLLGLTCVFSRKKSTKD

By ‘SLO (Spy0167, GAS25)’ we mean or include a polypeptide comprising or consisting of an amino acid sequence according to SEQ ID NO: 5, SEQ ID NO: 6 or NCBI reference sequence WP_010921831.1.

SLO detoxified double mutant
[SEQ ID NO: 5]
MASESNKQNTASTETTTTNEQPKPESSELTTEKAGQKTDDMLNSNDMIKL
APKEMPLESAEKEEKKSEDKKKSEEDHTEEINDKIYSLNYNELEVLAKNG
ETIENFVPKEGVKKADKFIVIERKKKNINTTPVDISIIDSVTDRTYPAAL
QLANKGFTENKPDAVVTKRNPQKIHIDLPGMGDKATVEVNDPTYANVSTA
IDNLVNQWHDNYSGGNTLPARTQYTESMVYSKSQIEAALNVNSKILDGTL
GIDFKSISKGEKKVMIAAYKQIFYTVSANLPNNPADVEDKSVTFKELQRK
GVSNEAPPLEVSNVAYGRTVFVKLETSSKSNDVEAAFSAALKGTDVKING
KYSDILENSSFTAVVLGGDAAEHNKVVTKDEDVIRNVIKDNATESRKNLA
YPISYTSVFLKNNKIAGVNNRTEYVETTSTEYTSGKINLSHQGAYVAQYE
ILWDEINYDDKGKEVITKRRWDNNWYSKTSPFSTVIPLGANSRNIRIMAR
ECTGLAFEWWRKVIDERDVKLSKEINVNISGSTLSPYGSITYK*
full-length, native SLO from GAS
strain SF370
[SEQ ID NO: 6]
MSNKKTFKKYSRVAGLLTAALIIGNLVTANAESNKQNTASTETTTTNEQP
KPESSELTTEKAGQKTDDMLNSNDMIKLAPKEMPLESAEKEEKKSEDKKK
SEEDHTEEINDKIYSLNYNELEVLAKNGETIENFVPKEGVKKADKFIVIE
RKKKNINTTPVDISIIDSVTDRTYPAALQLANKGFTENKPDAVVTKRNPQ
KIHIDLPGMGDKATVEVNDPTYANVSTAIDNLVNQWHDNYSGGNTLPART
QYTESMVYSKSQIEAALNVNSKILDGTLGIDEKSISKGEKKVMIAAYKQI
FYTVSANLPNNPADVEDKSVTEKELQRKGVSNEAPPLFVSNVAYGRTVFV
KLETSSKSNDVEAAFSAALKGTDVKINGKYSDILENSSFTAVVLGGDAAE
HNKVVTKDEDVIRNVIKDNATFSRKNPAYPISYTSVELKNNKIAGVNNRT
EYVETTSTEYTSGKINLSHQGAYVAQYEILWDEINYDDKGKEVITKRRWD
NNWYSKTSPFSTVIPLGANSRNIRIMARECTGLAWEWWRKVIDERDVKLS
KEINVNISGSTLSPYGSITYK

Alternatively or additionally, the carrier polypeptide is:
(a) CRM₁₉₇, or
(b) a variant, fragment and/or fusion of (a).

By ‘CRM₁₉₇’ we mean or include a polypeptide comprising or consisting of an amino acid sequence according to SEQ ID NO: 7.

CRM₁₉₇
[SEQ ID NO: 7]
MGADDVVDSSKSFVMENFSSYHGTKPGYVDSIQKGIQKPKSGTQGNYDDD
WKEFYSTDNKYDAAGYSVDNENPLSGKAGGVVKVTYPGLTKVLALKVDNA
ETIKKELGLSLTEPLMEQVGTEEFIKREGDGASRVVLSLPFAEGSSSVEY
INNWEQAKALSVELEINFETRGKRGQDAMYEYMAQACAGNRVRRSVGSSL
SCINLDWDVIRDKTKTKIESLKEHGPIKNKMSESPNKTVSEEKAKQYLEE
FHQTALEHPELSELKTVTGTNPVFAGANYAAWAVNVAQVIDSETADNLEK
TTAALSILPGIGSVMGIADGAVHHNTEEIVAQSIALSSLMVAQAIPLVGE
LVDIGFAAYNEVESIINLFQVVHNSYNRPAYSPGHKTQPFLHDGYAVSWN
TVEDSIIRTGFQGESGHDIKITAENTPLPIAGVLLPTIPGKLDVNKSKTH
ISVNGRKIRMRCRAIDGDVTFCRPKSPVYVGNGVHANLHVAFHRSSSEKI
HSNEISSDSIGVLGYQKTVDHTKVNSKLSLFFEIKS

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

Thus, when an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

By ‘isolated’ we mean that the feature (e.g., the polypeptide) of the invention is provided in a context other than that in which it may be found naturally. One of skill in the art would understand that ‘isolated’ means altered ‘by the hand of man’ from its natural state, i.e., if it occurs in nature, it has been changed or removed from its original environment, or both. For example, a polynucleotide or a polypeptide naturally present in a living organism is not ‘isolated’ when in such living organism, but the same polynucleotide or polypeptide separated from the coexisting materials of its natural state is ‘isolated’ as the term is used in this disclosure. Further, a polynucleotide or polypeptide that is introduced into an organism by transformation, genetic manipulation or by any other recombinant method would be understood to be ‘isolated’ even if it is still present in said organism, which organism may be living or non-living, except where such transformation, genetic manipulation or other recombinant method produces an organism that is otherwise indistinguishable from the naturally-occurring organism.

By ‘polypeptide’ we mean or include polypeptides and proteins.

By ‘variant’ of the polypeptide we include insertions, deletions and/or substitutions, either conservative or non-conservative. In particular, the variant polypeptide may be a non-naturally occurring variant (i.e., does not, or is not known to, occur in nature).

‘Sequence identity’ or ‘identity’ can be determined by the Smith Waterman homology search algorithm as implemented in the MPSRCH program (Oxford Molecular), using an affine gap search with parameters gap open penalty=12 and gap extension penalty=1, orby the Needleman-Wunsch global alignment algorithm (see e.g. Rubin (2000) Pediatric. Clin. North Am. 47:269-285), using default parameters (e.g. with Gap opening penalty=10.0, and with Gap extension penalty=0.5, using the EBLOSUM62 scoring matrix). This algorithm is conveniently implemented in the needle tool in the EMBOSS package. Unless specified otherwise, where the application refers to sequence identity to a particular reference sequence, the identity is intended to be calculated over the entire length of that reference sequence. Alternatively, percent identity can be determined by methods well known in the art, for example using the LALIGN program (Huang and Miller, Adv. Appl. Math. (1991) 12:337-357, the disclosures of which are incorporated herein by reference) at the ExPASy facility website www.ch.embnet.org/software/LALIGN_form.html using as parameters the global alignment option, scoring matrix BLOSUM62, opening gap penalty −14, extending gap penalty −4. Alternatively, the percent sequence identity between two polypeptides may be determined using suitable computer programs, for example AlignX, Vector NTI Advance 10 (from Invitrogen Corporation) or the GAP program (from the University of Wisconsin Genetic Computing Group).

It will be appreciated that percent identity is calculated in relation to polymers (e.g., polypeptide or polynucleotide) whose sequence has been aligned.

Fragments and variants may be made using the methods of protein engineering and site-directed mutagenesis well known in the art (for example, see Molecular Cloning: a Laboratory Manual, 3rd edition, Sambrook & Russell, 2001, Cold Spring Harbor Laboratory Press, the disclosures of which are incorporated herein by reference).

Alternatively or additionally, the carrier polypeptide is:
(a) a Streptococcus pyogenes SpyAD (Spy0269); or
(b) a variant, fragment and/or fusion of a Streptococcus pyogenes SpyAD (Spy0269).

Alternatively or additionally, the Streptococcus pyogenes SpyAD (Spy0269) comprises or consists of:
(i) the amino acid sequence of SEQ ID NO: 1 or SEQ ID NO: 2;
(ii) an amino acid sequence comprising from 1 to 10 single amino acid alterations compared to SEQ ID NO: 1 or SEQ ID NO: 2;
(iii) an amino acid sequence with at least 70% sequence identity with SEQ ID NO: 1 or SEQ ID NO: 2, for example, at least 80%, 85%, 90%, 95%, 96%, 97%, 98%, 99% or at least 99.5% identity with SEQ ID NO: 1 or SEQ ID NO: 2; and/or
(iv) a fragment of at least 10 consecutive amino acids from SEQ ID NO: 1 or SEQ ID NO: 2, for example, at least 20, 30, 40, 50, 60, 70, 80, 90, 100, 125, 150, 175, 200, 250, 275, 280, 290, 300, 310, 320, 330, 340, or 350 consecutive amino acids from SEQ ID NO: 1 or SEQ ID NO: 2.

Alternatively or additionally, the carrier polypeptide is:
(a) a Streptococcus pyogenes SpyCEP (Spy0416);
(b) a variant, fragment and/or fusion of a Streptococcus pyogenes SpyCEP (Spy0416).

Alternatively or additionally, the Streptococcus pyogenes SpyCEP (Spy0416) comprises or consists of:
(i) the amino acid sequence of SEQ ID NO: 3 or SEQ ID NO: 4;
(ii) an amino acid sequence comprising from 1 to 10 single amino acid alterations compared to SEQ ID NO: 3 or SEQ ID NO: 4;
(iii) an amino acid sequence with at least 70% sequence identity with SEQ ID NO: 3 or SEQ ID NO: 4, for example, at least 80%, 85%, 90%, 95%, 96%, 97%, 98%, 99% or at least 99.5% identity with SEQ ID NO: 3 or SEQ ID NO: 4; and/or
(iv) a fragment of at least 10 consecutive amino acids from SEQ ID NO: 3 or SEQ ID NO: 4, for example, at least 20, 30, 40, 50, 60, 70, 80, 90, 100, 125, 150, 175, 200, 250, 275, 280, 290, 300, 310, 320, 330, 340, 350, 500, 750, 1000, 1250, 1500, 1550, 1600, 1610, 1620, 1630, 1640, 1650 or 1660 consecutive amino acids from SEQ ID NO: 3 or SEQ ID NO: 4.

Alternatively or additionally, the carrier polypeptide is:
(a) a Streptococcus pyogenes Slo (Spy0167); or
(b) a variant, fragment and/or fusion of a Streptococcus pyogenes Slo (Spy0167).

Alternatively or additionally, the Streptococcus pyogenes Slo (Spy0167) comprises or consists of:
(i) the amino acid sequence of SEQ ID NO: 5 or SEQ ID NO: 6;
(ii) an amino acid sequence comprising from 1 to 10 single amino acid alterations compared to SEQ ID NO: 5 or SEQ ID NO: 6;
(iii) an amino acid sequence with at least 70% sequence identity with SEQ ID NO: 5 or SEQ ID NO: 6, for example, at least 80%, 85%, 90%, 95%, 96%, 97%, 98%, 99% or at least 99.5% identity with SEQ ID NO: 5 or SEQ ID NO: 6; and/or
(iv) a fragment of at least 10 consecutive amino acids from SEQ ID NO: 5 or SEQ ID NO: 6, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, 540, 550, 560 or 570 consecutive amino acids from SEQ ID NO: 5 or SEQ ID NO: 6.

Alternatively or additionally, the carrier polypeptide is:
(a) CRM197; or
(b) a variant, fragment and/or fusion of CRM197.

Alternatively or additionally, the CRM197 comprises or consists of:
(i) the amino acid sequence of SEQ ID NO: 7;
(ii) an amino acid sequence comprising from 1 to 10 single amino acid alterations compared to SEQ ID NO: 7;
(iii) an amino acid sequence with at least 70% sequence identity with SEQ ID NO: 7, for example, at least 80%, 85%, 90%, 95%, 96%, 97%, 98%, 99% or at least 99.5% identity with SEQ ID NO: 7; and/or
(iv) a fragment of at least 10 consecutive amino acids from SEQ ID NO: 7, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, or 535 consecutive amino acids from SEQ ID NO: 7.

Alternatively or additionally, the one or more polysaccharide is a microbial polysaccharide such as a bacterial polysaccharide, an archaea polysaccharide, a fungal polysaccharide, or a protist polysaccharide. Alternatively or additionally, the microbe is a pathogen, for example, a human pathogen.

Alternatively or additionally, the polysaccharide is from a mammalian cell, for example, a cancer cell. Where the polysaccharide is from a mammalian cancer cell, it is preferred that the polysaccharide is solely or predominantly expressed by the cancer cell. Preferably the mammalian cell is a human cell.

By ‘predominantly expressed’ we mean or include (a) that the polysaccharide is expressed (in particular, expressed in a manner accessible by host antibodies when the cell is intact [e.g., when the cell has not apoptosed]) at least 50% less w/w on host non-cancer cells than on the host tumour cell, for example, at least 60%, 70%, 80%, 90% 95%, 98%, 99%, or least 99.9% less than on the host cancer cell (b) and/or that the polysaccharide is expressed on at least 50% or fewer host non-cancer cells, for example, at least 60%, 70%, 80%, 90% 95%, 98%, 99%, or least 99.9% fewer host non-cancer cells.

Alternatively or additionally, the one or more polysaccharide is surface-expressed. By ‘the one or more polysaccharide is surface-expressed’ we mean or include that the polysaccharide is expressed on the cell surface of its originator cell (e.g., if the polysaccharide is of bacterial origin, that it is expressed by the bacteria on its cell surface), e.g., in a manner accessible to host antibodies.

Alternatively or additionally, the one or more polysaccharide is a bacterial polysaccharide, for example, a polysaccharide (such as a capsular polysaccharide or lipopolysaccharide) of a bacterium selected from the group consisting of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

Alternatively or additionally, the one or more polysaccharide comprises or consists of deoxy sugar monomers, for example, deoxy sugars selected from the group consisting of rhamnose (6-deoxy-L-mannose), fuculose (6-deoxy-L-tagatose), or fucose (6-deoxy-L-galactose).

Alternatively or additionally, the one or more polysaccharide comprises side chain, for example, side chain comprises or consisting of N-acetylglucosamine (GlcNAc), however, the one or more polysaccharide may alternatively consist of polysaccharide without side chains (so-called backbone polysaccharide).

By ‘polysaccharide’ we mean or include any linear or branched polymer consisting of monosaccharide residues, usually linked by glycosidic linkages, and thus includes oligosaccharides. The polysaccharide may contain between 2 and 50 monosaccharide unites, more preferably between 6 and 30 monosaccharide units.

By a fragment of a polysaccharide we mean polysaccharides that are truncated compared to the wild-type polysaccharide (e.g., have an average [mean] number of monosaccharide units compared to the wild-type polysaccharide). Polysaccharide truncation can be achieved by any suitable means known in the art such as chemical digestion, in vitro polysaccharide synthesis of polysaccharide with fewer monosaccharide units than wild-type, or genetic modification of polysaccharide producing strains.

By a variant of a polysaccharide we mean or include that one or more chemical group of the polysaccharide backbone and/or side chain(s) is modified compared to wild-type polysaccharide. Polysaccharide modification can be achieved by any suitable means known in the art such as chemical reaction or genetic modification of polysaccharide producing strains.

By a fusion of a polysaccharide we mean or include that the polysaccharide, fragment or variant thereof is covalently or ionically bonded or otherwise fused to one or more other component. The one or more other component may be a polysaccharide of a different molecular species (e.g., from a different genus, species or strain) or a fragment or variant thereof.

It will be appreciated that the or each carrier protein may have single or multiple polysaccharides conjugated to it. Hence, alternatively or additionally, an average of 1, 1.5 2, 2.5 3, 3.5 4, 4.5, 5, 6, 7, 8, 9 10, 11, 12, 13, 14, 15, 16, 17, 18, 19, or 20 polysaccharide molecules are conjugated to the carrier polypeptide.

By ‘an average of X polysaccharide molecules are conjugated to the carrier polypeptide’ (wherein X is a number between 1 and 15) we mean or include that an average (mean) of X polysaccharides are conjugated to the or each carrier polypeptide.

Where multiple polysaccharides are conjugated to the or each carrier protein, it will be appreciated that each polysaccharide may be of an identical species, e.g., to increase the potency of the immune response induced. On the other hand, a mixture of polysaccharide species may be conjugated to the or each carrier protein, e.g., to increase the valence of immune response induced (i.e., to broaden species/strain coverage or target multiple antigens on a single species/strain). Hence, alternatively or additionally, the one or more polysaccharide comprises or consists of:
I. a single molecular species; or
II. a mixture of molecular species, for example, 2, 3, 4, 5 6, 7, 8, 9, 10, 11, 12, 13, 14, 15, 16, 17, 18, 19, or 20 molecular species.

By ‘molecular species’ we mean or include polysaccharides comprising or consisting of (a) chemically identical sugar backbones, (b) chemically identical sugar backbones and side chains, or (c) chemically identical sugar backbones, chemically identical side chains, and identical sugar backbone length and side chain length, (d) wholly identical polysaccharide molecules.

By ‘mixture of molecular species’ we mean or include the polysaccharide conjugated to carrier polypeptide comprises or consists of at least two different ‘molecular species’. Different molecular species may, for example, (a) have chemically different sugar backbones, (b) have chemically different sugar backbones and side chains, or (c) have chemically different sugar backbones, chemically different side chains, and different sugar backbone length and side chain length, (d) be wholly different polysaccharide molecules. The at least two different molecular species may be conjugated to the carrier polypeptide in equal ratio (e.g., where two species are conjugated a ratio of 1:1, where three species are conjugated a ratio of 1:1:1). Alternatively, one or more molecular species may be conjugated to the carrier polypeptide in unequal ratios, for example, where there are two molecular species, a ratio of 1.5:1, 2:1, 3:1, 4:1, 5:1, 6:1, 7:1, 8:1, 9:1 or 10:1, or where there are three molecular species, a ratio of 1.5:1:1, 2:1:1, 3:1:1, 4:1:1, 5:1:1, 6:1:1, 7:1:1, 8:1:1, 9:1:1 or 10:1:1. The ratios may have a tolerance of +/−5%, for example, +/−4%, +/−3%, +/−2%, +/−1%, +/−0.5%, +/−0.25% or +/−0.1%. In one embodiment, GAC is the first molecular species. In an alternative embodiment, GAC is the second or (where present) third molecular species.

The one or more polysaccharide may be conjugated to the carrier protein directly. Alternatively or additionally, the one or more polysaccharide is conjugated to the carrier protein via a linker. Any suitable conjugation reaction can be used, with any suitable linker where necessary.

Attachment of the polysaccharide to the carrier polypeptide is preferably via a —NH2 group, e.g., through the side chain(s) of a lysine residue(s) or arginine residue(s) in the carrier polypeptide. Where the polysaccharide has a free aldehyde group, this group can react with an amine in the carrier polypeptide to form a conjugate by reductive amination. Attachment to the carrier may also be via a —SH group, e.g., through the side chain(s) of a cysteine residue(s) in the carrier polypeptide. Alternatively the polysaccharide may be attached to the carrier protein via a linker molecule.

The polysaccharide will typically be activated or functionalised prior to conjugation. Activation may involve, for example, cyanylating reagents such as CDAP (1-cyano-4-dimethylamino pyridinium tetrafluoro borate). Other suitable techniques use carbodiimides, hydrazides, active esters, norborane, p-nitrobenzoic acid, N-hydroxysuccinimide, S-NHS, EDC, TSTU (see, e.g., the introduction to WO98/42721).

Direct linkages to the carrier polypeptide may comprise oxidation of the polysaccharide followed by reductive amination with the carrier polypeptide, as described in, for example, U.S. Pat. Nos. 4,761,283 and 4,356,170. Linkages via a linker group may be made using any known procedure, for example, the procedures described in U.S. Pat. Nos. 4,882,317 and 4,695,624. Typically, the linker is attached via an anomeric carbon of the polysaccharide. A preferred type of linkage is an adipic acid linker, which may be formed by coupling a free —NH2 group (e.g., introduced to a polysaccharide by amination) with adipic acid (using, for example, diimide activation), and then coupling a protein to the resulting saccharide-adipic acid intermediate (see, e.g., EP-B-0477508, Mol. Immunol, (1985) 22, 907-919, and EP-A-0208375). A similar preferred type of linkage is a glutaric acid linker, which may be formed by coupling a free —NH group with glutaric acid in the same way. Adipic and glutaric acid linkers may also be formed by direct coupling to the polysaccharide, i.e., without prior introduction of a free group, e.g., a free —NH group, to the polysaccharide, followed by coupling a protein to the resulting saccharide-adipic/glutaric acid intermediate. Another preferred type of linkage is a carbonyl linker, which may be formed by reaction of a free hydroxyl group of a modified polysaccharide with CDI (Bethell G. S. et al. (1979) J Biol Chem 254, 2572-4 and Hearn M. T. W. (1981) J. Chromatogr 218, 509-18); followed by reaction with a protein to form a carbamate linkage. Other linkers include β-propionamido (WO00/10599), nitrophenyl-ethylamine (Gever et al. (1979) Med Microbiol Immunol 165, 171-288), haloacyl halides (U.S. Pat. No. 4,057,685), glycosidic linkages (U.S. Pat. Nos. 4,673,574; 4,761,283; and 4,808,700), 6-aminocaproic acid (U.S. Pat. No. 4,459,286), N-succinimidyl-3-(2-pyridyldithio)-propionate (SPDP) (U.S. Pat. No. 5,204,098), adipic acid dihydrazide (ADH) (U.S. Pat. No. 4,965,338), C4 to C12 moieties (U.S. Pat. No. 4,663,160), etc. Carbodiimide condensation can also be used (WO2007/000343).

A bifunctional linker may be used to provide a first group for coupling to an amine group in the polysaccharide (e.g., introduced to the polysaccharide by amination) and a second group for coupling to the carrier (typically for coupling to an amine in the carrier). Alternatively, the first group is capable of direct coupling to the polysaccharide, i.e., without prior introduction of a group, e.g., an amine group, to the polysaccharide.

In some embodiments, the first group in the bifunctional linker is thus able to react with an amine group (—NH2) on the polysaccharide. This reaction will typically involve an electrophilic substitution of the amine's hydrogen. In other embodiments, the first group in the bifunctional linker is able to react directly with the polysaccharide. In both sets of embodiments, the second group in the bifunctional linker is typically able to react with an amine group on the carrier polypeptide. This reaction will again typically involve an electrophilic substitution of the amine.

Where the reactions with both the polysaccharide and the carrier protein involve amines then it is preferred to use a bifunctional linker. For example, a homobifunctional linker of the formula X-L-X, may be used where: the two X groups are the same as each other and can react with the amines; and where L is a linking moiety in the linker. Similarly, a heterobifunctional linker of the formula X-L-X may be used, where: the two X groups are different and can react with the amines; and where L is a linking moiety in the linker. A preferred X group is N-oxysuccinimide. L preferably has formula L′-L²-L′, where L′ is carbonyl. Preferred L² groups are straight chain alkyls with 1 to 110 carbon atoms (e.g., Ci, C2, C3, C4, C5, C6, C7, C8, C9, C10) e.g. —(CH₂)₄- or —(CH₂)₃-.

Other X groups for use in the bifunctional linkers described in the preceding paragraph are those which form esters when combined with HO-L-OH, such as norborane, p-nitrobenzoic acid, and sulfo-N-hydroxysuccinimide.

Further bifunctional linkers for use with the invention include acryloyl halides (e.g., chloride) and haloacylhalides.

Other bifunctional linkers of particular use are selected from the group consisting of: acryloyl halides, preferably chloride, disuccinimidyl glutarate, disuccinimidyl suberate and ethylene glycol bis[succinimidylsuccinate]. Other useful linkers are selected from the group consisting of: P-propionamido, nitrophenyl-ethylamine, haloacyl halides, glycosidic derivatives linkages, 6-aminocaproic acid. The linker may be is selected from the group consisting of: N-hydroxysuccinimide, N-oxysuccinimide, and N-hydroxysuccinimide diester (SIDEA).

When the reaction with the carrier protein and polysaccharide involves different functional groups, it will be understood that a heterobifunctional linker will be used capable to selectively react with both the different functional groups. In this case, preferred heterobifunctional linkers are selected from at least one of: succinimidyl 3-(2-pyridyldithio)propionate (SPDP), succinimidyl 6-(3-[2-pyridyldithio]-propionamido)hexanoate (LC-SPDP), sulfosuccinimidyl 6-(3′-(2-pyridyldithio)propionamido)hexanoate (sulfo-LC-SPDP), 4-succinimidyloxycarbonyl-a-methyl-a-(2-pyridyldithio)toluene (SMPT), sulfosuccinimidyl-6-[α-methyl-α-(2-pyridyldithio)tolueamideo]hexanoate (sulfo-LC-SMPT), succinimidyl 4-(N-maleimidomethyl)cyclohexane-1-carboxylate (SMCC), sulfosuccinimidyl 4-(N-maleimidomethyl)cyclohexane-1-carboxylate (suflo-SMCC), m-maleimidobenzoyl-N-hydroxysuccinimide ester (MBS), m-maleimidobenzoyl-N-hydroxysulfosuccinimide ester (sulfo-MBS), N-succinimidyl (4-iodoacetyl)aminobenzoate (SIAB), sulfosuccinimidyl (4-iodoacetyl)aminobenzoate (sulfo-SIAB), succinimidyl 4-(N-maleimidophenyl)butyrate (SMPB), sulfosuccinimidyl 4-(N-maleimidophenyl)butyrate(sulfo-SMPB), N-γ-maleimidobutyryl-oxysuccinimide ester (GMBS), N-γ-maleimidobutyryl-oxysulfosuccinimide ester (sulfo-GMBS), succinimidyl-6-((((4-(iodoacetyl)amino)methyl)cyclohexane-1-carbonyl)amino)hexanoate (SIACX), succinimidyl 6[6-(((iodoacetyl)amino)hexanoyl)amino]hexanoate (SIAXX), succinimidyl-4-(((iodoacetyl)amino)methyl)cyclohexane-1-carboxylate (SIAC), and succinimidyl 6-[(iodoacetyl)amino]hexanoate (SIAX) and p-nitrophenyl iodoacetate (NPIA).

The linker will generally be added in molar excess to polysaccharide during coupling to the polysaccharide. Conjugates may have excess carrier (w/w) or excess polysaccharide (w/w), e.g., in the ratio range of 1:5 to 5:1. Conjugates with excess carrier protein are typical, e.g., in the range 0.2:1 to 0.9:1, or equal weights. The conjugate may include small amounts of free (i.e., unconjugated) carrier. When a given carrier protein is present in both free and conjugated form in a composition of the invention, the unconjugated form is preferably no more than 5% of the total amount of the carrier protein in the composition as a whole, and more preferably present at less than 2% (by weight).

The composition may also comprise free carrier protein as immunogen (WO96/40242).

After conjugation, free and conjugated polysaccharides can be separated. There are many suitable methods, e.g., hydrophobic chromatography, tangential ultrafiltration, diafiltration, etc. (see also Lei et al. (2000) Dev Biol (Basel) 103:259-264 and WO00/38711). Tangential flow ultrafiltration is preferred.

The protein-polysaccharide conjugate is preferably soluble in water and/or in a physiological buffer.

For some polysaccharides, the immunogenicity may be improved if there is a spacer between the polysaccharide and the carrier protein. In this context, a ‘spacer’ is a moiety that is longer than a single covalent bond. This spacer may be a linker, as described above. Alternatively, it may be a moiety covalently bonded between the polysaccharide and a linker. Typically, the moiety will be covalently bonded to the polysaccharide prior to coupling to the linker or carrier. For example, the spacer may be moiety Y, wherein Y comprises a straight chain alkyl with 1 to 10 carbon atoms (e.g., C1, C2, C3, C4, C5, Ce, C7, C₈, C9, C10), typically 1 to 6 carbon atoms (e.g., C1, C2, C3, C4, C5, C6). The inventors have found that a straight chain alkyl with 6 carbon atoms (i.e., —(CH₂)₆) is particularly suitable, and may provide greater immunogenicity than shorter chains (e.g., —(CH₂)₂). Typically, Y is attached to the anomeric carbon of the polysaccharide, usually via an —O— linkage. However, Y may be linked to other parts of the polysaccharide and/or via other linkages. The other end of Y is bonded to the linker by any suitable linkage. Typically, Y terminates with an amine group to facilitate linkage to a bifunctional linker as described above. In these embodiments, Y is therefore bonded to the linker by an —N′H— linkage.

It will be appreciated that the one or more polysaccharide may have a variety of molecular weights, however, alternatively or additionally, the one or more polysaccharide has a molecular weight, or average molecular weight, of less than 100 kDa (e.g. less than 80, 70, 60, 50, 40, 30, 25, 20, 15, 14, 13, 12, 11, 10, 9, 8, 7, 6, 5, 4, 3, 2 or 1 kDa). In one embodiment at least one species of the one or more polysaccharides of has a molecular weight, or average molecular weight, of 7 kDa. By ‘average molecular weight’ we mean or include that the average (mean) molecular weight all of the polysaccharides of a given molecular species conjugated to the carrier polypeptide corresponds to the given value.

Likewise, the one or more polysaccharide may be of a variety of molecular weights, for example, alternatively or additionally, the one or more polysaccharide has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or fewer monosaccharide units. By ‘X or fewer monosaccharide units’ (wherein X represents a number between 1 and 30) we mean or include that the average (mean) number of monosaccharide units of one or more specified polysaccharide conjugated to the or each carrier polypeptide is X.

As mentioned, the one or more polysaccharide may be a bacterial polysaccharide such as a lipopolysaccharide (LPS) or capsular polysaccharide (CPS). Alternatively or additionally, where the one or more polysaccharide comprises or consists of a capsular polysaccharide of a bacterium it is selected from the group consisting of: Haemophilus influenzae type B and type A; Neisseria meningitidis serogroups A, C, W135, X and Y; Streptococcus pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F; Salmonella including Salmonella enterica serovar Typhi Vi, either full length or fragmented (indicated as fVi); Shigella sp, group A and B Streptococcus (GAS and GBS respectively). Preferably, the one or more polysaccharide is group A carbohydrate (GAC).

Alternatively or additionally, the polysaccharide may be conjugated to the carrier protein by any suitable means known in the art.

Alternatively or additionally, the one or more polysaccharide is conjugated to the carrier protein (a) by an amine formed from the reducing end residue from an aldehyde or ketone group from the terminal residue of the polysaccharide chain of the polysaccharide chain, and a lysine of the carrier protein; and/or (b) by one or more aldehyde groups formed from oxidised backbone and/or side chains of the polysaccharide (for example, for GAC, vicinal diols (1,2-diols) of the GlcNAc side chain) and a lysine of the carrier protein.

By ‘the reducing residue’ we mean or include aldehyde groups or ketone groups, particularly of the terminal sugar of polysaccharide chains (e.g., terminal 3-Deoxy-D-manno-oct-2-ulosonic acids [KDO] of O-antigen chains)

Alternatively or additionally, the polysaccharide conjugate further comprises an adjuvant, for example, aluminum hydroxide, Alhydrogel (aluminum hydroxide 2% wet gel suspension, Croda International Plc), and Alum-TLR7.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are disclosed for instance in Watson, Pediatr. Infect. Dis. J. (2000) 19:331-332. The use of an aluminium hydroxide and/or aluminium phosphate adjuvant is particularly preferred. These salts include oxyhydroxides and hydroxyphosphates. The salts can take any suitable form (e.g. gel, crystalline, amorphous, etc.).

Alternatively or additionally, the polysaccharide conjugate comprises or consists of:
I. the carrier polypeptide comprises or consists of the amino acid sequence according to SEQ ID NO: 1; and
II. the one or more polysaccharide conjugated to a carrier polypeptide comprises or consists of GAC (group A carbohydrate of Streptococcus pyogenes).

Alternatively or additionally, the polysaccharide conjugate comprises or consists of:
I. the carrier polypeptide comprises or consists of the amino acid sequence according to SEQ ID NO: 3; and
II. the one or more polysaccharide conjugated to a carrier polypeptide comprises or consists of GAC (group A carbohydrate of Streptococcus pyogenes).

Alternatively or additionally, the polysaccharide conjugate comprises or consists of:
I. the carrier polypeptide comprises or consists of the amino acid sequence according to SEQ ID NO: 5; and
II. the one or more polysaccharide conjugated to a carrier polypeptide comprises or consists of GAC (group A carbohydrate of Streptococcus pyogenes).

Alternatively or additionally, the polysaccharide conjugate comprises or consists of:
I. the carrier polypeptide comprises or consists of the amino acid sequence according to SEQ ID NO: 7 (CRM197); and
II. the one or more polysaccharide conjugated to a carrier polypeptide comprises or consists of GAC (group A carbohydrate of Streptococcus pyogenes).

Alternatively or additionally, the GAC:CRM₁₉₇ ratio may be 0.1:1, 0.2:1, 0.5:1, 0.7:1 0.9:1, 1:11:0.9, 1:0.7, 1:0.5, 1:0.2 or 1:0.1.

Polysaccharide conjugates of the invention are useful as active ingredients (immunogens) in immunogenic compositions, and such compositions may be useful as vaccines. Vaccines according to the invention may be prophylactic (i.e. to prevent infection) and/or therapeutic (i.e. to treat infection).

Alternatively or additionally, the carrier polypeptide of the polysaccharide conjugate of the invention is not CRM₁₉₇ or a variant, fragment or fusion thereof. Alternatively or additionally, the polysaccharide conjugate induces and/or is capable of inducing at least the same magnitude of anti-polysaccharide immune response as an otherwise equivalent polysaccharide conjugate having CRM₁₉₇ as the carrier polypeptide. The magnitude of the anti-polysaccharide immune response can be measured by any suitable means known in the art, but in one embodiment, is measured using ELISA (e.g., as described in the Examples section below and, in particular, the materials and methods therein). Alternatively or additionally, the polysaccharide conjugate induces and/or is capable of inducing an anti-polysaccharide immune response of at least 50% of the magnitude of an otherwise equivalent polysaccharide conjugate having CRM₁₉₇ as the carrier polypeptide, for example, at least 60%, 70%, 80%, 90%, 95%, 96%, 97%, 98%, 99% or at least 100%. Alternatively or additionally, the polysaccharide conjugate induces and/or is capable of inducing protective immunity of at least the same magnitude as an otherwise equivalent polysaccharide conjugate having CRM₁₉₇ as the carrier polypeptide, for example, at least 100%, 90%, 80%, 70%, 60%, 50%, 40%, 30% or at least 20%. Protective immunity can be determined using any suitable means in the art, for example, a mouse model (e.g., as described in the Examples section below and, in particular, the materials and methods therein, e.g., sections 4.6 and 4.7). Alternatively or additionally, the polysaccharide conjugate induces and/or is capable of inducing an anti-carrier polypeptide immune response of at least 50% of the magnitude as an otherwise equivalent polypeptide that has not been conjugated with polysaccharide, for example, at least 60%, 70%, 80%, 90%, 95%, 96%, 97%, 98%, 99% or at least 100%. The magnitude of the anti-carrier polypeptide immune response can be measured by any suitable means known in the art, but in one embodiment, is measured using ELISA (e.g., as described in the Examples section below and, in particular, the materials and methods therein). Alternatively or additionally, the polysaccharide conjugate induces and/or is capable of inducing protective immunity of greater or the same magnitude as an otherwise polypeptide that has not been conjugated with polysaccharide, for example, at least 200%, 175%, 150%, 140%, 130%, 120%, 110%, 100%, 90%, 80%, 70%, 60%, 50%, 40%, 30% or at least 20%. Protective immunity can be determined using any suitable means in the art, for example, a mouse model (e.g., as described in the Examples section below and, in particular, the materials and methods therein, e.g., sections 4.6 and 4.7).

Accordingly, a second aspect of the invention provides a vaccine comprising the polysaccharide conjugate of the first aspect.

Immunogenic compositions will be pharmaceutically acceptable. They will usually include components in addition to the antigens e.g. they typically include one or more pharmaceutical carrier(s), excipient(s) and/or adjuvant(s). A thorough discussion of carriers and excipients is available in Current Protocols in Molecular Biology (F. M. Ausubel et al., eds., 1987) Supplement 30, which is incorporated by reference herein. Thorough discussions of vaccine adjuvants are available in Vaccine Design: The Subunit and Adjuvant Approach (eds. Powell & Newman) Plenum Press 1995 (ISBN 0-306-44867-X); and Vaccine Adjuvants: Preparation Methods and Research Protocols (Volume 42 of Methods in Molecular Medicine series), ISBN: 1-59259-083-7. Ed. O'Hagan which are incorporated by reference herein.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilized during manufacture and are reconstituted into an aqueous form at the time of use. Thus, a composition of the invention may be dried, such as a lyophilized formulation. The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred. To improve thermal stability, a composition may include a temperature protective agent.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g., 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunizations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants.

Alternatively or additionally, the vaccine comprises an adjuvant (e.g., an adjuvant described in respect of the first aspect).

Alternatively, the vaccine comprises one or more additional polypeptide and/or polysaccharide antigen, for example, a bacterial antigen selected from the group consisting of antigens of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

Alternatively or additionally, the vaccine comprises unconjugated carrier protein. The unconjugated carrier protein may be present at less than or equal to 50% w/w as the conjugated carrier protein, for example, less than or equal to 40%, 30%, 20%, 10%, 5%, 1%, 0.1%, 0.01% or less than or equal to 0.01%. Alternatively or additionally, the vaccine comprises an immunologically effective amount of unconjugated carrier protein.

Accordingly, a third aspect of the invention provides a polysaccharide conjugate of the first aspect or a vaccine of the second aspect for use in medicine.

A fourth aspect of the invention provides a polysaccharide conjugate of the first aspect or a vaccine of the second aspect for use in raising an immune response in a mammal, for example, for treating and/or preventing one or more disease.

A fifth aspect of the invention provides a polysaccharide conjugate of the first aspect or a vaccine of the second aspect for raising an immune response in a mammal, for example, for treating and/or preventing one or more disease.

A sixth aspect of the invention provides a polysaccharide conjugate of the first aspect or a vaccine of the second aspect for the manufacture of a medicament for raising an immune response in a mammal, for example, for treating and/or preventing one or more disease.

A seventh aspect of the invention provides a method of raising an immune response in a mammal, the method comprising or consisting of administering the mammal with an effective amount of a polysaccharide conjugate of the first aspect or a vaccine of the second aspect.

Alternatively or additionally, the disease treated or prevented in the third to seventh aspects of the invention is an infection and/or symptom thereof of one or more bacterium selected from the group consisting of Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis). In particular, the disease treated or prevented in the third to seventh aspects of the invention is an infection and/or symptom thereof of Streptococcus pyogenes (i.e., Group A Streptococcus).

An eighth aspect of the invention provides a method of oxidising polysaccharide comprising the steps of:
I. oxidisation of polysaccharide by reacting:
   i. polysaccharide, for example, at a concentration of 0.1-100 mg/ml, e.g., 0.5-50, 0.5-25, 1-10, 2.5-7.5, 4-6 or 5 mg/mL, with
   ii. oxidising agent (for example, NaIO₄ [sodium periodate+, KMnO₄ [potassium permanganate], periodic acid [HIO₄], or lead tetra-acetate [Pb(OAc)₄]), at a concentration 0.5-10M,
   iii. in a suitable buffer (for example, phosphate buffer, or borate buffer) pH 3-9, for example, pH 5-8 (for example, pH5 or pH 8),
   iv. at a suitable temperature (for example, 20-30° C., such as 25° C.),
   v. for a suitable time (for example, 15 min-5 hr, such as, 30 min-3 hr, 30 min-1 hr, or 30 mins);
II. (optionally) quenching of residual NaIO₄ by:
   vi. addition of a suitable amount of reducing agent, for example, Na₂SO₃ (sodium sulfite), for example, at a molar excess with respect to the concentration of NaIO₄ in step I(ii), for example, 5-10 times the concentration of NaIO₄ in step I(ii), or 16 mM,
   vii. at a suitable temperature (e.g., 20-30° C., room temperature, or 25° C.),
   viii. for a suitable time (e.g., 10-30 min, or 15 min);
III. (optionally) purification and/or concentration of oxidised polysaccharide, for example, using a method selected from the group consisting of lyophilisation, centrifugal evaporation, rotary evaporation, and tangential flow filtration.

A ninth aspect of the invention provides a method of conjugating oxidised polysaccharide comprising the steps of:
A. reacting:
   a. oxidised polysaccharide (e.g., oxidised polysaccharide of the eighth aspect) at a concentration of 5-75 mg/mL (for example, 10-60 mg/mL, 20-50 mg/mL or 40 mg/mL) with;
   b. protein at a concentration of 5-75 mg/mL (for example 40 mg/mL); and
   c. NaBH₃CN (sodium cyanoborohydride) concentration of 0.5-10.0 mg/ml;
   d. In borate buffer or phosphate buffer pH 7-9, for example, pH 7.5-8.5, pH8;
   e. at a suitable temperature (for example, 17.5-42.5° C., room temperature, 25° C., 30° C. or 37° C.),
   f. for a suitable time (e.g., 1 hr, 2 hr, 4 hr, 6 hr, 0.5 to 3 days, 1 day or 2 days;
B. (optionally) quenching of residual aldehydes of oxidised polysaccharide by:
   a. addition of a suitable amount of NaBH₄ (e.g., an NaBH₄:polysaccharide ratio [w/w] of 0.5:1, or, for example, at a molar excess with respect to the aldehyde groups generated or moles of oxidized polysaccharide, for example, 5-10 times, 50 times, 100 times or 1000 times),
   b. at a suitable temperature (e.g., 20-30° C., 25° C., or room temperature),
   c. for a suitable time (e.g., 1 to 12 hr, 2-4 hr, 3 hr or 2 hr).
C. (optionally) purification of the polysaccharide conjugate resulting from step (B) by tangential flow filtration (TFF) and/or sterile filtration (e.g., TFF followed by sterile filtration).

Alternatively or additionally, conjugation yield is at least 5% higher than for traditional terminal reductive amination methods (i.e., the methods of Kabanova et al. [12] and described in section 4.2 of the present materials and methods section), for example, at least 10% 15%, 20%, 25%, 30%, 35%, 40%, 45%, 50%, 60%, 70%, 80%, 90%, 100%, 150% or 200% higher than traditional terminal reductive amination methods. Yield can be calculated by any suitable means known in the art but is preferably calculated using the methods described in the Examples section herein.

Alternatively or additionally, any of the methods above are configured to achieve at least 5%, at least 10%, at least 15%, between 10% and 30%, between 10% and 25%, or around 15% oxidation of the polysaccharide.

Alternatively or additionally, at least one of the polysaccharide concentration, the oxidising agent, the oxidising agent concentration, the suitable buffer, the suitable temperature and the suitable time used in any of the methods above may ensure that the method achieves at least 5%, at least 10%, at least 15%, between 10% and 30%, between 10% and 25%, or around 15% oxidation of the polysaccharide. Methods to determine whether an oxidation level has been reached, and suitable conditions to achieve different oxidation levels, are described in the Examples.

Alternatively or additionally, when the polysaccharide is GAC, any of the methods above can be configured to achieve a desired amount of GAC recovery.

In the context of oxidation, GAC recovery refers to the amount of oxidised GAC which is recovered after the GAC undergoes the oxidation process. Thus, GAC recovery as a percentage can be shown as the final amount of GAC (that is oxidized), divided by the starting amount of GAC, multiplied by 100.

Any of the oxidation methods above can be configured to achieve a GAC recovery of at least 60%, at least 65%, at least 70%, at least 75%, between 60% and 100%, between 65% and 100%, between 70% and 90%, or between 75% and 90%. At least one of the polysaccharide concentration, the oxidising agent, the oxidising agent concentration, the suitable buffer, the suitable temperature and the suitable time used in the method may ensure that the method achieves a GAC recovery of at least 60%, at least 65%, at least 70%, at least 75%, between 60% and 100%, between 65% and 100%, between 70% and 90%, or between 75% and 90%.

In the context of conjugation, GAC recovery refers to the amount of conjugated GAC that is recovered after the GAC undergoes the conjugation process. Thus, GAC recovery as a percentage can be shown as the final amount of conjugated GAC, divided by the starting amount of (oxidized) GAC, multiplied by 100.

Any of the conjugation methods above can be configured to achieve a GAC recovery of at least 25%, at least 30%, at least 35%, between 25% and 80%, between 30% and 70%, or between 35% and 60%. At least one of the oxidised polysaccharide concentration, the carrier polypeptide/protein concentration, the sodium cyanoborohydride concentration, the pH of the borate buffer, and the suitable temperature used in the method may ensure that the method achieves a GAC recovery of at least 25%, at least 30%, at least 35%, between 25% and 80%, between 30% and 70%, or between 35% and 60%.

Methods to determine whether a certain GAC recovery percentage has been achieved, and suitable ways to achieve certain GAC recovery percentages, are described in the Examples.

A tenth aspect of the invention provides a method of conjugating polysaccharide to polypeptide comprising the methods of the eighth and ninth aspects of the invention. Alternatively or additionally, the polysaccharide is a polysaccharide described in the first aspect of the invention, for example, GAC.

Alternatively or additionally, the protein is a protein described in the first aspect, for example, SpyAD (e.g., SEQ ID NO: 1 or SEQ ID NO: 2), SpyCEP (e.g., SEQ ID NO: 3 or SEQ ID NO: 4), Slo (e.g., SEQ ID NO: 5 or SEQ ID NO: 6) or CRM197 (e.g., SEQ ID NO: 7). Alternatively or additionally, the method product is a polysaccharide conjugate described in the first aspect of the invention, for example:
I. SpyAD (e.g., SEQ ID NO: 1 or SEQ ID NO: 2) conjugated to GAC;
II. SpyCEP (e.g., SEQ ID NO: 3 or SEQ ID NO: 4) conjugated to GAC;
III. Slo (e.g., SEQ ID NO: 5 or SEQ ID NO: 6) conjugated to GAC; or
IV. CRM₁₉₇ (e.g., SEQ ID NO: 7) conjugated to GAC.

Alternatively or additionally, reactions are performed below the Tm of the polypeptide, for example, at least 0.5, 1.0, 1.5, 2.0, 2.5, 3.0, 4.0, 5.0, 6.0, 7.0 or 7.5° C. below the Tm of the polypeptide.

An eleventh aspect of the invention provides a polysaccharide conjugate produced according to the method of the tenth aspect of the invention.

Preferred, non-limiting examples which embody certain aspects of the invention will now be described, with reference to the following figures.

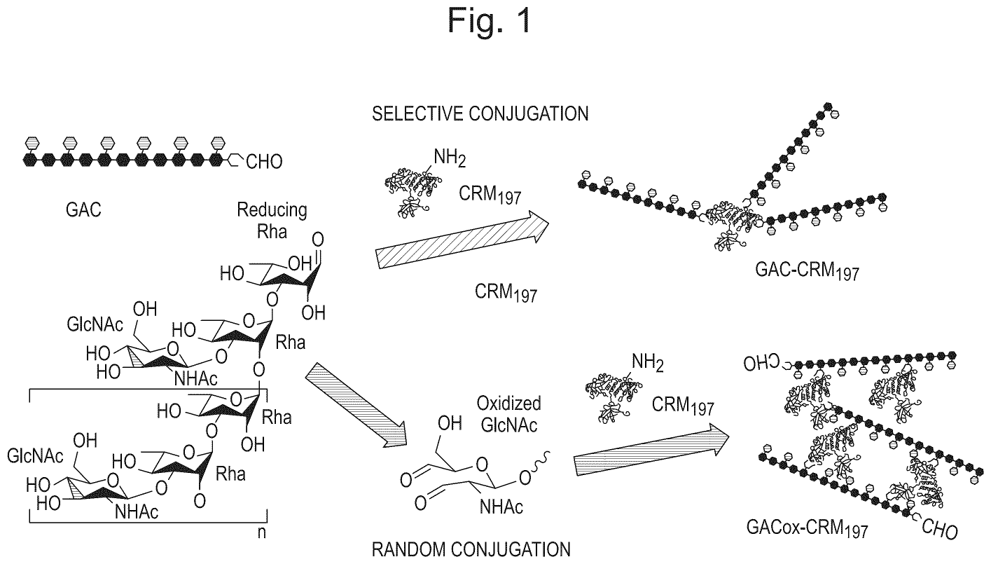

FIG. 1. Conjugation strategies for producing GAC conjugates: selective direct reductive amination between the aldehyde group at the reducing residue of GAC and lysines of the carrier protein (“selective conjugation” approach) [12] and reductive amination between the aldehyde groups randomly generated through oxidization of GAC and lysines of the carrier protein (“Random conjugation” approach).

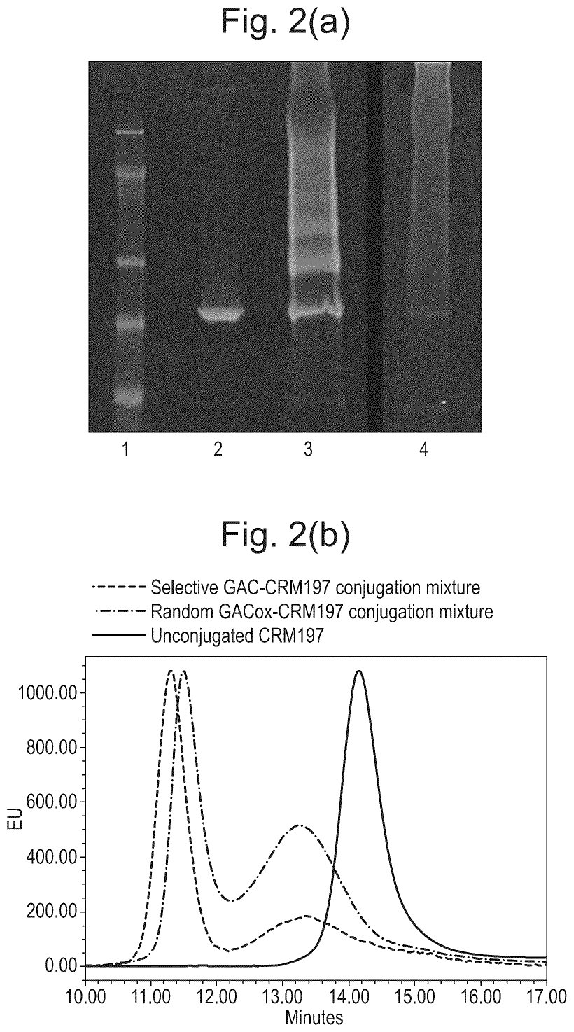

FIG. 2. (a) Characterization by SDS-PAGE analysis (7% Tris-acetate gel) of the conjugation mixtures in comparison to unconjugated CRM197. Ten μg of conjugated protein and 2 μg of unconjugated CRM197 were loaded per well. Lane 1: marker, lane 2: CRM197, lane 3: selective GAC-CRM197, lane 4: random GACox-CRM197. (b) HPLC-SEC profiles (fluorescence emission detection) of selective GAC-CRM197 conjugation mixture, random GACox-CRM197 conjugation mixture and unconjugated CRM197, 80 μL of sample injected on a TSK gel G3000 PWXL column; 0.1 M NaCl 0.1 M NaH2PO4 5% CH3CN pH 7.2 at 0.5 mL/min. Vtot 23.326 min, VO 10.663 min.

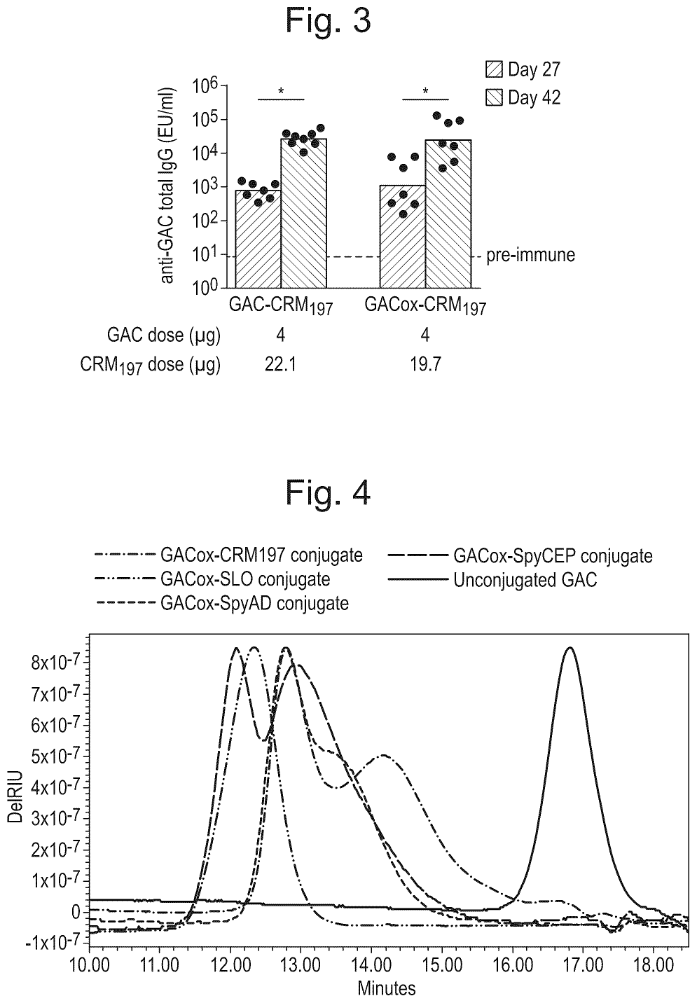

FIG. 3. Immunogenicity of GAC when conjugated to CRM197 through different chemistries. CD1 mice were immunized i.p. at day 0 and 28 with 4 μg/GAC dose formulated with 2 mg/mL Alhydrogel. Summary graph of anti-GAC specific IgG geometric mean units (bars) and individual antibody levels (dots) is reported (GAC-HSA used as coating antigen). Mann-Whitney two-tailed test was performed to compare the response induced by the two immunization groups (p>0.05) whereas Wilcoxon test was performed to compare the responses for each group at day 27 and day 42 (* P<0.05).

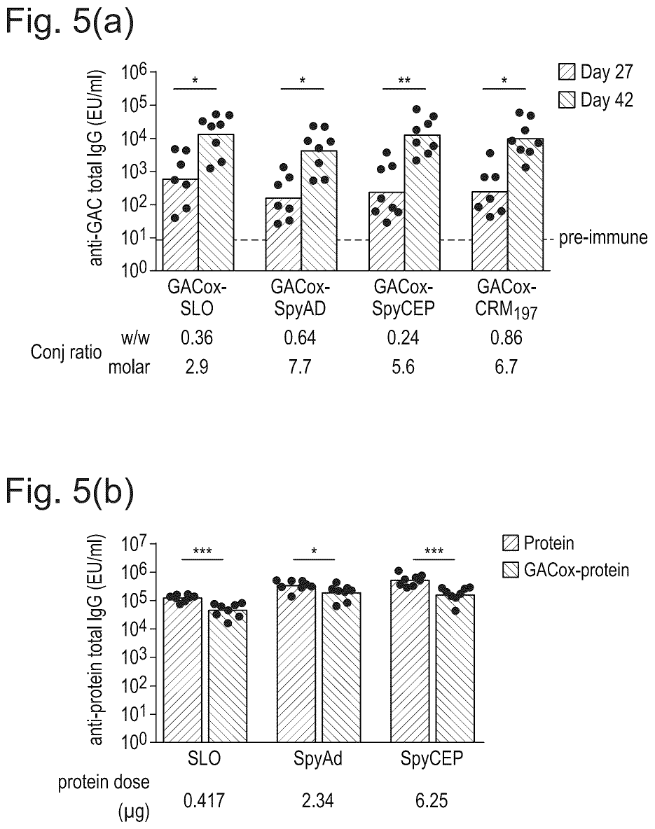

FIG. 4. HPLC-SEC profiles (refractive index detection) of GACox-CRM197, GACox-SLO, GACox-SpyAD, GACox-SpyCEP conjugates compared to unconjugated GAC. 80 μL of sample injected on a TSK gel G3000 PWXL column; 0.1 M NaCl 0.1 M NaH2PO4 5% CH3CN pH 7.2 at 0.5 mL/min. Vtot 23.326 min, VO 10.663 min.

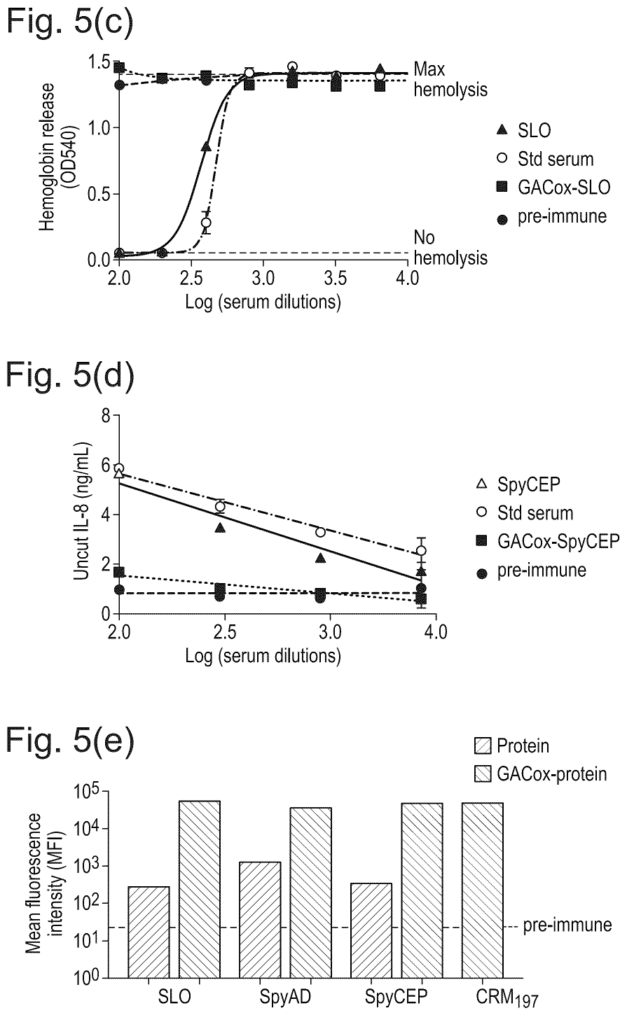

FIG. 5. Immunogenicity of GAC when conjugated to CRM197 or GAS proteins SLO, SpyAD and SpyCEP. CD1 mice were immunized i.p. at day 0 and 28 with 1.5 μg/GAC dose or with the corresponding dose of the carrier protein alone, all formulated with 2 mg/mL Alhydrogel. Sera were analysed by ELISA using as coating antigens GAC-HSA (a) or SLO, SpyAD and SpyCEP (b). Summary graphs of anti-antigen specific IgG geometric mean units (bars) and individual antibody levels (dots) are reported. Kruskal-Wallis test was performed among the 4 groups in graph (a), Wilcoxon test was performed between response at day 27 and day 42 in graph (a) (p>0.05) and Mann-Whitney two-tailed test between each group immunized with protein alone or GACox-protein conjugate in graph (b) (* P<0.05, ** P<0.01, *** P<0.001). Sera were tested in the hemolysis inhibition assay (c) and in the IL-8 cleavage inhibition assay (d) to evaluate their ability to block native SLO and SpyCEP activity, respectively. The amount of hemoglobin released by rabbit red blood cells (c) and of uncut IL-8 (d) observed at each serum dilution tested is reported for pre-immune serum, standard serum and one selected day 42 serum for each immunization group. Pooled sera at day 42 were tested in FACS (e) to evaluate their ability to bind to GAS bacterial cells. Following incubation of bacteria with the different sera, APC-conjugated anti-mouse IgG secondary antibody was used for detection. The mean fluorescence intensity (MFI) measured for each serum is reported as compared to pre-immune sera.

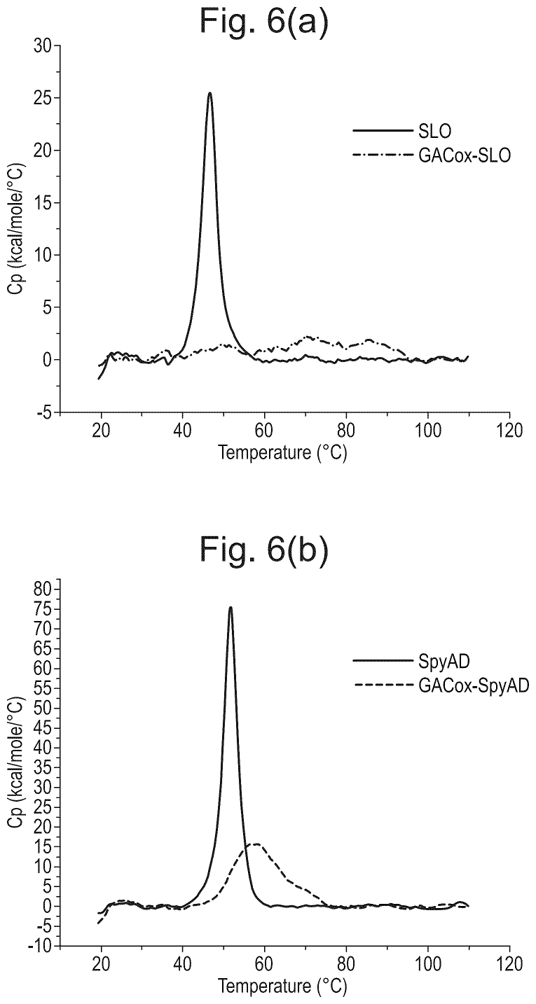

FIG. 6. DSC thermograms of (a) unconjugated SLO vs GACox-SLO and (b) unconjugated SpyAD vs GACox-SpyAD. GAS proteins and corresponding conjugates were analyzed in phosphate buffer at pH 7.2, at the same molar concentration of 3 μM for SLO and 2 μM for SpyAD. The AH values (from the integrated areas under the curves) for each thermogram were: SLO: 1.3E5 kcal/mole; GACox-SLO: nd; SpyAD: 3.7E5 kcal/mole; GACox-SpyAD: 2.4E5 kcal/mole.

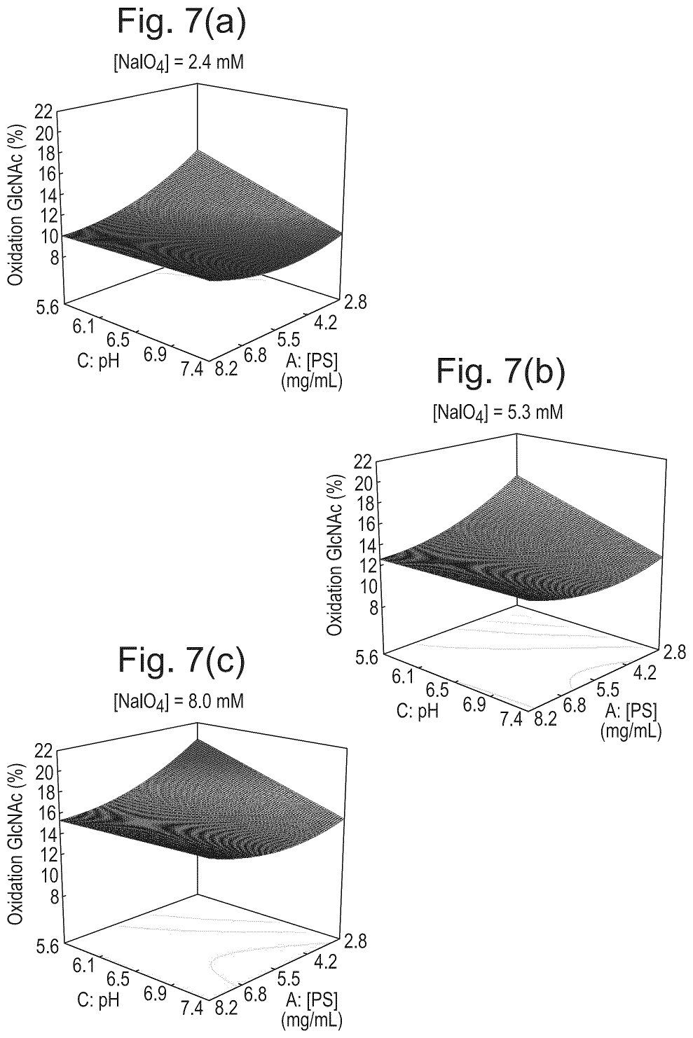

FIG. 7. Identification of optimal conditions for GAC oxidation: 3D Surface Model Graphs for % GlcNAc oxidation response. Correlation between pH and GAC concentration at NaIO4 concentration of 2.4 (a), 5.3 (b), 8.0 (c).

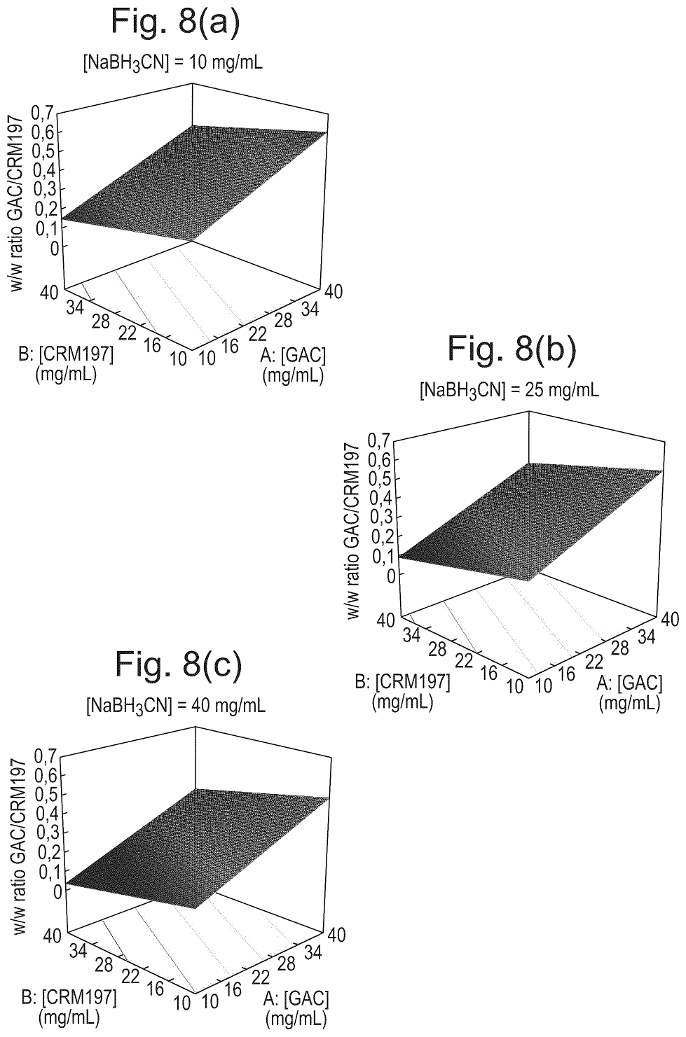

FIG. 8. Identification of optimal conditions for GACox conjugation to CRM197: 3D Surface Model Graphs for GAC/CRM197 w/w ratio (a-c) and GAC yield (d-f) responses. Correlation between CRM197 and GAC concentrations at NaBH3CN concentrations of 10 (a,d), 25 (b,e) and 40 (c,f).

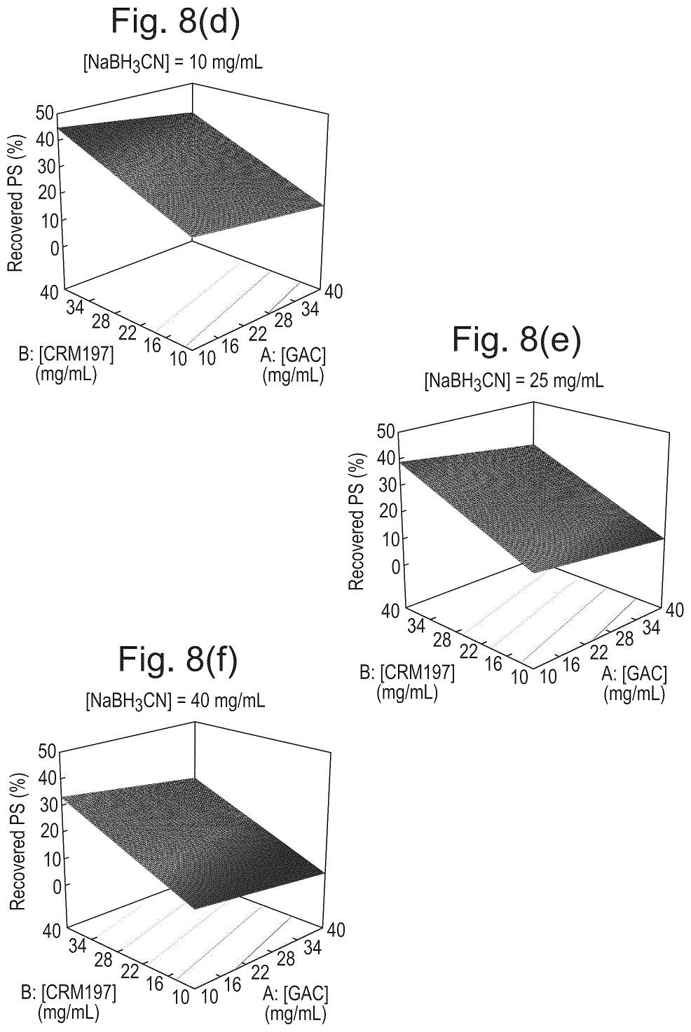

FIG. 9. Scheme 1. Flow chart of GAC to CRM197 optimized conjugation process.

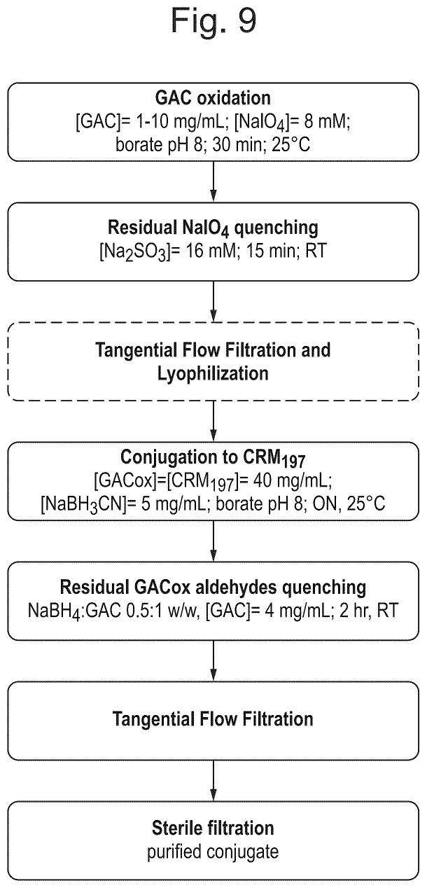

FIG. 10 (FIG. S1). Immunogenicity of GAC when conjugated to CRM197 through different chemistries. CD1 mice were immunized i.p. at day 0 and 28 with 4 μg GAC/dose formulated with 2 mg/mL Alhydrogel. Summary graph of anti-CRM197 specific IgG geometric mean units (bars) and individual antibody levels (dots) is reported (CRM197 used as coating antigen). Mann-Whitney two-tailed test was performed to compare the response induced by the two immunization groups (p>0.05).

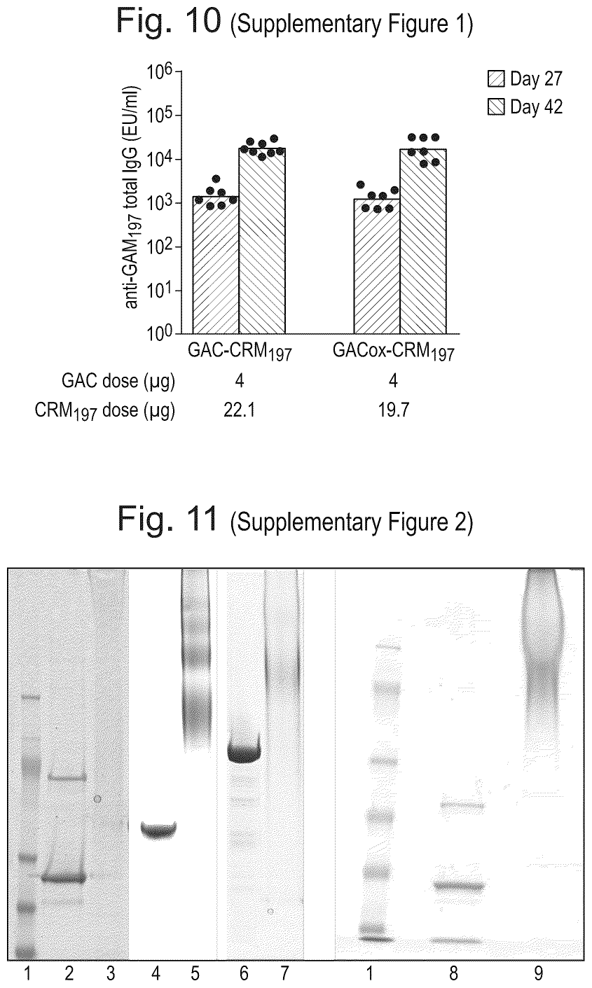

FIG. 11 (FIG. S2). Characterization by SDS-PAGE analysis (3-8% Tris-acetate gel for GAS proteins conjugates, 7% Tris-acetate gel for CRM197 conjugate) of the conjugation mixtures in comparison to corresponding unconjugated proteins. Ten μg of conjugates and 2 μg of unconjugated proteins were loaded per well. Lane 1: marker, lane 2: SLO; lane 3: SLO conjugate; lane 4: SpyAD; lane 5: SpyAD conjugate; lane 6: SpyCEP; lane 7: SpyCEP conjugate; lane 8: CRM197, lane 9: CRM197 conjugate.

### EXAMPLES

### Introduction

No commercial vaccine is yet available against Group A Streptococcus (GAS), major cause of pharyngitis and impetigo, with a high frequency of serious sequelae in low- and middle-income countries. Group A Carbohydrate (GAC), conjugated to an appropriate carrier protein, has been proposed as an attractive vaccine candidate. Here, we explored the possibility to use GAS Streptolysin O (SLO), SpyCEP and SpyAD protein antigens with dual role of antigen and carrier, to enhance the efficacy of the final vaccine and reduce its complexity. All protein antigens resulted good carrier for GAC, inducing similar anti-GAC IgG response to the more traditional CRM197 conjugate in mice. However, conjugation to the polysaccharide had a negative impact on the anti-protein responses, especially in terms of functionality as evaluated by an IL-8 cleavage assay for SpyCEP, and a hemolysis assay for SLO. After selecting CRM197 as carrier, optimal conditions for its conjugation to GAC were identified through a Design of Experiment approach, improving process robustness and yield This work supports the development of a vaccine against GAS and shows how novel statistical tools and recent advancements in the field of conjugation can lead to improved design of glycoconjugate vaccines.

### 2. Results

2.1. Testing Random and Selective Conjugation Chemistries for Linkage of GAC to CRM₁₉₇

Two different approaches were compared for conjugation of GAC to CRM₁₉₇, one of the most extensively and successfully carrier proteins used in glycoconjugate vaccines [21]. The selective direct reductive amination between the aldehyde group at the reducing residue of GAC and lysines of the carrier protein [12] resulted in a conjugate characterized by GAC to CRM₁₉₇ w/w ratio of 0.18, corresponding to an average of 1.5 chains of GAC per molecule of the carrier. When we produced additional conjugate lots by using the same conjugation conditions, there was large batch-to-batch inconsistency with GAC to CRM₁₉₇ ratios ranging between 0.01 and 0.18. Moreover, in some occasions, no conjugate formation was verified.

An alternative random approach was tested, that still relies on a reductive amination chemistry. In particular, a step of random GAC oxidation with sodium periodate was introduced, producing additional aldehydic groups along the polysaccharide chains. The oxidation occurs at the vicinal diols of the GlcNAc side chain of GAC. The reductive amination of oxidized GAC was performed with the same conditions used for linkage of GAC via its reducing end (GAC concentration of 10 mg/mL, GAC to CRM₁₉₇ to NaBH₃CN w/w/w ratio of 4:1:2, 200 mM phosphate buffer at pH 8, 2 days at 37° C.), resulting in a conjugate with GAC to CRM₁₉₇ w/w ratio of 0.2, similar to that of the selective conjugate. The two conjugation schemes are reported in FIG. 1.

As expected, random and selective conjugates showed a different protein pattern by sodium dodecyl sulfate-polyacrylamide gel electrophoresis (SDS-PAGE) analysis: single bands at increasing molecular weight (MW) for the selective approach, corresponding to increasing number of GAC chains linked to CRM₁₉₇ vs a polydisperse smear at very high MW for the random conjugate (FIG. 2(a)). Conjugate formation was also confirmed by High Performance Liquid Chromatography-Size Exclusion Chromatography (HPLC-SEC) (FIG. 2(b)). The profiles of the two conjugates differed significantly from SDS-PAGE patterns. In fact, differently from what expected, the random conjugate showed a main peak at slightly higher retention time compared to the selective conjugate. Indeed, HPLC-SEC estimates an apparent MW that can reflect the different structure of the two constructs. HPLC-SEC analysis also confirmed absence of free CRM₁₉₇ in both conjugation mixtures. Residual unconjugated GAC was removed by size exclusion chromatography on Sephacryl S-100 HR column. Total GAC recoveries after purification were approximately 5% for both conjugates.

The two conjugates produced via random and selective approaches were compared in mice, to check if random linkage of GAC to the protein could negatively impact on the induced immune response. Both conjugates induced no significant different anti-GAC IgG response 4 weeks after the first immunization, with similar booster (p<0.05) 2 weeks after the second dose (FIG. 3). Similar anti-CRM₁₉₇ IgG responses were also induced (FIG. S1).

2.2. Applying Random Chemistry for Linkage of GAC to GAS Proteins

To increase GAC recovery, the reductive amination step conditions were slightly modified (GAC concentration increased from 10 to 40 mg/mL, GAC to CRM₁₉₇ to NaBH₃CN w/w/w ratio of 4:1:2, borate buffer at pH 8 instead of phosphate [23], 2 days at 37° C.), resulting in a conjugate with GAC/CRM₁₉₇ w/w ratio increased from 0.2 to 0.86 and GAC yield from 5% to 21.5%.

The same conditions were applied for linking GAC to GAS SLO, SpyAD and SpyCEP protein antigens. However, because the melting temperature (Tm) by Differential Scanning Calorimetry (DSC) for these proteins resulted to be close to 37° C. (Tm of 39.35° C. for SLO, 44.37° C. for SpyAD, 40.03° C. for SpyCEP), the reactions were performed at 25° C. instead of 37° C., trying to preserve GAS proteins folding and, possibly, functionality in the final conjugates.

Conjugate formation was confirmed by SDS-PAGE for all the conjugates, also revealing absence of free proteins (FIG. S2). Purification by Amicon 30 kDa cut-off successfully reduced level of free GAC to <10% for all conjugates (FIG. 4(b)), as verified by HPLC-SEC (refractive index detection) (FIG. 4), also confirming conjugate formation. The conjugates were characterized by a similar GAC/protein molar ratio, higher than with SLO (Table 1).

TABLE 1
Main characteristics of purified GAC conjugates
with CRM₁₉₇ and GAS proteins.

| Conjugate | GAC/protein molar ratio | GAC/protein w/w ratio |
|---|---|---|
| GACox-CRM₁₉₇ | 7.2 | 0.86 |
| GACox-SLO | 3.3 | 0.36 |
| GACox-SpyAD | 8.2 | 0.64 |
| GACox-SpyCEP | 6.0 | 0.24 |

When compared in mice, the conjugates with the GAS protein antigens induced same anti-GAC IgG response compared to GAC-CRM₁₉₇ both 4 weeks after first and 2 weeks after second injection, showing that all GAS proteins tested were good carriers for GAC. All conjugates were able to elicit a booster response after re-injection (FIG. 5(a)). Importantly, the physical mixture of GAC with one of the carrier proteins tested did not give a significant anti-GAC IgG response, confirming the role of the carrier protein at inducing T-cell activation and isotype switching.

A Flow cytometry analysis (FACS) against GAS bacterial cells was performed with pooled sera collected 2 weeks after the second injection from each immunization group (FIG. 5(e)). Antibodies induced by all conjugates were able to similarly bind the bacterial cells. Sera induced by unconjugated GAS proteins bound GAS bacteria to a less extent compared to the corresponding conjugates.

However, when GAS proteins were used as carrier, anti-protein-specific total IgG decreased if compared with immunization with the same dose of unconjugated protein (FIG. 5(b)). The effect of conjugation of GAC to GAS proteins was evident when serum functionality was analyzed. Conjugation of GAC completely abolished the ability of SLO and SpyCEP to elicit antibodies able to block native SLO hemolytic activity (FIG. 5(c)) and native SpyCEP protease activity (FIG. 5(d)), respectively.

Through a DSC analysis, strong impact of conjugation on SLO and SpyAD folding was verified, probably correlated with the loss of functionality evidenced. For SLO, folding was not retained at all after conjugation, whereas for SpyAD a decrease in the enthalpy change ([1]) was observed (FIG. 6).

Thus, based on the results obtained, CRM₁₉₇ was selected as the best carrier for GAC and the conjugation process was further optimized through a DoE approach with the main aim to maximize GAC yield and assure robustness of the process.

2.3. Optimization of the Random Chemistry Through a DoE Approach

2.3.1. Identification of Optimal Conditions for GAC Oxidation

After performing some preliminary experiments, a first DoE was performed to understand which parameters could affect the GAC oxidation step, aiming at identifying their best combination to obtain optimal oxidation degree for efficient conjugation, preventing major impact on GAC structural integrity.

A full factorial, response surface design, with alpha of 1.68179 (rotatable), with 1 replicate of axial and factorial points and 6 center point replicates, was used.

GAC concentration in the range 1-10 mg/mL, pH in the range 5-8, and NaIO₄ concentration in the range 0.5-10 mM were the factors evaluated. Reaction time and temperature were set, respectively, at 30 minutes and 25° C. Conditions used for oxidation and results are summarized in Table S1.

Similar GAC recoveries were obtained in all reaction conditions. We also verified no impact on polysaccharide chain length, as expected as GlcNAc, that is the sugar impacted by the oxidation, is in the side chain and not in the backbone of GAC.

In the design space tested, the % GlcNAc oxidation was in the range 8.5-19.4%, meaning that a maximum of 3 repeating units as average per PS chain were oxidized (considering an average of 14 repeating units per GAC chain).

To elaborate the data, a response surface with a quadratic model was chosen and with a backward elimination process, the non-significant terms (p-value >0.05) were removed from the model (statistical analysis in Table S3). The residuals (externally studentized) were normally distributed (Anderson-Darling normality test, p=0.837) and the model resulted with an adjusted-R2 of 0.71.

The GlcNAc oxidation response was affected by all factors investigated, and mainly by NaIO₄ concentration (p=0.0003) (FIG. 7).

From the model we achieved a target of oxidation of 15%. Working at pH 8, allowed quenching of NaIO₄ excess with Na₂SO₃ and the subsequent conjugation without GACox intermediate purification. By fixing the pH at 8, the target oxidation level could be reached by working with 8 mM NaIO₄, quite independently from GAC concentration in the range investigated.

2.3.2. Identification of Optimal Conditions for GAC Conjugation to CRM₁₉₇

After having identified optimal conditions for GAC oxidation, the DoE approach was used to understand which parameters are critical for the conjugation step and to identify their optimal combination to maximize GAC yield, ensuring robustness of the process.

A full factorial, response surface design, with alpha of 1.0 (face centered), with 1 replicate of axial and factorial points and 6 center point replicates, was used.

GACox, CRM₁₉₇ and NaBH₃CN concentrations were the factors evaluated, all tested in the range 10-40 mg/mL. Reaction time, temperature and pH were set, respectively, at 2 days, 25° C. and pH 8 in borate buffer. Conditions used for the conjugation tests and results obtained are summarized in Table S2.

Unconjugated CRM₁₉₇ was >10% only in 3 of the 20 tests performed and absent in 15 of them, as calculated by HPLC-SEC analysis. In the design space tested, GAC/CRM₁₉₇ w/w ratios were in the range 0.12-0.65, whereas GAC recovery ranging from 9.2 to 41.9%, as calculated by Anion Exchange Chromatography coupled with Pulsed Amperometric Detection (HPAEC-PAD).

To elaborate the data, for either GAC/CRM₁₉₇ w/w ratios and GAC yields, a response surface with a linear model was chosen. The non-significant terms (p-value >0.05) were removed from the models using a backward elimination process (statistical analyses in Table S4). The residuals (externally studentized) for both models were normally distributed. Normality was calculated through the Anderson-Darling test (p=0.166 for GAC/CRM₁₉₇ w/w ratio and p=0.676 for GAC yield) and the models resulted with adjusted-R2 of 0.87 and 0.83 for GAC to protein ratio and GAC recovery, respectively.

For both the responses evaluated, all factors investigated in the DoE affected the responses (FIG. 8). Interestingly, GAC/CRM₁₉₇ w/w ratio and GAC recovery increased by reducing NaBH₃CN concentration.

Based on the results, optimization was done, with all factors in range, maximizing the % of GAC recovery with higher importance than for GAC/CRM₁₉₇ w/w ratio maximization.

Optimal conditions identified are reported in Table 2, along with predicted responses and with the actual results obtained by performing the conjugation in the identified reaction conditions. Results obtained were in agreement with those expected, confirming consistency of the process, as all the responses obtained were within the 95% of confidence interval (CI) for Mean.

TABLE 2
Optimized conditions for GACox-CRM conjugation
and predicted responses from the model, confirmed
by performing an additional conjugation test.

| | GAC/CRM₁₉₇ w/w | | GAC recovery % | |
|---|---|---|---|---|
| | Predicted | | Predicted | |
| | (95% CI | | (95% CI | |
| Optimized conditions | for Mean) | Actual | for Mean) | Actual |
| [GACox] = [CRM₁₉₇] = | 0.46 | 0.39 | 38 | 39 |
| 40 mg/mL; | (0.39-0.52) | | (32-43) | |
| [NaBH3CN] = 10 mg/mL; | | | | |
| borate buffer pH 8; | | | | |
| T = 25° C.; | | | | |
| 2 days reaction time | | | | |

Having identified NaBH₃CN concentration as a critical factor for the process, additional conjugation tests were performed further decreasing NaBH₃CN concentration from 10 to 5 and 1 mg/mL, to check if further lowering the concentration of this reagent could be beneficial to conjugation efficiency. Furthermore, role of reaction time was investigated, running the conjugations at 4 h, overnight (ON) or for 2 days. The other parameters were kept the same, as per DoE optimization. Carrying out the reaction with 5 and 1 mg/mL of reducing agent resulted in conjugates with slightly higher GAC to CRM₁₉₇ ratio compared to 10 mg/mL NaBH₃CN concentration (Table 3).

TABLE 3
Investigating role of NaBH₃CN concentration and
reaction time on GACox conjugation to CRM₁₉₇.

| | | |
|---|---|---|
| [NaBH₃CN] in | | GAC/CRM₁₉₇ w/w ratio |
| reaction (mg/mL) | Reaction time | in purified conjugate |

| 10 | 4 | h | 0.34 |
|---|---|---|---|
| | ON | | 0.41 |
| | 2 | days | 0.39 |
| 5 | 4 | h | 0.36 |
| | ON | | 0.46 |
| | 2 | days | 0.48 |
| 1 | 4 | h | 0.42 |

| ON | | 0.45 |
|---|---|---|
| 2 | days | 0.47 |

Further reducing NaBH₃CN concentration from 1 to 0.25 mg/mL negatively impacted on GAC to CRM₁₉₇ ratio and GAC recovery % (data not shown). Based on such results, 5 mg/mL NaBH₃CN was selected and reaction time reduced to ON. Optimized conjugation process is described in FIG. 9 (Scheme 1).

The process was also scaled up to 100 mg GAC, further confirming robustness of the process, as the resulting conjugate was characterized by similar GAC to CRM₁₉₇ w/w ratio and GAC % yield compared to a conjugate produced at 10 mg scale (Table 4) and again the results obtained were within the 95% CI for Mean from the DoE optimization reported in Table 2.

TABLE 4
Conjugates produced at different scale in optimized
conditions confirming expected results in terms of GAC
to CRM₁₉₇ ratio and process yield.

| | |
|---|---|
| GAC/CRM₁₉₇ w/w | GAC recovery % |

| Small scale | Large scale | Small scale | Large scale |
|---|---|---|---|
| 0.44 | 0.51 | 44 | 39 |

Such conjugate was tested in mice confirming its ability to induce an anti-GAC IgG response comparable to that elicited by the CRM₁₉₇ conjugate produced before the optimization (FIG. 5).

### 3. Discussion

No licensed vaccine is yet available against GAS, a leading cause of global morbidity and mortality worldwide, responsible for a wide range of diseases and estimated to cause about 0.5 million annual deaths, mostly in young adults [2]. One of the main barriers to vaccine development is related to the high GAS strain diversity, serologically based on the serotype of the surface M protein [15], one of the major virulence and immunological determinants of GAS [24]. To date only M protein based candidate vaccines have been tested in clinical trials [6, 25-27], but novel vaccines based on conserved protein antigens and surface polysaccharide are also in development [28]. The highly conserved SLO, SpyAD, SpyCEP, and GAC conjugated to a carrier protein have been proposed as an attractive alternative vaccine candidate [6].

Here, these three protein antigens have been tested as carrier for GAC, with the aim to simplify the final vaccine design, combining two of the four antigens in one construct. To date, only few carrier proteins have been used for licensed glycoconjugate vaccines and there is increased concern for carrier-induced epitope suppression (CIES), that could result in reduced anti-carbohydrate immune response after patient repeated exposure, simultaneously or in close sequence, to a given carrier [21, 29, 30]. The identification of new carriers is driven also by the interest to explore the dual role as carrier and antigen that a pathogen-related protein can play, thus resulting in a vaccine that, by simultaneous administration of carbohydrate and protein antigens, tackles two different virulence factors of the pathogen [21]. Such type of combinations has already been proposed and investigated at the preclinical level [31-36]. Among these, also few GAS proteins have been explored as possible carriers. A variant of GAC chain, conjugated to GAS arginine deiminase (ADI) protein antigen, was able to protect from superficial skin infection, but not against invasive GAS disease, in a challenge study in mice [37]. GAC oligosaccharides, conjugated to an inactive mutant of GAS C5a peptidase (ScpA), ScpA193, induced robust anti-carbohydrate immune responses in mice. Antibodies induced mediated GAS opsonophagocytosis in vitro, as well as effectively protected animals from GAS challenges and GAS-induced pulmonary damage. However, anti-ScpA193 antibodies induced by the protein alone had only moderate binding activity to GAS cells and no opsonophagocytic activity, despite the high titers induced [38, 39].

Either SLO, SpyAD and SpyCEP proteins tested here have proven to be good alternative carriers to the benchmark CRM₁₉₇ for promoting anti-GAC IgG response as well as binding to GAS bacteria by FACS (FIG. 5). These results make these proteins attractive as new carrier proteins, potentially to be used also with other PS antigens.

Anti-protein specific antibodies induced by the conjugates were maintained, albeit at levels lower than those induced by the proteins alone A bioconjugate vaccine produced with S. aureus type 5 capsular PS (CP5) linked to S. aureus α toxin (Hla) has been already shown to be protective against both bacteremia and lethal pneumonia, providing broad-spectrum efficacy against staphylococcal invasive disease, with specific protective antibodies induced against both the glycan and the protein moiety [44].

Here, a more traditional semi-synthetic approach was used for conjugation of GAC. The conjugation chemistry used, which actually affects the efficiency of conjugation, saccharide to protein ratio and glycoconjugate structure and size, is one of the parameters that can mostly impact the immunogenicity of glycoconjugate vaccines [45-47]. We compared terminal linkage of GAC to CRM₁₉₇ with a random approach. Both conjugates elicited similar immune response in mice. In principle, the use of selective chemistry, resulting in more homogeneous and well-defined structures with no impact on sugar chains, should be preferable in terms of production consistency. Nevertheless, in our case the use of the selective approach resulted in batch-to-batch inconsistency with no conjugate formation in some cases. Introduction of few more reactive aldehyde groups along the GAC chain, compared to the aldehyde group on the terminal reducing end of the sugar, allowed more reproducible conjugation. From a process perspective, the synthesis of the random conjugate requires one more step compared to the selective one. However, by quenching the excess of the oxidizing agent with sodium sulfite, the carrier protein could be directly added in the mixture avoiding GACox intermediate purification and simplifying the process to one step only (FIG. 9—Scheme 1).

As a random approach leads to the formation of cross-linked and rather undefined and heterogeneous structures, a careful and tight control of the manufacturing process is essential to guarantee consistency together with a proper analytical characterization [46].

Here, a DoE approach was used to identify optimal conjugation conditions for assuring process robustness and improving yields. Yield increase means reducing cost-of-goods to have a more sustainable and affordable product, an important matter to meet the vaccination demand in LMICs.

The DoE methodology, compared to the traditional one-factor-at-a-time (OFAT) approach, allows identification of optimal combination of the critical parameters, considering their interaction, and to model the process in the design space investigated, predicting impact of changes in the critical parameters of the quality of the final product [52, 53]. In the vaccines field, DoE has been used for development or optimization of analytical and immunological assays [54-56] or for improving vaccine formulations or purification processes [57-60].

In our study, DoE has been used for optimizing a conjugation process. Through this approach, conjugation yield has been increased from 5% to around 40% and process robustness has been assured and confirmed, also scaling up the process to 100 mg-scale of GAC.

In conclusion, this work supports the development of a universal vaccine against GAS and shows how novel tools can be used for the design of improved vaccines, with the final goal to ensure consistent delivery of safe and efficacious products with robust manufacturing processes.

### 4. Materials and Methods

4.1. Materials

GAC was extracted from a M protein-mutant strain (GAS51AM1) generated from the wild-type strain HRO-K-51 kindly provided by the University of Rostock. GAS recombinant proteins SpyAD (SpyADstop, 89.5 kDa, 62 lysines in total) and SpyCEP (SpyCEP double mutant, 174.0 kDa, 133 lysines in total) were produced and purified at GVGH as previously described [17, 61], GAS recombinant protein SLO (SLO double mutant, 60.6 kDa, 56 lysines in total) and CRM₁₉₇ (58.4 kDa, 39 lysines in total) were obtained from GSK R&D.

GAC was chemically extracted from bacterial culture through nitrite/glacial acetic acid treatment [51]. The purification was performed using a combination of tangential flow filtration and anionic exchange chromatography, as previously described [12]. Purified GAC contained no hyaluronic acid, <4% protein and <1% DNA impurities (w/w with respect to GAC). Average molecular size of 7.0 kDa was estimated by HPLC-SEC analysis (TSK gel G3000 PW_{XL} column) using dextrans (5, 25, 50, 80, 150 kDa) as standards (Merck), corresponding to an average of 14 repeating units per chain.

The following chemicals were used in this study: sodium phosphate monobasic (NaH₂PO₄), sodium phosphate dibasic (Na₂HPO₄), sodium cyanoborohydride (NaBH₃CN), sodium periodate (NaIO₄), sodium sulfite (Na₂SO₃), sodium borohydride (NaBH₄), deoxycholate (DOC), hydrochloric acid (HCl), sodium chloride (NaCl) [Merck], boric acid solution, phosphate buffered saline tablets (PBS) [Fluka], dithiothreitol (DTT) [Invitrogen].

4.2. Conjugation of GAC to CRM₁₉m Through Selective Direct Reductive Amination

The conjugation was performed as reported by Kabanova et al. [12]. Briefly, the reaction was carried out in 200 mM phosphate buffer (NaPi) at pH 8, with GAC concentration of 10 mg/mL, and a w/w/w ratio of GAC to CRM₁₉₇ to NaBH₃CN of 4:1:2. After 2 days at 37° C. the conjugate was purified by size exclusion chromatography on a 1.6×60 cm Sephacryl S-100 HR column (Cytiva Life Sciences, formerly GE Healthcare Life Sciences) eluted at 0.5 mL/minute in 10 mM NaPi pH 7.2. Final purified conjugate was designated as GAC-CRM₁₉₇.

4.3. Conjugation of GAC to Different Carrier Proteins Through Random Oxidation Followed by Reductive Amination

4.3.1. GAC Oxidation

After optimization of this step through DoE, GAC 1-10 mg/mL was oxidized with 8 mM NaIO₄ in borate at pH 8. The solution was kept at 25° C. in the dark, for 30 minutes. After that, NaIO₄ excess was quenched with 16 mM Na₂SO₃ in borate at pH 8. The mixture was gently stirred at room temperature (RT) for 15 minutes. The mixture was directly used for conjugation without intermediate purification or desalted through PD-10 Desalting column (Cytiva Life Sciences, formerly GE Healthcare Life Sciences). At higher scale, the purification was done by Tangential Flow Filtration (TFF). The TFF was performed with a Sartorius Hydrosart 10 kDa cut-off membrane with a 200 cm² membrane area. Fifteen volumes of diafiltration against water were performed (Pᵢₙ 1.0 bar, Pₒᵤₜ 0.0 bar, TMP 0.5 bar and permeate flow: 8-10 mL/min), keeping the retentate volume constant at 50 mL. The purified material, designated as GACox, was frozen at −80° C. and lyophilized.

4.3.2. Conjugation

GACox was conjugated to different carrier proteins (CRM₁₉₇, SLO, SpyAD, SpyCEP) in borate buffer at pH 8 in the presence of NaBH₃CN, with a GAC to protein to NaBH₃CN ratio of 4:1:2 w/w/w, and GACox concentration of 40 mg/mL. The reaction mixtures were incubated at 25° C. (for GAS proteins) or at 37° C. (for CRM₁₉₇) for 2 days. Conjugates of GAS proteins were purified by Amicon Ultra (Merck) kDa cut-off against 10 mM NaPi pH 7.2 (3500×g; 4° C.; 8 washes). CRM₁₉₇ conjugate was purified through anionic exchange chromatography on a 1 mL Sepharose Q FF column (Cytiva Life Sciences, formerly GE Healthcare Life Sciences): 1 mg of protein was loaded per mL of resin in 10 mM NaPi pH 7.2 and purified conjugate was eluted with a gradient of 1M NaCl. Collected fractions were dialyzed against 10 mM NaPi pH 7.2 buffer. Final purified conjugates were designated as GACox-proteins.

After DoE optimization, the conditions were changed as following: GACox 40 mg/mL with GACox to CRM₁₉₇ 1:1 w/w ratio, NaBH₃CN 5 mg/mL, borate pH 8, ON at 25° C. The reaction mixture was then diluted 10 times with PBS and NaBH₄ (NaBH₄:GAC w/w ratio of 0.5 to 1) was added to quench residual unreacted aldehydic groups of GACox [62]. The mixture was kept at RT for 2 h. Based on the scale, purification was done against PBS by Amicon Ultra 30 kDa cut-off as previously described, or by TFF. The TFF was performed with a Sartorius PESU 50 kDa cut-off membrane with a 200 cm² membrane area. Ten volumes of diafiltration against PBS 1M NaCl followed by 20 volumes of diafiltration against PBS alone were performed (Pᵢₙ 0.5 bar, Pₒᵤₜ 0.0 bar, TMP 0.25 bar, permeate flow rate: 25-27 mL/minutes), keeping the retentate volume constant at 50 mL.

4.4. Design of Experiment (DoE)

Experimental planning and data elaboration were performed with Design-Expert 10, Stat-Ease Inc. Anderson-Darling normality test was performed using Minitab 18, Minitab Inc.

For the oxidation step, each reaction test was performed on a total volume of 200 μL, purification was done through Vivaspin 3 kDa cut-off (Sartorius) against water. Oxidized GAC samples were assessed for % GAC recovery (based on Rha quantification by HPAEC-PAD), % GlcNAc oxidation, and for GAC average chain length by HPLC-SEC analysis.

For the conjugation reaction, GAC was oxidized at 10 mg/mL with 8 mM NaIO₄ in borate pH 8, for 30 minutes at 25° C. in the dark. After quenching of NaIO₄ excess, the mixture was desalted by PD 10 against water and split in different vials for the conjugation runs. All conjugations were performed on a total volume between 20 and 50 μL and purified via Amicon Ultra 30 kDa cut-off against 10 mM NaPi at pH 7.2. Conjugates were assessed for % GAC recovery, GAC/CRM₁₉₇ w/w ratios and % unconjugated CRM₁₉₇ in the mixture.

For both DoE, the analyses were done following the same randomization scheme used to carry out the reactions.

4.5. Analytical Methods

Oxidized GAC was characterized by HPAEC-PAD [63] for evaluating % of GlcNAc oxidized, by comparing GlcNAc to rhamnose (Rha) molar ratios before (start) and after (ox) oxidation. The following equation was used, with all concentrations expressed as pmol/mL: % GlcNAc oxidation=(1−([GlcNAcₒₓ]/(([GlcNAcₛₜₐᵣₜ]/[Rhaₛₜₐᵣₜ])*[Rhaₒₓ])))*100. HPLC-SEC was used for checking no changes in GAC chain length after oxidation.

Purified conjugates were characterized by micro BCA (Thermo Scientific) and HPAEC-PAD [63] for total protein and total GAC content respectively and to determine the PS to protein ratios in the final products. GAC concentration from HPAEC-PAD analysis was determined based on Rha quantification, as GlcNAc is impacted in the oxidation step. Free GAC was quantified by HPAEC-PAD after its separation from the conjugate by conjugate co-precipitation with DOC [64]. The reaction mixtures, as well as the purified conjugates, were analyzed by SDS-PAGE to compare protein patterns of the conjugates with corresponding unconjugated proteins, and by HPLC-SEC to verify conjugate formation (shift of the conjugate at higher MW compared to both unconjugated protein and saccharide). Finally, DSC analysis was used for evaluating GAS proteins and corresponding conjugates thermostability.

4.5.1. Sodium Dodecyl Sulfate-Polyacrylamide Gel Electrophoresis (SDS-PAGE)

Tris-acetate gels 7% (NuPAGE, from Invitrogen) were used for running SDS-PAGE analysis. The samples (5-20 μL with a protein content of 2-10 μg) were mixed with 0.5 M DTT (1/5, v/v) and NuPAGE LDS sample buffer (1/5, v/v). The mixtures were heated at 100° C. for 5 minutes. The gel, containing loaded samples, was electrophoresed at 45 mA in NuPAGE Tris-Acetate SDS running buffer (20×, Invitrogen) and stained with Coomassie® Blue Staining (Thermo Fischer).

4.5.2. High Performance Liquid Chromatography-Size Exclusion Chromatography (HPLC-SEC)

Conjugate, free protein and free GACox samples were eluted on a TSK gel G3000 PW_{XL} (30 cm×7.8 mm) column (particle size 7 μm) with TSK gel PW_{XL} guard column (4.0 cm×6.0 mm; particle size 12 μm) (TosohBioscience). The mobile phase was 0.1 M NaCl, 0.1 M NaH₂PO₄, 5% CH₃CN, pH 7.2 at the flow rate of 0.5 mL/minute (isocratic method for 35 min). Sample volume of injection was 80 μL. Void and bed volume calibration was performed with X-DNA (X-DNA Molecular Weight Marker Ill 0.12-21.2 Kbp, Roche) and sodium azide (NaN₃, Merck), respectively. GACox peaks were detected by refractive index (RI). Protein and conjugate peaks were also detected using tryptophan fluorescence (emission spectrum at 336 nm, with excitation wavelength at 280 nm). For the Kd determination the following equation was used: Kd=[(Te−T0)/(Tt−T0)] where: Te=elution time of the analyte, T0=elution time of the bigger fragment of λ-DNA and Tt=elution time of NaN₃.

4.5.3. Differential Scanning Calorimetry (DSC)

For DSC analysis the samples were prepared at a protein concentration of ˜2-3 μM in 10 mM NaPi at pH 7.2. The DSC temperature scan ranged from 10° C. to 110° C., with a thermal ramping rate of 150° C. per hour and a 5 second filter period. Data were analyzed by subtraction of the reference data for a sample containing buffer only. All experiments were performed in triplicate, and mean values of the melting temperature (Tm) were determined.

4.6. Immunogenicity Studies in Mice

Mouse studies were performed at the Toscana Life Sciences Animal Facility (Siena, Italy), in compliance with the relevant guidelines (Italian D. Lgs. n. 26/14 and European directive 2010/63/UE) and the institutional policies of GSK. The animal protocols were approved by the Animal Welfare Body of Toscana Life Sciences and by the Italian Ministry of Health (AEC project No. 201309 and GAS 734/2018-PR).

Female, 5 weeks old CD1 mice (8 per group) were vaccinated intraperitoneally (i.p.) with 200 μL of formulated antigens at study day 0 and 28. Approximately 100 μL bleeds (50 μL serum) were collected at day −1 (pooled sera) and at day 27 (individual sera) with final bleed at day 42.

Conjugates were formulated with 2 mg/mL Alhydrogel (Al³⁺). By SDS-PAGE silver staining analysis it was verified that >90% of the conjugates was adsorbed on Alhydrogel.

4.7. Assessment of Anti-GAC and Anti-GAS Carrier Protein Immune Responses in Mice

Pre-immune sera and individual mouse sera collected four weeks after the first and two weeks after the second immunization were analyzed for anti-GAC, -SpyCEP, -SLO and -SpyAD total IgG by enzyme-linked immunosorbent assay (ELISA) as previously described [65], with slight modifications. Briefly, mouse sera were diluted 1:100, 1:4000 and 1:160000 in PBS containing 0.05% Tween 20 and 0.1% BSA. ELISA units were expressed relative to mouse anti-antigen standard serum curves, with best 5 parameter fit determined by five-parameter logistic equation. One ELISA unit was defined as the reciprocal of the standard serum dilution that gives an absorbance value equal to 1 in the assay. Each mouse serum was run in triplicate. Data are presented as scatter plots of individual mouse ELISA units, and geometric mean of each group.

GAC-HSA (at the concentration of 1 μg/mL in carbonate buffer pH 9.6), SpyCEP, SLO and SpyAD (at the concentration of 2 μg/mL in carbonate buffer pH 9.6) were used as coating antigens.

4.8. Flow Cytometry (FACS)

GAS strain GAS51ΔM1 was grown overnight at 37° C., in the presence of 5% CO₂ in Todd Hewitt broth+Yeast extract (THY). Bacteria were pelleted at 8,000×g for 5 minutes and washed with PBS. Bacteria were then blocked with PBS containing 3% (w/v) BSA for 15 minutes and incubated with mouse sera diluted in PBS+1% (w/v) BSA (1:500, 1:5000 and 1:10000) for 1 hour. After washes with PBS, samples were incubated with Alexa Fluor 647 goat anti-mouse IgG (1:500) (Molecular Probes) for 30 minutes. Finally, bacteria were fixed with 4% (w/v) formaldehyde for 20 minutes and flow cytometry analysis was performed using FACS Canto II flow cytometer (BD Biosciences).

4.9. Functional Assays

4.9.1. IL-8 Cleavage Inhibition Assay

Pre-immune and post-second immunization individual sera were tested in an IL-8 cleavage ELISA assay to evaluate their ability to block SpyCEP proteolytic activity. The assay was performed as previously described [14] with some modifications. Briefly, SpyCEP (5 ng/mL) was preincubated with mouse polyclonal anti-SpyCEP serum at four different dilutions (1:100, 1:300, 1:900, 1:2700) for 5 minutes at 4° C. in PBS 0.5 mg/ml BSA. Pre-incubation of SpyCEP with buffer only and with pre-immune serum were used as negative controls. Then, human IL-8 (Gibco, 10 ng/ml) was added and the reaction was incubated at 37° C. (reaction without enzyme was used as control). After 2 hours, each reaction mix was diluted 20-fold and incubated in 96-well plates coated with a blend of monoclonal antibodies directed against distinct epitopes of IL-8 (Life Technologies). The amount of IL-8 in each sample and in the control reaction (without the enzyme) was determined according to the manufacturer's protocols, using a standard curve of IL-8. Each serum dilution was tested twice, and the mean value with error bar was reported in the graph. Results are expressed as amount (ng/mL) of uncleaved IL-8 at each serum concentration tested.

4.9.2. In Vitro Hemolysis Assay

Pre-immune and post-second immunization individual sera were tested in a hemolysis assay to evaluate their ability to block SLO hemolytic activity. The assay was performed as previously described [14] with some modifications. Briefly, a red blood cell suspension was prepared by washing rabbit red blood cells (Emozoo) four times in PBS and resuspended in PBS (20% rabbit red blood cell suspension in PBS). Eight serial 2-fold dilutions of either anti-SLO sera or negative control pre-immune serum diluted in PBS with 0.5% BSA were prepared in 96 well round bottom plates then preincubated with 900 units/mL of SLO toxin (Sigma, diluted in PBS with 15 mM dithiothreitol, Invitrogen) at RT for 30 minutes (in a final volume of 150 μL). Following addition of rabbit blood cell suspension (50 μL), incubation was continued for 30 minutes at 37° C. Plates were finally centrifuged for 5 minutes at 1000×g and the supernatant was carefully transferred to 96-well flat-bottomed plates. The absorbance of the released hemoglobin was read at 540 nm. Each serum dilution was tested twice, and the mean value with error bar was reported in the graph. Results are expressed as amount of hemoglobin (OD540) released by rabbit red blood cells at each serum concentration tested.

4.10. Statistics

Mann-Whitney two-tailed test was used to compare the immune response elicited by two different antigens, Kruskal-Wallis test with Dunn's post hoc analysis was used for comparison among more than two groups. Wilcoxon test matched-pairs signed rank two-tailed test was performed to compare the response induced by the same antigen at day 27 vs day 42.

### Abbreviations

GAS Group A Streptococcus
GAC Group A Carbohydrate
SLO Streptolysin O
LMICs Low- and Middle-income Countries
RHD Rheumatic Hearth Disease
GlcNAc N-acetylglucosamine
PS Polysaccharide
SDS-PAGE Sodium dodecyl sulfate-polyacrylamide gel electrophoresis
MW Molecular Weight
HPLC-SEC High Performance Liquid Chromatography-Size Exclusion Chromatography
i.p. Intraperitoneally
DSC Differential Scanning Calorimetry
FACS Flow cytometry
DoE Design of Experiment
RT Room Temperature
Rha Rhamnose
HPAEC-PAD Anion Exchange Chromatography coupled with Pulsed Amperometric Detection
TFF Tangential Flow Filtration
ELISA Enzyme-linked immunosorbent assay

### 5. Numbered References

1. Ralph, A. P.; Carapetis, J. R., Group a streptococcal diseases and their global burden. Curr Top Microbiol Immunol 2013, 368, 1-27.
2. Carapetis, J. R.; Steer, A. C.; Mulholland, E. K.; Weber, M., The global burden of group A streptococcal diseases. Lancet Infect Dis 2005, 5, (11), 685-94.
3. Dooling, K. L.; Shapiro, D. J.; Van Beneden, C.; Hersh, A. L.; Hicks, L. A., Overprescribing and inappropriate antibiotic selection for children with pharyngitis in the United States, 1997-2010. JAMA Pediatr 2014, 168, (11), 1073-4.
4. Jansen, K. U.; Knirsch, C.; Anderson, A. S., The role of vaccines in preventing bacterial antimicrobial resistance. Nature medicine 2018, 24, (1), 10-19.
5. Watkins, D. A.; Johnson, C. O.; Colquhoun, S. M.; Karthikeyan, G.; Beaton, A.; Bukhman, G.; Forouzanfar, M. H.; Longenecker, C. T.; Mayosi, B. M.; Mensah, G. A.; Nascimento, B. R.; Ribeiro, A. L. P.; Sable, C. A.; Steer, A. C.; Naghavi, M.; Mokdad, A. H.; Murray, C. J. L.; Vos, T.; Carapetis, J. R.; Roth, G. A., Global, Regional, and National Burden of Rheumatic Heart Disease, 1990-2015. N Engl J Med 2017, 377, (8), 713-722.
6. Vekemans, J.; Gouvea-Reis, F.; Kim, J. H.; Excler, J. L.; Smeesters, P. R.; O'Brien, K. L.; Van Beneden, C. A.; Steer, A. C.; Carapetis, J. R.; Kaslow, D. C., The Path to Group A Streptococcus Vaccines: World Health Organization Research and Development Technology Roadmap and Preferred Product Characteristics. Clin Infect Dis 2019, 69, (5), 877-883.
7. Walker, M. J.; Barnett, T. C.; McArthur, J. D.; Cole, J. N.; Gillen, C. M.; Henningham, A.; Sriprakash, K. S.; Sanderson-Smith, M. L.; Nizet, V., Disease manifestations and pathogenic mechanisms of Group A Streptococcus. Clin Microbiol Rev 2014, 27, (2), 264-301.
8. Schneerson, R.; Barrera, O.; Sutton, A.; Robbins, J. B., Preparation, characterization, and immunogenicity of Haemophilus influenzae type b polysaccharide-protein conjugates. J Exp Med 1980, 152, (2), 361-76.
9. Berti, F.; Micoli, F., Improving efficacy of glycoconjugate vaccines: from chemical conjugates to next generation constructs. Curr Opin Immunol 2020, 65, 42-49.
10. Rappuoli, R., Glycoconjugate vaccines: Principles and mechanisms. Sci Trans/Med 2018, 10, (456).
11. Salvadori, L. G.; Blake, M. S.; McCarty, M.; Tai, J. Y.; Zabriskie, J. B., Group A streptococcus-liposome ELISA antibody titers to group A polysaccharide and opsonophagocytic capabilities of the antibodies. J Infect Dis 1995, 171, (3), 593-600.
12. Kabanova, A.; Margarit, I.; Berti, F.; Romano, M. R.; Grandi, G.; Bensi, G.; Chiarot, E.; Proietti, D.; Swennen, E.; Cappelletti, E.; Fontani, P.; Casini, D.; Adamo, R.; Pinto, V.; Skibinski, D.; Capo, S.; Buffi, G.; Gallotta, M.; Christ, W. J.; Campbell, A. S.; Pena, J.; Seeberger, P. H.; Rappuoli, R.; Costantino, P., Evaluation of a Group A Streptococcus synthetic oligosaccharide as vaccine candidate. Vaccine 2010, 29, (1), 104-14.
13. Sabharwal, H.; Michon, F.; Nelson, D.; Dong, W.; Fuchs, K.; Manjarrez, R. C.; Sarkar, A.; Uitz, C.; Viteri-Jackson, A.; Suarez, R. S.; Blake, M.; Zabriskie, J. B., Group A streptococcus (GAS) carbohydrate as an immunogen for protection against GAS infection. J Infect Dis 2006, 193, (1), 129-35.
14. Bensi, G.; Mora, M.; Tuscano, G.; Biagini, M.; Chiarot, E.; Bombaci, M.; Capo, S.; Falugi, F.; Manetti, A. G.; Donato, P.; Swennen, E.; Gallotta, M.; Garibaldi, M.; Pinto, V.; Chiappini, N.; Musser, J. M.; Janulczyk, R.; Mariani, M.; Scarselli, M.; Telford, J. L.; Grifantini, R.; Norais, N.; Margarit, I.; Grandi, G., Multi high-throughput approach for highly selective identification of vaccine candidates: the Group A Streptococcus case. Mol Cell Proteomics 2012, 11, (6), M111 015693.
15. Cunningham, M. W., Pathogenesis of group A streptococcal infections. Clin Microbiol Rev 2000, 13, (3), 470-511.
16. Uchiyama, S.; Dohrmann, S.; Timmer, A. M.; Dixit, N.; Ghochani, M.; Bhandari, T.; Timmer, J. C.; Sprague, K.; Bubeck-Wardenburg, J.; Simon, S. I.; Nizet, V., Streptolysin O Rapidly Impairs Neutrophil Oxidative Burst and Antibacterial Responses to Group A Streptococcus. Front Immunol 2015, 6, 581.
17. Gallotta, M.; Gancitano, G.; Pietrocola, G.; Mora, M.; Pezzicoli, A.; Tuscano, G.; Chiarot, E.; Nardi-Dei, V.; Taddei, A. R.; Rindi, S.; Speziale, P.; Soriani, M.; Grandi, G.; Margarit, I.; Bensi, G., SpyAD, a moonlighting protein of group A Streptococcus contributing to bacterial division and host cell adhesion. Infect Immun 2014, 82, (7), 2890-901.
18. Edwards, R. J.; Taylor, G. W.; Ferguson, M.; Murray, S.; Rendell, N.; Wrigley, A.; Bai, Z.; Boyle, J.; Finney, S. J.; Jones, A.; Russell, H. H.; Turner, C.; Cohen, J.; Faulkner, L.; Sriskandan, S., Specific C-terminal cleavage and inactivation of interleukin-8 by invasive disease isolates of Streptococcus pyogenes. J Infect Dis 2005, 192, (5), 783-90.
19. Jobichen, C.; Tan, Y. C.; Prabhakar, M. T.; Nayak, D.; Biswas, D.; Pannu, N. S.; Hanski, E.; Sivaraman, J., Structure of ScpC, a virulence protease from Streptococcus pyogenes, reveals the functional domains and maturation mechanism. Biochem J 2018, 475, (17), 2847-2860.
20. Davies, M. R.; McIntyre, L.; Mutreja, A.; Lacey, J. A.; Lees, J. A.; Towers, R. J.; Duchene, S.; Smeesters, P. R.; Frost, H. R.; Price, D. J.; Holden, M. T. G.; David, S.; Giffard, P. M.; Worthing, K. A.; Seale, A. C.; Berkley, J. A.; Harris, S. R.; Rivera-Hernandez, T.; Berking, O.; Cork, A. J.; Torres, R.; Lithgow, T.; Strugnell, R. A.; Bergmann, R.; Nitsche-Schmitz, P.; Chhatwal, G. S.; Bentley, S. D.; Fraser, J. D.; Moreland, N. J.; Carapetis, J. R.; Steer, A. C.; Parkhill, J.; Saul, A.; Williamson, D. A.; Currie, B. J.; Tong, S. Y. C.; Dougan, G.; Walker, M. J., Atlas of group A streptococcal vaccine candidates compiled using large-scale comparative genomics. Nat Genet 2019, 51, (6), 1035-1043.
21. Micoli, F.; Adamo, R.; Costantino, P., Protein Carriers for Glycoconjugate Vaccines: History, Selection Criteria, Characterization and New Trends. Molecules 2018, 23, (6).
22. Tontini, M.; Romano, M. R.; Proietti, D.; Balducci, E.; Micoli, F.; Balocchi, C.; Santini, L.; Masignani, V.; Berti, F.; Costantino, P., Preclinical studies on new proteins as carrier for glycoconjugate vaccines. Vaccine 2016, 34, (35), 4235-4242.
23. Roy, R.; Katzenellenbogen, E.; Jennings, H. J., Improved procedures for the conjugation of oligosaccharides to protein by reductive amination. Can J Biochem Cell Biol 1984, 62, (5), 270-5.
24. Steer, A. C.; Law, I.; Matatolu, L.; Beall, B. W.; Carapetis, J. R., Global emm type distribution of group A streptococci: systematic review and implications for vaccine development. Lancet Infect Dis 2009, 9, (10), 611-6.
25. Dale, J. B.; Penfound, T. A.; Chiang, E. Y.; Walton, W. J., New 30-valent M protein-based vaccine evokes cross-opsonic antibodies against non-vaccine serotypes of group A streptococci. Vaccine 2011, 29, (46), 8175-8.
26. Postol, E.; Alencar, R.; Higa, F. T.; Freschi de Barros, S.; Demarchi, L. M.; Kalil, J.; Guilherme, L., StreptlnCor: a candidate vaccine epitope against S. pyogenes infections induces protection in outbred mice. PLoS One 2013, 8, (4), e60969.
27. Sekuloski, S.; Batzloff, M. R.; Griffin, P.; Parsonage, W.; Elliott, S.; Hartas, J.; O'Rourke, P.; Marquart, L.; Pandey, M.; Rubin, F. A.; Carapetis, J.; McCarthy, J.; Good, M. F., Evaluation of safety and immunogenicity of a group A streptococcus vaccine candidate (MJ8VAX) in a randomized clinical trial. PLoS One 2018, 13, (7), e0198658.
28. Steer, A. C.; Carapetis, J. R.; Dale, J. B.; Fraser, J. D.; Good, M. F.; Guilherme, L.; Moreland, N. J.; Mulholland, E. K.; Schodel, F.; Smeesters, P. R., Status of research and development of vaccines for Streptococcus pyogenes. Vaccine 2016, 34, (26), 2953-2958.
29. Avci, F.; Berti, F.; Dull, P.; Hennessey, J.; Pavliak, V.; Prasad, A. K.; Vann, W.; Wacker, M.; Marcq, O., Glycoconjugates: What It Would Take To Master These Well-Known yet Little-Understood Immunogens for Vaccine Development. mSphere 2019, 4, (5).
30. Dagan, R.; Poolman, J.; Siegrist, C. A., Glycoconjugate vaccines and immune interference: A review. Vaccine 2010, 28, (34), 5513-23.
31. Michon, F.; Fusco, P. C.; Minetti, C. A.; Laude-Sharp, M.; Uitz, C.; Huang, C. H.; D'Ambra, A. J.; Moore, S.; Remeta, D. P.; Heron, I.; Blake, M. S., Multivalent pneumococcal capsular polysaccharide conjugate vaccines employing genetically detoxified pneumolysin as a carrier protein. Vaccine 1998, 16, (18), 1732-41.
32. Nilo, A.; Morelli, L.; Passalacqua, I.; Brogioni, B.; Allan, M.; Carboni, F.; Pezzicoli, A.; Zerbini, F.; Maione, D.; Fabbrini, M.; Romano, M. R.; Hu, Q. Y.; Margarit, I.; Berti, F.; Adamo, R., Anti-Group B Streptococcus Glycan-Conjugate Vaccines Using Pilus Protein GBS80 As Carrier and Antigen: Comparing Lysine and Tyrosine-directed Conjugation. ACS Chem Biol 2015, 10, (7), 1737-46.
33. Nilo, A.; Passalacqua, I.; Fabbrini, M.; Allan, M.; Usera, A.; Carboni, F.; Brogioni, B.; Pezzicoli, A.; Cobb, J.; Romano, M. R.; Margarit, I.; Hu, Q. Y.; Berti, F.; Adamo, R., Exploring the Effect of Conjugation Site and Chemistry on the Immunogenicity of an anti-Group B Streptococcus Glycoconjugate Vaccine Based on GBS67 Pilus Protein and Type V Polysaccharide. Bioconjug Chem 2015, 26, (8), 1839-49.
34. Pinto, V. B.; Burden, R.; Wagner, A.; Moran, E. E.; Lee, C. H., The development of an experimental multiple serogroups vaccine for Neisseria meningitidis. PLoS One 2013, 8, (11), e79304.
35. Pozzi, C.; Wilk, K.; Lee, J. C.; Gening, M.; Nifantiev, N.; Pier, G. B., Opsonic and protective properties of antibodies raised to conjugate vaccines targeting six Staphylococcus aureus antigens. PLoS One 2012, 7, (10), e46648.
36. Simon, R.; Tennant, S. M.; Wang, J. Y.; Schmidlein, P. J.; Lees, A.; Ernst, R. K.; Pasetti, M. F.; Galen, J. E.; Levine, M. M., Salmonella enterica serovar enteritidis core O polysaccharide conjugated to H:g,m flagellin as a candidate vaccine for protection against invasive infection with S. enteritidis. Infect Immun 2011, 79, (10), 4240-9.
37. Rivera-Hernandez, T.; Pandey, M.; Henningham, A.; Cole, J.; Choudhury, B.; Cork, A. J.; Gillen, C. M.; Ghaffar, K. A.; West, N. P.; Silvestri, G.; Good, M. F.; Moyle, P. M.; Toth, I.; Nizet, V.; Batzloff, M. R.; Walker, M. J., Differing Efficacies of Lead Group A Streptococcal Vaccine Candidates and Full-Length M Protein in Cutaneous and Invasive Disease Models. mBio 2016, 7, (3).
38. Wang, S.; Zhao, Y.; Wang, G.; Feng, S.; Guo, Z.; Gu, G., Group A Streptococcus Cell Wall Oligosaccharide-Streptococcal C5a Peptidase Conjugates as Effective Antibacterial Vaccines. ACS infectious diseases 2020, 6, (2), 281-290.
39. Zhao, Y.; Wang, S.; Wang, G.; Li, H.; Guo, Z.; Gu, G., Synthesis and immunological studies of group A Streptococcus cell-wall oligosaccharide-streptococcal C5a peptidase conjugates as bivalent vaccines. Organic Chemistry Frontiers 2019, 6, (20), 3589-3596.
40. Chiarot, E.; Faralla, C.; Chiappini, N.; Tuscano, G.; Falugi, F.; Gambellini, G.; Taddei, A.; Capo, S.; Cartocci, E.; Veggi, D.; Corrado, A.; Mangiavacchi, S.; Tavarini, S.; Scarselli, M.; Janulczyk, R.; Grandi, G.; Margarit, I.; Bensi, G., Targeted amino acid substitutions impair streptolysin O toxicity and group A Streptococcus virulence. mBio 2013, 4, (1), e00387-12.
41. McKenna, S.; Malito, E.; Rouse, S. L.; Abate, F.; Bensi, G.; Chiarot, E.; Micoli, F.; Mancini, F.; Gomes Moriel, D.; Grandi, G.; Mossakowska, D.; Pearson, M.; Xu, Y.; Pease, J.; Sriskandan, S.; Margarit, I.; Bottomley, M. J.; Matthews, S., Structure, dynamics and immunogenicity of a catalytically inactive CXC chemokine-degrading protease SpyCEP from Streptococcus pyogenes. Computational and structural biotechnology journal 2020, 18, 650-660.
42. Stefanetti, G.; Hu, Q. Y.; Usera, A.; Robinson, Z.; Allan, M.; Singh, A.; Imase, H.; Cobb, J.; Zhai, H.; Quinn, D.; Lei, M.; Saul, A.; Adamo, R.; MacLennan, C. A.; Micoli, F., Sugar-Protein Connectivity Impacts on the Immunogenicity of Site-Selective Salmonella O-Antigen Glycoconjugate Vaccines. Angew Chem Int Ed Eng/2015, 54, (45), 13198-203.
43. Wacker, M.; Linton, D.; Hitchen, P. G.; Nita-Lazar, M.; Haslam, S. M.; North, S. J.; Panico, M.; Morris, H. R.; Dell, A.; Wren, B. W.; Aebi, M., N-linked glycosylation in Campylobacterjejuni and its functional transfer into E. coli. Science (New York, N.Y.) 2002, 298, (5599), 1790-3.
44. Wacker, M.; Wang, L.; Kowarik, M.; Dowd, M.; Lipowsky, G.; Faridmoayer, A.; Shields, K.; Park, S.; Alaimo, C.; Kelley, K. A.; Braun, M.; Quebatte, J.; Gambillara, V.; Carranza, P.; Steffen, M.; Lee, J. C., Prevention of Staphylococcus aureus infections by glycoprotein vaccines synthesized in Escherichia coli. J Infect Dis 2014, 209, (10), 1551-61.
45. Carmenate, T.; Canaan, L.; Alvarez, A.; Delgado, M.; Gonzalez, S.; Menendez, T.; Rodes, L.; Guillen, G., Effect of conjugation methodology on the immunogenicity and protective efficacy of meningococcal group C polysaccharide-P64k protein conjugates. FEMS Immunol Med Microbiol 2004, 40, (3), 193-9.
46. Costantino, P.; Rappuoli, R.; Berti, F., The design of semi-synthetic and synthetic glycoconjugate vaccines. Expert Opin Drug Discov 2011, 6, (10), 1045-66.
47. Fattom, A.; Li, X.; Cho, Y. H.; Burns, A.; Hawwari, A.; Shepherd, S. E.; Coughlin, R.; Winston, S.; Naso, R., Effect of conjugation methodology, carrier protein, and adjuvants on the immune response to Staphylococcus aureus capsular polysaccharides. Vaccine 1995, 13, (14), 1288-93.
48. Rush, J. S.; Edgar, R. J.; Deng, P.; Chen, J.; Zhu, H.; van Sorge, N. M.; Morris, A. J.; Korotkov, K. V.; Korotkova, N., The molecular mechanism of N-acetylglucosamine side-chain attachment to the Lancefield group A carbohydrate in Streptococcus pyogenes. J Biol Chem 2017, 292, (47), 19441-19457.
49. Duan, J.; Kasper, D. L., Oxidative depolymerization of polysaccharides by reactive oxygen/nitrogen species. Glycobiology 2011, 21, (4), 401-9.
50. Kholy, A. E.; Facklam, R.; Sabri, G.; Rotta, J., Serological identification of group A streptococci from throat scrapings before culture. J Clin Microbiol 1978, 8, (6), 725-8.
51. Pancholi, V.; Fischetti, V. A., Isolation and characterization of the cell-associated region of group A streptococcal M6 protein. J Bacteriol 1988, 170, (6), 2618-24.
52. Beg, S.; Swain, S.; Rahman, M.; Hasnain, M. S.; Imam, S. S., Chapter 3—Application of Design of Experiments (DoE) in Pharmaceutical Product and Process Optimization. In Pharmaceutical Quality by Design, Beg, S.; Hasnain, M. S., Eds. Academic Press: 2019; pp 43-64.
53. Montgomery, D. C., Response surface methods and other approaches to process optimization. Design and analysis of experiments 1997.
54. Giannelli, C.; Raso, M. M.; Palmieri, E.; De Felice, A.; Pippi, F.; Micoli, F., Development of a Specific and Sensitive HPAEC-PAD Method for Quantification of Vi Polysaccharide Applicable to other Polysaccharides Containing Amino Uronic Acids. Anal Chem 2020, 92, (9), 6304-6311.
55. Joelsson, D.; Moravec, P.; Troutman, M.; Pigeon, J.; DePhillips, P., Optimizing ELISAs for precision and robustness using laboratory automation and statistical design of experiments. J Immunol Methods 2008, 337, (1), 35-41.
56. Necchi, F.; Carducci, M.; Pisoni, I.; Rossi, O.; Saul, A.; Rondini, S., Development of FAcE (Formulated Alhydrogel competitive ELISA) method for direct quantification of OAg present in Shigella sonnei GMMA-based vaccine and its optimization using Design of Experiments approach. J Immunol Methods 2019, 471, 11-17.
57. Ahl, P. L.; Mensch, C.; Hu, B.; Pixley, H.; Zhang, L.; Dieter, L.; Russell, R.; Smith, W. J.; Przysiecki, C.; Kosinski, M.; Blue, J. T., Accelerating Vaccine Formulation Development Using Design of Experiment Stability Studies. Journal of pharmaceutical sciences 2016, 105, (10), 3046-3056.
58. Ji, Y.; Tian, Y.; Ahnfelt, M.; Sui, L., Design and optimization of a chromatographic purification process for Streptococcus pneumoniae serotype 23F capsular polysaccharide by a Design of Experiments approach. Journal of chromatography. A 2014, 1348, 137-49.
59. Kanojia, G.; Willems, G. J.; Frijlink, H. W.; Kersten, G. F.; Soema, P. C.; Amorij, J. P., A Design of Experiment approach to predict product and process parameters for a spray dried influenza vaccine. International journal of pharmaceutics 2016, 511, (2), 1098-111.
60. Patel, A.; Erb, S. M.; Strange, L.; Shukla, R. S.; Kumru, 0. S.; Smith, L.; Nelson, P.; Joshi, S. B.; Livengood, J. A.; Volkin, D. B., Combined semi-empirical screening and design of experiments (DOE) approach to identify candidate formulations of a lyophilized live attenuated tetravalent viral vaccine candidate. Vaccine 2018, 36, (22), 3169-3179.
61. Abate, F.; Malito, E.; Falugi, F.; Margarit, Y. R. I.; Bottomley, M. J., Cloning, expression, purification, crystallization and preliminary X-ray diffraction analysis of SpyCEP, a candidate antigen for a vaccine against Streptococcus pyogenes. Acta Crystallogr Sect F Struct Biol Cryst Commun 2013, 69, (Pt 10), 1103-6.
62. Stefanetti, G.; Rondini, S.; Lanzilao, L.; Saul, A.; MacLennan, C. A.; Micoli, F., Impact of conjugation chemistry on the immunogenicity of S. typhimurium conjugate vaccines. Vaccine 2014, 32, (46), 6122-9.
63. Pitirollo, O.; Micoli, F.; Necchi, F.; Mancini, F.; Carducci, M.; Adamo, R.; Evangelisti, C.; Morelli, L.; Polito, L.; Lay, L., Gold nanoparticles morphology does not affect the multivalent presentation and antibody recognition of Group A Streptococcus synthetic oligorhamnans. Bioorg Chem 2020, 99, 103815.
64. Lei, Q. P.; Lamb, D. H.; Heller, R.; Pietrobon, P., Quantitation of low level unconjugated polysaccharide in tetanus toxoid-conjugate vaccine by HPAEC/PAD following rapid separation by deoxycholate/HCl. J Pharm Biomed Anal 2000, 21, (6), 1087-91.
65. Lanzilao, L.; Stefanetti, G.; Saul, A.; MacLennan, C. A.; Micoli, F.; Rondini, S., Strain Selection for Generation of O-Antigen-Based Glycoconjugate Vaccines against Invasive Nontyphoidal Salmonella Disease. PLoS One 2015, 10, (10), e0139847.

### 6. Supplementary Tables

TABLE S1
DoE approach applied to GAC oxidation: summary of
conditions tested and results obtained.

| | | Factor 1 | Factor 2 | Factor | Response 1 | Response 2 | Response |
|---|---|---|---|---|---|---|---|
| | | A:[PS] | B:[NalO₄] | 3 | Recovery | Oxidation | 3 Size |
| Std | Run | mg/ml | mM | C:pH | % | GlcNAc % | Da |
| 5 | 1 | 2.8 | 2.4 | 7.4 | 62 | 10.2 | 6970 |
| 1 | 2 | 2.8 | 2.4 | 5.6 | 69 | 13.5 | 6970 |
| 20 | 3 | 5.5 | 5.3 | 6.5 | 62 | 11.9 | 6782 |
| 15 | 4 | 5.5 | 5.3 | 6.5 | 76 | 12.7 | 6766 |
| 2 | 5 | 8.2 | 2.4 | 5.6 | 87 | 10.3 | 6886 |
| 6 | 6 | 8.2 | 2.4 | 7.4 | 78 | 8.5 | 6927 |
| 16 | 7 | 5.5 | 5.3 | 6.5 | 80 | 12.2 | 6807 |
| 12 | 8 | 5.5 | 10.0 | 6.5 | 88 | 16.7 | 6625 |
| 4 | 9 | 8.2 | 8.0 | 5.6 | 93 | 13.3 | 6825 |
| 17 | 10 | 5.5 | 5.3 | 6.5 | 78 | 12.8 | 6784 |
| 14 | 11 | 5.5 | 5.3 | 8.0 | 82 | 11.8 | 6808 |
| 7 | 12 | 2.8 | 8.0 | 7.4 | 77 | 14.4 | 6764 |
| 3 | 13 | 2.8 | 8.0 | 5.6 | 72 | 19.2 | 6766 |
| 9 | 14 | 1.0 | 5.3 | 6.5 | 70 | 19.4 | 6447 |
| 11 | 15 | 5.5 | 0.5 | 6.5 | 77 | nd | 7028 |
| 8 | 16 | 8.2 | 8.0 | 7.4 | 80 | 17.8 | 6726 |
| 13 | 17 | 5.5 | 5.3 | 5.0 | 84 | 17.8 | 6825 |
| 18 | 18 | 5.5 | 5.3 | 6.5 | 76 | 14.9 | 6774 |
| 10 | 19 | 10.0 | 5.3 | 6.5 | 88 | 15.1 | 6835 |
| 19 | 20 | 5.5 | 5.3 | 6.5 | 88 | nd | 6694 |

TABLE S2
DoE approach applied to conjugation of GACox to CRM197: summary of
conditions tested and results obtained.

| | | | | | Response | Response | Response 4 |
|---|---|---|---|---|---|---|---|
| | | | | | 2 | 3 | unconjugated |
| | | Factor 1 | Factor 2 | Factor 3 | w/w ratio | Recovered | CRM₁₉₇ in |
| | | A:[GACox] | B:[CRM₁₉₇] | C:[NaBH₃CN] | GAC/ | PS | mixture |
| Std | Run | mg/ml | mg/ml | mg/ml | CRM₁₉₇ | % | % |
| 20 | 1 | 25 | 25 | 25 | 0.26 | 21.1 | 0 |
| 8 | 2 | 40 | 40 | 40 | 0.28 | 20.7 | 0 |
| 17 | 3 | 25 | 25 | 25 | 0.29 | 23.1 | 0 |
| 15 | 4 | 25 | 25 | 25 | 0.29 | 22.6 | 0 |
| 13 | 5 | 25 | 25 | 10 | 0.42 | 35.4 | 0 |
| 6 | 6 | 40 | 10 | 40 | 0.49 | 9.4 | 0 |
| 5 | 7 | 10 | 10 | 40 | 0.16 | 12.8 | 0 |
| 4 | 8 | 40 | 40 | 10 | 0.43 | 41.3 | 0 |
| 3 | 9 | 10 | 40 | 10 | 0.12 | 41.2 | 33 |
| 12 | 10 | 25 | 40 | 25 | 0.34 | 41.9 | 0 |
| 16 | 11 | 25 | 25 | 25 | 0.30 | 22.2 | 0 |
| 14 | 12 | 25 | 25 | 40 | 0.25 | 18.9 | 0 |
| 9 | 13 | 10 | 25 | 25 | 0.15 | 32.9 | 0 |
| 1 | 14 | 10 | 10 | 10 | 0.27 | 22.0 | 5 |
| 10 | 15 | 40 | 25 | 25 | 0.49 | 25.6 | 3 |
| 11 | 16 | 25 | 10 | 25 | 0.47 | 12.5 | 0 |
| 2 | 17 | 40 | 10 | 10 | 0.65 | 9.2 | 16 |
| 18 | 18 | 25 | 25 | 25 | 0.33 | 26.1 | 0 |
| 7 | 19 | 10 | 40 | 40 | 0.12 | 30.8 | 11 |
| 19 | 20 | 25 | 25 | 25 | 0.34 | 27.0 | 0 |

TABLE S3
Identification of optimal conditions for GAC oxidation:
statistical analysis of the model for the DoE.
ANOVA for Response Surface Reduced Quadratic model
Analysis of variance table [Partial sum of squares-Type III]

| | Sum of | | Mean | F | p-value | |
|---|---|---|---|---|---|---|
| Source | Squares | df | Square | Value | Prob > F | |
| Model | 134.14 | 5 | 26.83 | 9.33 | 0.0008 | significant |
| A-[PS] | 15.35 | 1 | 15.35 | 5.34 | 0.0395 | |
| B-[NalO4] | 73.85 | 1 | 73.85 | 25.67 | 0.0003 | |
| C-pH | 17.52 | 1 | 17.52 | 6.09 | 0.0296 | |
| AC | 14.61 | 1 | 14.61 | 5.08 | 0.0437 | |
| A² | 19.97 | 1 | 19.97 | 6.94 | 0.0218 | |
| Residual | 34.52 | 12 | 2.88 | | | |
| Lack of Fit | 29.07 | 8 | 3.63 | 2.66 | 0.1798 | not |
| | | | | | | significant |
| Pure Error | 5.46 | 4 | 1.36 | | | |
| Cor Total | 168.67 | 17 | | | | |

| | | | |
|---|---|---|---|
| Std. Dev. | 1.70 | R-Squared | 0.7953 |
| Mean | 14.04 | Adj R-Squared | 0.7100 |
| C.V. % | 12.08 | Pred R-Squared | 0.4656 |
| PRESS | 90.14 | Adeq Precision | 10.476 |
| −2 Log | 62.80 | BIC | 80.15 |
| Likelihood | | AICc | 82.44 |

| | Coefficient | | Standard | 95% CI | 95% CI | |
|---|---|---|---|---|---|---|
| Factor | Estimate | df | Error | Low | High | VIF |
| Intercept | 12.87 | 1 | 0.54 | 11.70 | 14.04 | |
| A-[PS] | −1.06 | 1 | 0.46 | −2.06 | −0.060 | 1.00 |
| B-[NalO4] | 2.65 | 1 | 0.52 | 1.51 | 3.78 | 1.01 |
| C-pH | −1.13 | 1 | 0.46 | −2.13 | −0.13 | 1.00 |
| AC | 1.35 | 1 | 0.60 | 0.045 | 2.66 | 1.00 |
| A² | 1.22 | 1 | 0.46 | 0.21 | 2.22 | 1.01 |

TABLE S4
Identification of optimal conditions for GACox conjugation to CRM 197: statistical
analysis of the models for GAC/CRM197 w/w ratio (a) and GAC yield (b).
(a) Response w/w ratio GAC/CRM₁₉₇
ANOVA for Response Surface Linear model
Analysis of variance table [Partial sum of squares-Type III]

| | Sum of | | Mean | F | p-value | |
|---|---|---|---|---|---|---|
| Source | Squares | df | Square | Value | Prob > F | |
| Model | 0.32 | 3 | 0.11 | 43.45 | <0.0001 | significant |
| A-[GAC] | 0.23 | 1 | 0.23 | 94.44 | <0.0001 | |
| B-[CRM₁₉₇] | 0.056 | 7 | 0.056 | 22.64 | 0.0002 | |
| C- | 0.033 | 1 | 0.033 | 13.28 | 0.0022 | |
| [NaBH₃CN] | | | | | | |
| Residual | 0.039 | 16 | 2.455E−003 | | | |
| Lack of Fit | 0.035 | 11 | 3.153E−003 | 3.42 | 0.0926 | not |
| | | | | | | significant |
| Pure Error | 4.608E−003 | 5 | 9.216E−004 | | | |
| Cor Total | 0.36 | 19 | | | | |

| | | | |
|---|---|---|---|
| Std. Dev. | 0.050 | R-Squared | 0.8907 |
| Mean | 0.32 | Adj R-Squared | 0.8702 |
| C.V. % | 15.32 | Pred R-Squared | 0.8084 |
| PRESS | 0.069 | Adeq Precision | 25.625 |
| −2 Log Likelihood | −67.89 | BIC | −55.91 |
| | | AICc | −57.23 |

| | Coefficient | | Standard | 95% CI | 95% CI | |
|---|---|---|---|---|---|---|
| Factor | Estimate | df | Error | Low | High | VIF |
| Intercept | 0.32 | 1 | 0.011 | 0.30 | 0.35 | |
| A-[GAC] | 0.15 | 1 | 0.016 | 0.12 | 0.19 | 1.00 |
| B-[CRM-₁₉₇] | −0.075 | 1 | 0.016 | −0.11 | −0.041 | 1.00 |
| C- | −0.057 | 1 | 0.016 | −0.090 | −0.024 | 1.00 |
| [NaBH₃CN] | | | | | | |

(b) Response Recovered PS %
ANOVA for Response Surface Linear model
Analysis of table [Partial sum of squares-Type III]

| | Sum of | | Mean | F | p-value | |
|---|---|---|---|---|---|---|
| Source | Squares | df | Square | Value | Prob > F | |
| Model | 1640.06 | 3 | 546.69 | 31.69 | <0.0001 | significant |
| A-[GAC] | 112.07 | 1 | 112.07 | 6.50 | 0.0215 | |
| B-[CRM₁₉₇] | 1209.63 | 1 | 1209.63 | 70.11 | <0.0001 | |
| C- | 318.36 | 1 | 318.36 | 18.45 | 0.0006 | |
| [NaBH₃CN] | | | | | | |
| Residual | 276.05 | 16 | 17.25 | | | |
| Lack of Fit | 248.85 | 11 | 22.62 | 4.16 | 0.0638 | not |
| | | | | | | significant |
| Pure Error | 27.20 | 5 | 5.44 | | | |
| Cor Total | 1916.11 | 19 | | | | |

| | | | |
|---|---|---|---|
| Std. Dev. | 4.15 | R-Squared | 0.8559 |
| Mean | 24.83 | Adj R-Squared | 0.8289 |
| C.V. % | 16.73 | Pred R-Squared | 0.7342 |
| PRESS | 509.31 | Adeq Precision | 21.521 |
| −2 Log Likelihood | 109.25 | BIC | 121.24 |
| | | AICc | 119.92 |

| | Coefficient | | Standard | 95% CI | 95% CI | |
|---|---|---|---|---|---|---|
| Factor | Estimate | df | Error | Low | High | VIF |
| Intercept | 24.83 | 1 | 0.93 | 22.86 | 26.80 | |
| A-[GAC₁₉₇] | −3.35 | 1 | 1.31 | −6.13 | −0.56 | 1.00 |
| B-[CRM] | 11.00 | 1 | 1.31 | 8.21 | 13.78 | 1.00 |
| C- | −5.64 | 1 | 1.31 | −8.43 | −2.86 | 1.00 |
| [NaBH₃CN] | | | | | | |

### Aspects of the Invention

1. A polysaccharide conjugate comprising or consisting of a one or more polysaccharide conjugated to a carrier polypeptide, wherein the carrier polypeptide is:
(a) selected from the group consisting of a Streptococcus pyogenes SpyAD (Spy0269, GAS40), a Streptococcus pyogenes SpyCEP (Spy0416, GAS57), or Streptococcus pyogenes SLO (Spy0167, GAS25);
(b) CRM₁₉₇; and
(c) a variant, fragment and/or fusion of (a) or (b).

2. The polysaccharide conjugate of aspect 1, wherein the carrier polypeptide is:
(a) a Streptococcus pyogenes SpyAD (Spy0269); or
(b) a variant, fragment and/or fusion of a Streptococcus pyogenes SpyAD (Spy0269).

3. The polysaccharide conjugate of aspect 1 or 2 wherein the Streptococcus pyogenes SpyAD (Spy0269) comprises or consists of:
(i) the amino acid sequence of SEQ ID NO: 1 or SEQ ID NO: 2;
(ii) an amino acid sequence comprising from 1 to 10 single amino acid alterations compared to SEQ ID NO: 1 or SEQ ID NO: 2;
(iii) an amino acid sequence with at least 70% sequence identity with SEQ ID NO: 1 or SEQ ID NO: 2; and/or
(iv) a fragment of at least 10 consecutive amino acids from SEQ ID NO: 1 or SEQ ID NO: 2, for example, at least 20, 30, 40, 50, 60, 70, 80, 90, 100, 125, 150, 175, 200, 250, 275, 280, 290, 300, 310, 320, 330, 340, or 350 consecutive amino acids from SEQ ID NO: 1 or SEQ ID NO: 2.

4. The polysaccharide conjugate of aspect 1, wherein the carrier polypeptide is:
(a) a Streptococcus pyogenes SpyCEP (Spy0416);
(b) a variant, fragment and/or fusion of a Streptococcus pyogenes SpyCEP (Spy0416).

5. The polysaccharide conjugate of aspect 4 wherein the Streptococcus pyogenes SpyCEP (Spy0416) comprises or consists of:
(i) the amino acid sequence of SEQ ID NO: 3 or SEQ ID NO: 4;
(ii) an amino acid sequence comprising from 1 to 10 single amino acid alterations compared to SEQ ID NO: 3 or SEQ ID NO: 4;
(iii) an amino acid sequence with at least 70% sequence identity with SEQ ID NO: 3 or SEQ ID NO: 4; and/or
(iv) a fragment of at least 10 consecutive amino acids from SEQ ID NO: 3 or SEQ ID NO: 4, for example, at least 20, 30, 40, 50, 60, 70, 80, 90, 100, 125, 150, 175, 200, 250, 275, 280, 290, 300, 310, 320, 330, 340, 350, 500, 750, 1000, 1250, 1500, 1550, 1600, 1610, 1620, 1630, 1640, 1650 or 1660 consecutive amino acids from SEQ ID NO: 3 or SEQ ID NO:4.

6. The polysaccharide conjugate of aspect 1, wherein the carrier polypeptide is:
(a) a Streptococcus pyogenes Slo (Spy0167); or
(b) a variant, fragment and/or fusion of a Streptococcus pyogenes Slo (Spy0167).

7. A polysaccharide conjugate 6 wherein the Streptococcus pyogenes Slo (Spy0167) comprises or consists of:
(i) the amino acid sequence of SEQ ID NO: 5 or SEQ ID NO: 6;
(ii) an amino acid sequence comprising from 1 to 10 single amino acid alterations compared to SEQ ID NO: 5 or SEQ ID NO: 6;
(iii) an amino acid sequence with at least 70% sequence identity with SEQ ID NO: 5 or SEQ ID NO: 6; and/or
(iv) a fragment of at least 10 consecutive amino acids from SEQ ID NO: 5 or SEQ ID NO: 6, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, 540, 550, 560 or 570 consecutive amino acids from SEQ ID NO: 5 or SEQ ID NO: 6.

8. The polysaccharide conjugate of aspect 1, wherein the carrier polypeptide is:
(a) CRM197; or
(b) a variant, fragment and/or fusion of CRM197.

9. A polysaccharide conjugate 8 wherein the CRM197 comprises or consists of:
(i) the amino acid sequence of SEQ ID NO: 7;
(ii) an amino acid sequence comprising from 1 to 10 single amino acid alterations compared to SEQ ID NO: 7;
(iii) an amino acid sequence with at least 70% sequence identity with SEQ ID NO: 7; and/or
(iv) a fragment of at least 10 consecutive amino acids from SEQ ID NO: 7, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, or 535 consecutive amino acids from SEQ ID NO: 6.

10. The polysaccharide conjugate of any one of the preceding aspects wherein the one or more polysaccharide is a microbial polysaccharide such as a bacterial polysaccharide, an archaea polysaccharide, a fungal polysaccharide, or a protist polysaccharide.

11. The polysaccharide conjugate of aspect 10 wherein the microbe is a pathogen, for example, a human pathogen.

12. The polysaccharide conjugate of any one of the preceding aspects wherein the one or more polysaccharide is surface-expressed.

13. The polysaccharide conjugate of any one of the preceding aspects wherein the one or more polysaccharide is a bacterial polysaccharide, for example, a polysaccharide of a bacterium selected from the group consisting of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

14. The polysaccharide conjugate of any one of the preceding aspects wherein the one or more polysaccharide comprises or consists of deoxy sugar monomers, for example, deoxy sugars selected from the group consisting of rhamnose (6-deoxy-L-mannose), fuculose (6-deoxy-L-tagatose), or fucose (6-deoxy-L-galactose).

15. The polysaccharide conjugate of any one of the preceding aspects wherein the one or more polysaccharide comprises side chain, for example, side chain comprises or consisting of N-acetylglucosamine (GlcNAc).

16. The polysaccharide conjugate of any one of the preceding aspects wherein an average of 1, 1.5 2, 2.5 3, 3.5 4, 4.5, 5, 6, 7, 8, 9 10, 11, 12, 13, 14, 15, 16, 17, 18, 19, or 20 polysaccharide molecules are conjugated to the carrier polypeptide.

17. The polysaccharide conjugate of any one of the preceding aspects wherein the one or more polysaccharide comprises or consists of:
I. a single molecular species; or
II. a mixture of molecular species, for example, 2, 3, 4, 5 6, 7, 8, 9, 10, 11, 12, 13, 14, 15, 16, 17, 18, 19, or 20 molecular species.

18. The polysaccharide conjugate of any one of the preceding aspects wherein the one or more polysaccharide is conjugated to the carrier protein directly.

19. The polysaccharide conjugate of any one of the preceding aspects wherein the one or more polysaccharide is conjugated to the carrier protein via a linker.

20. The polysaccharide conjugate of any one of the preceding aspects wherein the one or more polysaccharide has a molecular weight of less than 100 kDa (e.g. less than 80, 70, 60, 50, 40, 30, 25, 20, 15, 14, 13, 12, 11, 10, 9, 8, 7, 6, 5, 4, 3, 2 or 1 kDa).

21. The polysaccharide conjugate of any one of the preceding aspects wherein the one or more polysaccharide has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or fewer monosaccharide units.

22. The polysaccharide conjugate of any one of the preceding aspects wherein the one or more polysaccharide comprises or consists of a capsular polysaccharide of a bacterium selected from the group consisting of: Haemophilus influenzae type B and type A; Neisseria meningitidis serogroups A, C, W135, X and Y; Streptococcus pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F; Salmonella including Salmonella enterica serovar Typhi Vi, either full length or fragmented (indicated as fyi); Shigella sp, group A and B Streptococcus (GAS and GBS respectively).

23. The polysaccharide conjugate of any one of the preceding aspects wherein the one or more polysaccharide is conjugated to the carrier protein (a) by an amine formed from the reducing end residue from an aldehyde or ketone group from the terminal residue of the polysaccharide chain of the polysaccharide chain, and a lysine of the carrier protein; and/or (b) by one or more aldehyde groups formed from oxidised backbone and/or side chains of the polysaccharide (for example, for GAC, vicinal diols (1,2-diols) of the GlcNAc side chain) and a lysine of the carrier protein.

24. The polysaccharide conjugate of any one of the preceding aspects further comprising an adjuvant, for example, aluminum hydroxide, Alhydrogel (aluminum hydroxide 2% wet gel suspension, Croda International Plc), and Alum-TLR7.

25. The polysaccharide conjugate of any one of the preceding aspects wherein:
I. the carrier polypeptide comprises or consists of the amino acid sequence according to SEQ ID NO: 1; and
II. the one or more polysaccharide conjugated to a carrier polypeptide comprises or consists of GAC (group A carbohydrate of Streptococcus pyogenes).

26. The polysaccharide conjugate of any one of the preceding aspects wherein:
I. the carrier polypeptide comprises or consists of the amino acid sequence according to SEQ ID NO: 3 (mutant SpyCEP); and
II. the one or more polysaccharide conjugated to a carrier polypeptide comprises or consists of GAC (group A carbohydrate of Streptococcus pyogenes).

27. The polysaccharide conjugate of any one of the preceding aspects wherein:
I. the carrier polypeptide comprises or consists of the amino acid sequence according to SEQ ID NO: 5 (SLO); and
II. the one or more polysaccharide conjugated to a carrier polypeptide comprises or consists of GAC (group A carbohydrate of Streptococcus pyogenes).

28. The polysaccharide conjugate of any one of the preceding aspects wherein:
I. the carrier polypeptide comprises or consists of the amino acid sequence according to SEQ ID NO: 7 (CRM197); and
II. the one or more polysaccharide conjugated to a carrier polypeptide comprises or consists of GAC (group A carbohydrate of Streptococcus pyogenes).

29. A vaccine comprising the polysaccharide conjugate of any one of aspects 1-28.
30. The vaccine of aspect 29 further comprising an adjuvant.

31. The vaccine of aspect 29 or aspect 30 further comprising one or more additional antigen, for example, a bacterial antigen selected from the group consisting of antigens of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

32. A polysaccharide conjugate of any one of aspects 1-28 or a vaccine of any one of aspects 29-31 for use in medicine.

33. A polysaccharide conjugate of any one of aspects 1-28 or a vaccine of any one of aspects 29-31 for use in raising an immune response in a mammal, for example, for treating and/or preventing one or more disease.

34. Use of a polysaccharide conjugate of any one of aspects 1-28 or a vaccine of any one of aspects 29-31 for raising an immune response in a mammal, for example, for treating and/or preventing one or more disease.

35. Use of a polysaccharide conjugate of any one of aspects 2-28 or a vaccine of any one of aspects 29-31 for the manufacture of a medicament for raising an immune response in a mammal, for example, for treating and/or preventing one or more disease.

36. A method of raising an immune response in a mammal, the method comprising or consisting of administering the mammal with an effective amount of a polysaccharide conjugate of any one of aspects 2-18 or a vaccine of any one of aspects 29-31.

37. A method of oxidising polysaccharide comprising the steps of:
I. oxidisation of polysaccharide by reacting:
   i. polysaccharide, for example, at a concentration of 0.1-100 mg/ml, e.g., 0.5-50, 0.5-25, 1-10, 2.5-7.5, 4-6 or 5 mg/mL, with
   ii. oxidising agent (for example, NaIO₄ [sodium periodate+, KMnO₄ [potassium permanganate], periodic acid [HIO₄], or lead tetra-acetate [Pb(OAc)₄]), at a concentration 0.5-10M,
   iii. in a suitable buffer (for example, 200 mM phosphate buffer, or borate buffer) pH 3-9, for example, pH 5-8 (for example, pH5 or pH 8),
   iv. at a suitable temperature (for example, 20-30° C., such as 25° C.),
   v. for a suitable time (for example, 15 min-5 hr, such as, 30 min-3 hr, 30 min-1 hr, or 30 mins);
II. (optionally) quenching of residual NaIO₄ by:
   i. addition of a suitable amount of reducing agent, for example, Na₂SO₃ (sodium sulfite), for example, at a molar excess with respect to the concentration of NaIO₄ in step I(ii), for example, 5-10 times the concentration of NaIO₄ in step I(ii), or 16 mM,
   ii. at a suitable temperature (e.g., 20-30° C., room temperature, or 25° C.),
   iii. for a suitable time (e.g., 10-30 min, or 15 min);
III. (optionally) purification and/or concentration of oxidised polysaccharide, for example, using a method selected from the group consisting of lyophilisation, centrifugal evaporation, rotary evaporation, and tangential flow filtration.

38. A method of conjugating oxidised polysaccharide comprising the steps of:
A. reacting:
   a. oxidised polysaccharide (e.g., oxidised polysaccharide of aspect 37) at a concentration of 5-75 mg/mL (for example, 40 mg/mL) with;
   b. protein at a concentration of 5-75 mg/mL (for example 40 mg/mL); and
   c. NaBH₃CN (sodium cyanoborohydride) concentration of 0.5-10.0 mg/ml;
   d. In borate buffer pH 7-9, for example, pH 7.5-8.5, pH8;
   e. at a suitable temperature (for example, 17.5-42.5° C., room temperature, 25° C., 30° C. or 37° C.),
   f. for a suitable time (e.g., 1 hr, 2 hr, 4 hr, 6 hr, 0.5 to 3 days, 1 day or 2 days;
B. (optionally) quenching of residual aldehydes of oxidised polysaccharide by:
   a. addition of a suitable amount of NaBH₄ (e.g., an NaBH₄:polysaccharide ratio [w/w] of 0.5:1, or, for example, at a molar excess with respect to the aldehyde groups generated or moles of oxidized polysaccharide, for example, 5-10 times, 50 times, 100 times or 1000 times),
   b. at a suitable temperature (e.g., 20-30° C., 25° C., or room temperature),
   c. for a suitable time (e.g., 1 to 12 hr, 2-4 hr).
C. (optionally) purification of the polysaccharide conjugate resulting from step (B) by tangential flow filtration (TFF) and/or sterile filtration (e.g., TFF followed by sterile filtration).

39. A method of conjugating polysaccharide to polypeptide comprising or consisting of steps (1) to (IV) of aspect 37 and steps (A) to (C) of aspect 38.

40. The method of any one of aspects 37-39 wherein the polysaccharide is a polysaccharide described in any one of aspects 1-28, for example, GAC.

41. The method of any one of aspects 37-40 wherein the protein is a protein described in any one of aspects 1-28, for example, SpyAD (e.g., SEQ ID NO: 1 or SEQ ID NO: 2), SpyCEP (e.g., SEQ ID NO: 3 or SEQ ID NO: 4), Slo (e.g., SEQ ID NO: 5 or SEQ ID NO: 6) or CRM197 (e.g., SEQ ID NO: 7).

42. The method of any one of aspects 37-41 wherein the method product is a polysaccharide conjugate described in any one of aspects 1-28, for example:
I. SpyAD (e.g., SEQ ID NO: 1 or SEQ ID NO: 2) conjugated to GAC;
II. SpyCEP (e.g., SEQ ID NO: 3 or SEQ ID NO: 4) conjugated to GAC;
III. Slo (e.g., SEQ ID NO: 5 or SEQ ID NO: 6) conjugated to GAC; or
IV. CRM197 (e.g., SEQ ID NO: 7) conjugated to GAC.

43. The method according to any one of aspects 38-42 wherein reactions are performed below the Tm of the polypeptide, for example, at least 0.5, 1.0, 1.5, 2.0, 2.5, 3.0, 4.0, 5.0, 6.0, 7.0 or 7.5° C. below the Tm of the polypeptide.

44. A polysaccharide conjugate produced according to the method of any one of aspects 37-42.

45. A polysaccharide conjugate, use, or method as described anywhere in the specification and/or figures herein.

### Further Aspects of the Invention

1. A polysaccharide conjugate comprising or consisting of one or more polysaccharide conjugated to a carrier polypeptide, wherein the carrier polypeptide comprises a polypeptide:
(a) selected from the group consisting of a Streptococcus pyogenes SpyAD, a Streptococcus pyogenes SpyCEP, and a Streptococcus pyogenes SLO; or
(b) CRM₁₉₇; or
(c) a variant, fragment and/or fusion of (a) or (b).

2. The polysaccharide conjugate of aspect 1, wherein the carrier polypeptide is:
(a) a Streptococcus pyogenes SpyAD (Spy0269); or
(b) a variant, fragment and/or fusion of a Streptococcus pyogenes SpyAD (Spy0269).

3. The polysaccharide conjugate of aspect 1 or 2 wherein the carrier polypeptide comprises or consists of:
(i) an amino acid sequence of SEQ ID NO: 1 or SEQ ID NO: 2;
(ii) an amino acid sequence that varies from SEQ ID NO: 1 or SEQ ID NO: 2 by from 1 to single amino acid alterations;
(iii) an amino acid sequence with at least 70%, 80%, 85%, 90%, 95%, 96%, 97%, 98%, 99% or at least 99.5% sequence identity with SEQ ID NO: 1 or SEQ ID NO: 2; and/or
(iv) a fragment of at least 10 consecutive amino acids from SEQ ID NO: 1 or SEQ ID NO: 2, for example, at least 20, 30, 40, 50, 60, 70, 80, 90, 100, 125, 150, 175, 200, 250, 275, 280, 290, 300, 310, 320, 330, 340, or 350 consecutive amino acids from SEQ ID NO: 1 or SEQ ID NO: 2.

4. The polysaccharide conjugate of aspect 3, wherein the carrier polypeptide comprises or consists of an amino acid having at least 95% identity with a fragment of at least 300 amino acids of SEQ ID NO: 1 or SEQ ID NO: 2.

5. The polysaccharide conjugate of aspect 3 or aspect 4, wherein the carrier polypeptide comprises or consist of an amino acid having at least 95% identity with SEQ ID NO: 1 or SEQ ID NO: 2.

6. The polysaccharide conjugate of any one of the preceding aspects, wherein the carrier polypeptide is:
(a) a Streptococcus pyogenes SpyCEP (Spy0416); or
(b) a variant, fragment and/or fusion of a Streptococcus pyogenes SpyCEP (Spy0416).

7. The polysaccharide conjugate of aspect 6, wherein the carrier polypeptide comprises or consists of:
(i) an amino acid sequence of SEQ ID NO: 3 or SEQ ID NO: 4;
(ii) an amino acid sequence that varies from SEQ ID NO: 3 or SEQ ID NO: 4 by from 1 to single amino acid alterations;
(iii) an amino acid sequence with at least 70%, 80%, 85%, 90%, 95%, 96%, 97%, 98%, 99% or at least 99.5% sequence identity with SEQ ID NO: 3 or SEQ ID NO: 4; and/or
(iv) a fragment of at least 10 consecutive amino acids from SEQ ID NO: 3 or SEQ ID NO: 4, for example, at least 20, 30, 40, 50, 60, 70, 80, 90, 100, 125, 150, 175, 200, 250, 275, 280, 290, 300, 310, 320, 330, 340, 350, 500, 750, 1000, 1250, 1500, 1550, 1600, 1610, 1620, 1630, 1640, 1650 or 1660 consecutive amino acids from SEQ ID NO: 3 or SEQ ID NO: 4.

8. The polysaccharide conjugate of aspect 7, wherein the carrier polypeptide comprises or consists of an amino acid having at least 95% identity with a fragment of at least 1500 amino acids of SEQ ID NO: 3 or SEQ ID NO: 4.

9. The polysaccharide conjugate of aspect 7 or aspect 8, wherein the carrier polypeptide comprises or consist of an amino acid having at least 95% identity with SEQ ID NO: 3 or SEQ ID NO: 4.

10. The polysaccharide conjugate of any one of the preceding aspects, wherein the carrier polypeptide comprises:
(a) a Streptococcus pyogenes Slo (Spy0167); or
(b) a variant, fragment and/or fusion of a Streptococcus pyogenes Slo (Spy0167).

11. The polysaccharide conjugate of aspect 10, wherein the carrier polypeptide comprises or consists of:
(i) an amino acid sequence of SEQ ID NO: 5 or SEQ ID NO: 6;
(ii) an amino acid sequence that varies from SEQ ID NO: 5 or SEQ ID NO: 6 by from 1 to single amino acid alterations;
(iii) an amino acid sequence with at least 70%, 80%, 85%, 90%, 95%, 96%, 97%, 98%, 99% or at least 99.5% sequence identity with SEQ ID NO: 5 or SEQ ID NO: 6; and/or
(iv) a fragment of at least 10 consecutive amino acids from SEQ ID NO: 5 or SEQ ID NO: 6, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, 540, 550, 560 or 570 consecutive amino acids from SEQ ID NO: 5 or SEQ ID NO: 6.

12. The polysaccharide conjugate of aspect 11, wherein the carrier polypeptide comprises or consists of an amino acid having at least 95% identity with a fragment of at least 500 amino acids of SEQ ID NO: 5 or SEQ ID NO: 6.

13. The polysaccharide conjugate of aspect 11 or aspect 12, wherein the carrier polypeptide comprises or consist of an amino acid having at least 95% identity with SEQ ID NO: 5 or SEQ ID NO: 6.

14. The polysaccharide conjugate of aspect 1, wherein the carrier polypeptide is:
(a) CRM197; or
(b) a variant, fragment and/or fusion of CRM197.

15. The polysaccharide conjugate of aspect 14, wherein the CRM197 comprises or consists of:
(i) a polypeptide having an amino acid sequence of SEQ ID NO: 7;
(ii) an amino acid sequence that varies from SEQ ID NO: 7 by from 1 to 10 single amino acid alterations;
(iii) an amino acid sequence with at least 70%, 80%, 85%, 90%, 95%, 96%, 97%, 98%, 99% or at least 99.5% sequence identity with SEQ ID NO: 7; and/or
(iv) a fragment of at least 10 consecutive amino acids from SEQ ID NO: 7, for example, at least 20, 30, 40, 50, 60, 70, 80, 90, 100, 125, 150, 175, 200, 250, 275, 280, 290, 300, 310, 320, 330, 340, 350, 400, 450, 500, 510, 520, 530, or 535 consecutive amino acids from SEQ ID NO: 7.

16. The polysaccharide conjugate of aspect 15, wherein the carrier polypeptide comprises or consists of an amino acid having at least 95% identity with a fragment of at least 500 amino acids of SEQ ID NO: 7.

17. The polysaccharide conjugate of aspect 15 or aspect 16, wherein the carrier polypeptide comprises or consist of an amino acid having at least 95% identity with SEQ ID NO: 7.

18. The polysaccharide conjugate of any one of the preceding aspects, wherein the one or more polysaccharide is a microbial polysaccharide such as a bacterial polysaccharide, an archaeal polysaccharide, a fungal polysaccharide, or a protist polysaccharide.

19. The polysaccharide conjugate of aspect 18, wherein the microbe is a pathogen, for example, a human pathogen.

20. The polysaccharide conjugate of any one of the preceding aspects, wherein the one or more polysaccharide is surface-expressed.

21. The polysaccharide conjugate of any one of the preceding aspects wherein the one or more polysaccharide is a bacterial polysaccharide, for example, a polysaccharide of a bacterium selected from the group consisting of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

22. The polysaccharide conjugate of any one of the preceding aspects, wherein the one or more polysaccharide comprises or consists of deoxy sugar monomers, for example, deoxy sugars selected from the group consisting of rhamnose (6-deoxy-L-mannose), fuculose (6-deoxy-L-tagatose), and fucose (6-deoxy-L-galactose).

23. The polysaccharide conjugate of any one of the preceding aspects, wherein the one or more polysaccharide comprises a side chain, for example, a side chain comprising or consisting of N-acetylglucosamine (GlcNAc).

24. The polysaccharide conjugate of any one of the preceding aspects wherein an average of at least 1, 1.5 2, 2.5 3, 3.5 4, 4.5, 5, 6, 7, 8, 9 10, 11, 12, 13, 14, 15, 16, 17, 18, 19, or 20 polysaccharide molecules are conjugated to the carrier polypeptide.

25. The polysaccharide conjugate of any one of the preceding aspects, wherein the ratio of polysaccharide to carrier polypeptide is greater than 0.3, greater than 0.4, between 0.3 and 1.0, or between 0.4 and 0.6 (w/w).

26. The polysaccharide conjugate of any one of the preceding aspects wherein the one or more polysaccharide comprises or consists of:
I. a single molecular species; or
II. a mixture of molecular species, for example, at least 2, 3, 4, 5, 6, 7, 8, 9, 10, 11, 12, 13, 14, 15, 16, 17, 18, 19, or 20 molecular species.

27. The polysaccharide conjugate of any one of the preceding aspects wherein the one or more polysaccharide is conjugated to the carrier protein directly.

28. The polysaccharide conjugate of any one of the preceding aspects wherein the one or more polysaccharide is conjugated to the carrier protein via a linker.

29. The polysaccharide conjugate of any one of the preceding aspects wherein the one or more polysaccharide has a molecular weight of less than 100 kDa (e.g. less than 80, 70, 60, 50, 40, 30, 25, 20, 15, 14, 13, 12, 11, 10, 9, 8, 7, 6, 5, 4, 3, 2 or 1 kDa).

30. The polysaccharide conjugate of any one of the preceding aspects wherein the one or more polysaccharide has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or fewer monosaccharide units.

31. The polysaccharide conjugate of any one of the preceding aspects, wherein the one or more polysaccharide comprises or consists of a capsular polysaccharide of a bacterium selected from the group consisting of: Haemophilus influenzae type B and type A; Neisseria meningitidis serogroups A, C, W135, X and Y; Streptococcus pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F; Salmonella including Salmonella enterica serovar Typhi Vi, either full length or fragmented (indicated as fyi); Shigella sp, group A and B Streptococcus (GAS and GBS respectively).

32. The polysaccharide conjugate of any one of the preceding aspects, wherein the one or more polysaccharide comprises Group A Carbohydrate (GAC).

33. The polysaccharide conjugate of any one of the preceding aspects wherein the one or more polysaccharide is conjugated to the carrier protein (a) by an amine formed from the reducing end residue from an aldehyde or ketone group from the terminal residue of the polysaccharide chain of the polysaccharide chain, and a lysine of the carrier protein; and/or (b) by one or more aldehyde groups formed from oxidised backbone and/or side chains of the polysaccharide (for example, for GAC, vicinal diols (1,2-diols) of the GlcNAc side chain) and a lysine of the carrier protein.

34. The polysaccharide conjugate of any one of the preceding aspects wherein:
I. the carrier polypeptide comprises or consists of:
   the amino acid sequence according to SEQ ID NO: 1; and
II. the one or more polysaccharide conjugated to a carrier polypeptide comprises or consists of GAC (group A carbohydrate of Streptococcus pyogenes).

35. The polysaccharide conjugate of any one of the preceding aspects wherein:
I. the carrier polypeptide comprises or consists of:
   the amino acid sequence according to SEQ ID NO: 3 (mutant SpyCEP); and
II. the one or more polysaccharide conjugated to a carrier polypeptide comprises or consists of GAC (group A carbohydrate of Streptococcus pyogenes).

36. The polysaccharide conjugate of any one of the preceding aspects wherein:
I. the carrier polypeptide comprises or consists of:
   the amino acid sequence according to SEQ ID NO: 5 (SLO); and
II. the one or more polysaccharide conjugated to a carrier polypeptide comprises or consists of GAC (group A carbohydrate of Streptococcus pyogenes).

37. The polysaccharide conjugate of any one of the preceding aspects wherein:
I. the carrier polypeptide comprises or consists of:
   the amino acid sequence according to SEQ ID NO: 7 (CRM197); and
II. the one or more polysaccharide conjugated to a carrier polypeptide comprises or consists of GAC (group A carbohydrate of Streptococcus pyogenes).

38. A composition comprising the polysaccharide conjugate of any one of aspects 1-37, the composition further comprising an adjuvant, for example, aluminium hydroxide, Alhydrogel (aluminium hydroxide 2% wet gel suspension, Croda International Plc), or Alum-TLR7.

39. An immunogenic composition comprising the polysaccharide conjugate of any one of aspects 1-37.

40. A vaccine comprising the polysaccharide conjugate of any one of aspects 1-37.

41. The vaccine of aspect 40 further comprising an adjuvant, for example, aluminium hydroxide, Alhydrogel (aluminium hydroxide 2% wet gel suspension, Croda International Plc), or Alum-TLR7.

42. The composition of aspect 38, the immunogenic composition of aspect 39, or the vaccine of aspect 40 or aspect 41, further comprising one or more additional antigen, for example, a bacterial antigen selected from the group consisting of antigens of: Actinomyces (e.g., A. israelii), Bacillus (e.g., B. anthracis or B. cereus), Bartonella (e.g., B. henselae, or B. quintana), Bordetella (e.g., B. pertusis), Borrelia (e.g., B. burgdorferi, B. Borrelia garinii, B. afzelii, B. recurrentis), Brucella (e.g., B. abortus, B. canis, B. melitensis, or B. suis), Campylobacter (e.g., C. jejuni), Chlamydia (e.g., C. pneumoniae or C. trachomatis), Chlamydophila (e.g., C. psittaci), Clostridium (e.g., C. botulinum, C. difficile, C. perfringens, C. tetani), Corynebacterium (e.g., C. diphtheriae), Enterococcus (e.g., E. faecalis, or E. faecium), Escherichia (e.g., E. coli), Francisella (e.g., F. tularensis), Haemophilus (e.g., H. influenzae), Helicobacter (e.g., H. pylori), Klebsiella (e.g., K. pneumoniae and K. oxytoca), Legionella (e.g., L. pneumophila), Leptospira (e.g., L. interrogans, L. santarosai, L. weilii, L. noguchii), Listeria (e.g., L. monocytogenes), Mycobacterium (e.g., M. leprae, M. tuberculosis, or M. ulcerans), Mycoplasma (e.g., M. pneumoniae), Neisseria (e.g., N. gonorrhoeae or N. meningitidis), Pseudomonas (e.g., P. aeruginosa), Rickettsia (e.g., R. rickettsii), Salmonella (e.g., S. typhi, S. enteritidis, S. paratyphi, S. typhimurium, or S. choleraesuis), Shigella (e.g., S. boydii, S. flexneri, S. sonnei, or S. dysenteriae), Staphylococcus (e.g., S. aureus, S. epidermis, or S. saprophyticus), Streptococcus (e.g., S. agalactiae, S. pneumoniae, or S. pyogenes), Treponema (e.g., T. pallidum), Ureaplasma (e.g., U. urealyticum), Vibrio (e.g., V. cholerae), or Yersinia (e.g., Y. pestis, Y. enterocolitica, or Y. pseudotuberculosis).

43. A polysaccharide conjugate of any one of aspects 1-37, the composition of aspect 38, the immunogenic composition of aspect 39, or a vaccine of any one of aspects 40-42 for use in medicine.

44. A polysaccharide conjugate of any one of aspects 1-37, the composition of aspect 38, the immunogenic composition of aspect 39, or a vaccine of any one of aspects 40-42 for use in raising an immune response in a mammal, for example, for treating and/or preventing one or more disease.

45. A polysaccharide conjugate of any one of aspects 1-37, the composition of aspect 38, the immunogenic composition of aspect 39, or a vaccine of any one of aspects 40-42 for use in treating and/or preventing GAS infection.

46. Use of a polysaccharide conjugate of any one of aspects 1-37, the composition of aspect 38, the immunogenic composition of aspect 39, or a vaccine of any one of aspects 40-42 for raising an immune response in a mammal, for example, for treating and/or preventing one or more disease.

47. Use of a polysaccharide conjugate of any one of aspects 1-37, the composition of aspect 38, the immunogenic composition of aspect 39, or a vaccine of any one of aspects 29-31 for treating and/or preventing GAS infection.

48. Use of a polysaccharide conjugate of any one of aspects 1-37, the composition of aspect 38, the immunogenic composition of aspect 39, or a vaccine of any one of aspects 40-42 for the manufacture of a medicament for raising an immune response in a mammal, for example, for treating and/or preventing one or more disease.

49. Use of a polysaccharide conjugate of any one of aspects 1-37, the composition of aspect 38, the immunogenic composition of aspect 39, or a vaccine of any one of aspects 40-42 for the manufacture of a medicament for treating and/or preventing GAS infection.

50. A method of raising an immune response in a mammal, the method comprising or consisting of administering the mammal with an effective amount of a polysaccharide conjugate of any one of aspects 1-37, the composition of aspect 38, the immunogenic composition of aspect 39 or a vaccine of any one of aspects 40-42.

51. A method of oxidising polysaccharide comprising the steps of:
I. oxidisation of polysaccharide by reacting:
   i. polysaccharide, for example, at a concentration of 0.1-100 mg/ml, e.g., 0.5-50, 0.5-25, 1-10, 2.5-7.5, 4-6 or 5 mg/mL, with
   ii. oxidising agent (for example, NaIO₄ [sodium periodate+, KMnO₄ [potassium permanganate], periodic acid [HIO₄], or lead tetra-acetate [Pb(OAc)₄]), at a concentration 0.5-10M,
   iii. in a suitable buffer (for example, 200 mM phosphate buffer, or borate buffer) pH 3-9, for example, pH 5-8 (for example, pH5 or pH 8),
   iv. at a suitable temperature (for example, 20-30° C., such as 25° C.),
   v. for a suitable time (for example, 15 min-5 hr, such as, 30 min-3 hr, 30 min-1 hr, or 30 mins);
II. (optionally) quenching of residual NaIO₄ by:
   i. addition of a suitable amount of reducing agent, for example, Na₂SO₃ (sodium sulfite), for example, at a molar excess with respect to the concentration of NaIO₄ in step I(ii), for example, 5-10 times the concentration of NaIO₄ in step I(ii), or 16 mM,
   ii. at a suitable temperature (e.g., 20-30° C., room temperature, or 25° C.),
   iii. for a suitable time (e.g., 10-30 min, or 15 min);
III. (optionally) purification and/or concentration of oxidised polysaccharide, for example, using a method selected from the group consisting of lyophilisation, centrifugal evaporation, rotary evaporation, and tangential flow filtration.

52. A method of conjugating oxidised polysaccharide comprising the steps of:
A. reacting:
   a. oxidised polysaccharide (e.g., oxidised polysaccharide of aspect 37) at a concentration of 5-75 mg/mL (for example, 40 mg/mL) with;
   b. protein at a concentration of 5-75 mg/mL (for example 40 mg/mL); and
   c. NaBH₃CN (sodium cyanoborohydride) concentration of 0.5-10.0 mg/ml;
   d. In borate buffer pH 7-9, for example, pH 7.5-8.5, pH8;
   e. at a suitable temperature (for example, 17.5-42.5° C., room temperature, 25° C., 30° C. or 37° C.),
   f. for a suitable time (e.g., 1 hr, 2 hr, 4 hr, 6 hr, 0.5 to 3 days, 1 day or 2 days;
B. (optionally) quenching of residual aldehydes of oxidised polysaccharide by:
   g. addition of a suitable amount of NaBH₄ (e.g., an NaBH₄:polysaccharide ratio [w/w] of 0.5:1, or, for example, at a molar excess with respect to the aldehyde groups generated or moles of oxidized polysaccharide, for example, 5-10 times, 50 times, 100 times or 1000 times),
   h. at a suitable temperature (e.g., 20-30° C., 25° C., or room temperature),
   i. for a suitable time (e.g., 1 to 12 hr, 2-4 hr).
C. (optionally) purification of the polysaccharide conjugate resulting from step (B) by tangential flow filtration (TFF) and/or sterile filtration (e.g., TFF followed by sterile filtration).

53. A method of conjugating polysaccharide to polypeptide comprising or consisting of steps (I) to (III) of aspect 51 and steps (A) to (C) of aspect 52.

54. The method of any one of aspects 51-53 wherein the polysaccharide is a polysaccharide described in any one of aspects 1-37, for example, GAC.

55. The method of any one of aspects 51-54 wherein the protein is a protein described in any one of aspects 1-37, for example, SpyAD (e.g., SEQ ID NO: 1 or SEQ ID NO: 2), SpyCEP (e.g., SEQ ID NO: 3 or SEQ ID NO: 4), Slo (e.g., SEQ ID NO: 5 or SEQ ID NO: 6) or CRM197 (e.g., SEQ ID NO: 7).

56. The method of any one of aspects 51-55 wherein the method product is a polysaccharide conjugate described in any one of aspects 1-37, for example:
I. SpyAD (e.g., SEQ ID NO: 1 or SEQ ID NO: 2) conjugated to GAC;
II. SpyCEP (e.g., SEQ ID NO: 3 or SEQ ID NO: 4) conjugated to GAC;
III. Slo (e.g., SEQ ID NO: 5 or SEQ ID NO: 6) conjugated to GAC; or
IV. CRM197 (e.g., SEQ ID NO: 7) conjugated to GAC.

57. The method according to any one of aspects 52-56 wherein reactions are performed below the Tm of the polypeptide, for example, at least 0.5, 1.0, 1.5, 2.0, 2.5, 3.0, 4.0, 5.0, 6.0, 7.0 or 7.5° C. below the Tm of the polypeptide.

58. A polysaccharide conjugate produced according to the method of any one of aspects 51-56.

59. A polysaccharide conjugate, use, or method as described anywhere in the specification and/or figures herein.

60. A method of conjugating a GAC polysaccharide to a carrier protein comprising a step of oxidising the polysaccharide by reacting the polysaccharide with an oxidising agent.

61. The method of aspect 52-57 or 60, wherein the step of oxidising the polysaccharide by reacting the polysaccharide with an oxidising agent is performed in a suitable buffer, at a suitable temperature and for a suitable time.

62. A method of oxidising a polysaccharide comprising a step of oxidisation of the polysaccharide comprising the steps of:
I. oxidisation of the polysaccharide by reacting the polysaccharide with
   (a) an oxidising agent,
   (b) in a suitable buffer,
   (c) at a suitable temperature,
   (d) for a suitable time.

63. The method of aspect 51, 54 to 56, 61 or 62, wherein at least one of the polysaccharide concentration, the oxidising agent, the oxidising agent concentration, the suitable buffer, the suitable temperature and the suitable time used ensure that the method achieves at least 5%, at least 10%, at least 15%, between 10% and 30%, between 10% and 25%, or around 15% oxidation of the polysaccharide.

64. The method of aspect 63, wherein the polysaccharide concentration, the oxidising agent, the oxidising agent concentration, the suitable buffer, the suitable temperature and the suitable time used in the method ensures that the method achieves at least 5%, at least 10%, at least 15%, between 10% and 30%, between 10% and 25%, or around 15% oxidation of the polysaccharide.

65. The method of any one of aspects 51, 54 to 56, or 61 to 63, wherein the method is configured to achieve at least 5%, at least 10%, at least 15%, between 10% and 30%, between 10% and 25%, or around 15% oxidation of the polysaccharide.

66. The method of any one of aspects 51, 54 to 56, or 61 to 65, wherein the polysaccharide is GAC and at least one of the polysaccharide concentration, the oxidising agent, the oxidising agent concentration, the suitable buffer, the suitable temperature and the suitable time used in the method ensures that the method achieves a GAC recovery of at least 60%, at least 65%, at least 70%, at least 75%, between 60% and 100%, between 65% and 100%, between 70% and 90%, or between 75% and 90%.

67. The method of any one of aspects 51, 54 to 56, or 61 to 66, wherein the polysaccharide is GAC and the polysaccharide concentration, the oxidising agent, the oxidising agent concentration, the suitable buffer, the suitable temperature and the suitable time used in the method ensures that the method achieves a GAC recovery of at least 60%, at least 65%, at least 70%, at least 75%, between 60% and 100%, between 65% and 100%, between 70% and 90%, or between 75% and 90%.

68. The method of any one of aspects 51, 54 to 56, or 61 to 66, wherein the polysaccharide is GAC and the method is configured to achieve a GAC recovery of at least 60%, at least 65%, at least 70%, at least 75%, between 60% and 100%, between 65% and 100%, between 70% and 90%, or between 75% and 90%.

69. The method of any one of aspects 51, 54 to 56, or 61 to 68, wherein the polysaccharide concentration is 0.1-100 mg/ml, 0.5-50 mg/ml, 0.5-25 mg/ml, 1-10 mg/ml, 2.5-7.5 mg/ml, 4-6 mg/ml, or around 5 mg/ml.

70. The method of aspect 69, wherein the polysaccharide concentration is 1-10 mg/ml.

71. The method of any one of aspects 51, 54 to 56, or 61 to 70, wherein the oxidising agent is selected from the group consisting of sodium periodate (NaIO₄), potassium permanganate (KMnO₄), periodic acid (HlO₄), or lead tetra-acetate (Pb(OAc)₄).

72. The method of aspect 71, wherein the oxidising agent is NaIO₄.

73. The method of any one of aspects 51, 54 to 56, or 61 to 72, wherein the oxidising agent concentration is 0.1-25 mM, 0.5-10 mM, 1-10 mM, 2-10 mM, 5-10 mM, or around 8 mM.

74. The method of aspect 73, wherein the oxidising agent concentration is 2-10 mM or around 8 mM.

75. The method of any one of aspects 51, 54 to 56, or 61 to 74, wherein the step of oxidisation of the polysaccharide occurs in a reaction mixture comprising the polysaccharide, the oxidising agent and the suitable buffer, and the suitable buffer maintains the pH of the reaction mixture at pH 3-9, pH 5-9, pH 6-9, or around pH 8.

76. The method of aspect 75, wherein the suitable buffer maintains the pH of the reaction mixture at pH 5-9 or around pH 8.

77. The method of any one of aspects 51, 54 to 56, or 61 to 76, wherein the suitable buffer is phosphate buffer or borate buffer.

78. The method of any one of aspects 51, 54 to 56, or 61 to 77, wherein the suitable temperature is 20° C.-30° C., 22° C.-28° C., room temperature, or around 25° C.

79. The method of any one of aspects 51, 54 to 56, 61 to 78, wherein the suitable time is 15 min-5 hr, 30 min-3 hr, 30 min-1 hr, or around 30 min.

80. The method of any one of aspects 51, 54 to 56, 61 to 79, further comprising a step of quenching residual oxidising agent by addition of a suitable reducing agent.

81. The method of aspect 80, wherein the suitable reducing agent is sodium sulfite (Na₂SO₃).

82. The method of aspect 80 or 81, wherein the step of quenching residual oxidising agent is carried out at a temperature of 20° C. to 30° C., or around 25° C.

83. The method of any one of aspects 51, 54 to 56, or 60 to 82, wherein the step of reacting the polysaccharide with an oxidising agent provides an oxidised polysaccharide and further comprising a step of purification and/or concentration of the oxidised polysaccharide.

84. The method of aspect 83, wherein the step of purification and/or concentration of the oxidised polysaccharide is carried out using a method comprising lyophilisation, centrifugal evaporation, rotary evaporation and/or tangential flow filtration.

85. The method of conjugating a GAC polysaccharide to a carrier protein of any one of aspects 51, 54 to 56, or 60 to 84, wherein the step of reacting the polysaccharide with an oxidising agent provides an oxidised GAC polysaccharide and wherein the method further comprises a step of reacting the oxidised GAC polysaccharide with a carrier polypeptide.

86. The method of aspect 85, wherein the step of reacting the GAC oxidised polysaccharide with a carrier polypeptide comprises reacting the GAC oxidised polysaccharide with the carrier polypeptide and sodium cyanoborohydride in borate buffer, at a suitable temperature, for a suitable time.

87. The method of aspect 85 or 86, wherein the method does not comprise a purification step between the step of reacting the polysaccharide with an oxidising agent and the step of reacting the oxidised GAC polysaccharide with a carrier polypeptide.

88. A method of conjugating oxidised polysaccharide comprising a step of reacting:
a. oxidised polysaccharide with;
b. a carrier polypeptide/protein; and
c. sodium cyanoborohydride;
d. in borate buffer;
e. at a suitable temperature;
f. for a suitable time.

89. The method of any one of aspects 52 to 57, or 86 to 88, wherein at least one of the oxidised polysaccharide concentration, the carrier polypeptide/protein concentration, the sodium cyanoborohydride concentration, the pH of the borate buffer, and the suitable temperature used in the method ensures that the method achieves a polysaccharide to carrier polypeptide/protein ratio of at least 0.25, at least 0.3, at least 0.35, at least 0.4, between 0.25 and 1, between 0.3 and 0.8, or between 0.4 and 0.8.

90. The method of any one of aspects 52 to 57, or 86 to 89, wherein the oxidised polysaccharide concentration, the carrier polypeptide/protein concentration, the sodium cyanoborohydride concentration, the pH of the borate buffer, and the suitable temperature used in the method ensures that the method achieves a polysaccharide to carrier polypeptide/protein ratio of at least 0.25, at least 0.3, at least 0.35, at least 0.4, between 0.25 and 1, between 0.3 and 0.8, or between 0.4 and 0.8.

91. The method of any one of aspects 52 to 57, or 86 to 90, wherein the method is configured to achieve a polysaccharide to carrier polypeptide/protein ratio of at least 0.25, at least 0.3, at least 0.35, at least 0.4, between 0.25 and 1, between 0.3 and 0.8, or between 0.4 and 0.8.

92. The method any one of aspects 52 to 57, or 86 to 91, wherein the polysaccharide is GAC and at least one of the oxidised polysaccharide concentration, the carrier polypeptide/protein concentration, the sodium cyanoborohydride concentration, the pH of the borate buffer, and the suitable temperature used in the method ensures that the method achieves a GAC recovery of at least 25%, at least 30%, at least 35%, between 25% and 80%, between 30% and 70%, or between 35% and 60%.

93. The method of any one of aspects 52 to 57, or 86 to 92, wherein the polysaccharide is GAC and the oxidised polysaccharide concentration, the carrier polypeptide/protein concentration, the sodium cyanoborohydride concentration, the pH of the borate buffer, and the suitable temperature used in the method ensures that the method achieves a GAC recovery of at least 25%, at least 30%, at least 35%, between 25% and 80%, between 30% and 70%, or between 35% and 60%.

94. The method of any one of aspects 52 to 57, or 86 to 93, wherein the polysaccharide is GAC and the method is configured to achieve a GAC recovery of at least 25%, at least 30%, at least 35%, between 25% and 80%, between 30% and 70%, or between 35% and 60%.

95. The method of any one of aspects 52 to 57, or 86 to 94, wherein the ratio of polysaccharide to carrier polypeptide/protein to sodium cyanoborohydride is 1-20:1-20:1 mg/ml, 5-15:5-15:1 mg/ml, or around 8:8:1 w/w/v.

96. The method of any one of aspects 52 to 57, or 86 to 95, wherein the oxidised polysaccharide is at a concentration of 5-75 mg/ml, 10-50 mg/ml 20-60 mg/ml, or around 40 mg/ml.

97. The method of any one of aspects 52 to 57, or 86 to 96, wherein the carrier polypeptide/protein is at a concentration of 5-75 mg/ml, 10-50 mg/ml, 20-60 mg/ml, or around 40 mg/ml.

98. The method of any one of aspects 52 to 57, or 86 to 97, wherein the sodium cyanoborohydride is at a concentration of 0.5-10 mg/ml, 2-8 mg/ml, or around 5 mg/ml.

99. The method of any one of aspects 52 to 57, or 86 to 98, wherein the borate buffer is at a pH of 7-9, 7.8-8.5 or around 8.

100. The method of any one of aspects 52 to 57, or 86 to 99, wherein the suitable temperature is a temperature of 17.5-42.5° C., 20-40° C., around 25° C., around 28° C., around 30° C., or around 37° C.

101. The method of any one of aspects 52 to 57, or 86 to 100, wherein the suitable time is at least 1 hour, at least 5 hours, at least 24 hours, between 1 hour and 5 days, between 5 hours and 3 days, or around 2 days.

102. The method of any one of aspects 52 to 57 or 86 to 101 further comprising a step of quenching residual aldehydes on the oxidised polysaccharide by addition of a suitable amount of sodium borohydride (NaBH₄).

103. The method of any one of aspects 52 to 57 or 102, wherein the step of quenching residual aldehydes on the oxidised polysaccharide is carried out:
(i) by addition of NaBH₄ at an amount equivalent to an NaBH₄: polysaccharide ratio (w/w) of at least 0.1:1, at least 0.2:1, around 0.5:1, or with a molar excess of NaBH₄ with respect to the number of aldehyde groups generated or moles of oxidised polysaccharide of 5-1000 times, 10-500 times, 50-250 times, around 50 times, around 100 times or around 1000 times; and/or
(ii) at a temperature of 20-30° C., 22-28° C., around 25° C., or at room temperature; and/or
(iii) for a time of 1-12 hours or 2-4 hours.

104. The method of any one of aspects 52 to 57, or 85 to 105, wherein the step of reacting the oxidised polysaccharide or the oxidised GAC polysaccharide with a carrier polypeptide/protein provides a polysaccharide conjugate and the method further comprises a step of purification of the polysaccharide conjugate.

105. The method of any one of aspects 52 to 57 or 104, wherein the step of purification of the polysaccharide conjugate comprising tangential flow filtration (TFF) and/or sterile filtration.

106. A method of conjugating polysaccharide to carrier polypeptide/protein comprising the method of any one of aspects 51 or 60 to 84 followed by the method of any one of aspects 52 to 57 or 88 to 105.

107. The method of aspect 106, wherein the method does not comprise a purification step between the method of any one of aspects 88 to 105 and the method of any one of aspects 60 to 84.

108. The method of any one of aspects 52, or 62 to 84, wherein the polysaccharide or the GAC polysaccharide is the one or more polysaccharide as defined in any one of aspects 18 to 23 or 26 to 32.

109. The method of aspect 108, wherein the polysaccharide is a GAC polysaccharide.

110. The method of any one of aspects 52, 85 to 108, wherein the oxidised polysaccharide or the oxidised GAC polysaccharide is an oxidised version of the one or more polysaccharide as defined in any one of aspects 18 to 23 or 26 to 32.

111. The method of aspect 110, wherein the polysaccharide is an oxidised GAC polysaccharide.

112. The method of any one of aspects 62 to 110, wherein the carrier polypeptide/protein is a carrier polypeptide/protein as defined in any one of aspects 2 to 17.

113. The method of any one of aspects 60 to 111, wherein the carrier polypeptide/protein comprises:
(i) an amino acid sequence of any one of SEQ ID NO: 1-7;
(ii) an amino acid sequence at least 90%, at least 95%, at least 98%, at least 99% or 100% identical to any one of SEQ ID NO: 1-7; or
(iii) amino acid sequence at least 95% identical to a fragment of at least 500 amino acids of any one of SEQ ID NO: 1-7.

114. The method of any one of aspects 60 to 113, wherein the method provides batch-to-batch consistency.

115. A polysaccharide conjugate obtainable by the method of any one of aspects 60 to 114.

116. A polysaccharide conjugate obtained by the method of any one of aspects 60 to 114.

117. The polysaccharide conjugate of aspect 115 or 116 for use in medicine.

118. The polysaccharide conjugate of aspect 115 or 116 for use in raising an immune response in a mammal, for example, for treating and/or preventing one or more disease.

119. The polysaccharide conjugate of aspect 115 or 116 for use in treating and/or preventing GAS infection.

120. Use of a polysaccharide conjugate of aspect 115 or 116 for raising an immune response in a mammal, for example, for treating and/or preventing one or more disease.

121. Use of a polysaccharide conjugate of aspect 115 or 116 for treating and/or preventing GAS infection.

122. Use of a polysaccharide conjugate of aspect 115 or 116 for the manufacture of a medicament for raising an immune response in a mammal, for example, for treating and/or preventing one or more disease.

123. Use of a polysaccharide conjugate of aspect 115 or 116 for the manufacture of a medicament for treating and/or preventing GAS infection.

124. A method of raising an immune response in a mammal, for example, for treating and/or preventing one or more disease, the method comprising administering to a mammal an effective amount of a polysaccharide conjugate of any one of aspects 1-37, 115 or 116, the composition of aspect 38, the immunogenic composition of aspect 39, a vaccine of any one of aspects 40-42.

125. A method of treating and/or preventing one or more disease, the method comprising administering to a mammal an effective amount of a polysaccharide conjugate of any one of aspects 1-37, 115 or 116, the composition of aspect 38, the immunogenic composition of aspect 39, a vaccine of any one of aspects 40-42.

126. The method of aspect 124 or 125, wherein one of the one more disease is GAS infection.

1. A method of oxidising a polysaccharide comprising a step of oxidisation of the polysaccharide comprising the steps of:
I. oxidisation of the polysaccharide by reacting the polysaccharide with
i. an oxidising agent,
ii. in a suitable buffer,
iii. at a suitable temperature,
iv. for a suitable time.

2. A method of oxidising polysaccharide comprising the steps of:
I. oxidisation of polysaccharide by reacting:
i. polysaccharide, for example, at a concentration of 0.1-100 mg/ml, e.g., 0.5-50, 0.5-25, 1-10, 2.5-7.5, 4-6 or 5 mg/mL, with
ii. oxidising agent (for example, NaIO₄ [sodium periodate+, KMnO₄ [potassium permanganate], periodic acid [HIO₄], or lead tetra-acetate [Pb(OAc)₄]), at a concentration 0.5-10M,
iii. in a suitable buffer (for example, 200 mM phosphate buffer, or borate buffer) pH 3-9, for example, pH 5-8 (for example, pH5 or pH 8),
iv. at a suitable temperature (for example, 20-30° C., such as 25° C.),
v. for a suitable time (for example, 15 min-5 hr, such as, 30 min-3 hr, 30 min-1 hr, or 30 mins);
II. (optionally) quenching of residual NaIO₄ by:
vi. addition of a suitable amount of reducing agent, for example, Na₂SO₃ (sodium sulfite), for example, at a molar excess with respect to the concentration of NaIO₄ in step I(ii), for example, 5-10 times the concentration of NaIO₄ in step I(ii), or 16 mM,
vii. at a suitable temperature (e.g., 20-30° C., room temperature, or 25° C.),
viii. for a suitable time (e.g., 10-30 min, or 15 min);
III. (optionally) purification and/or concentration of oxidised polysaccharide, for example, using a method selected from the group consisting of lyophilisation, centrifugal evaporation, rotary evaporation, and tangential flow filtration.

3. The method of claim 1 or 2, wherein at least one of the polysaccharide concentration, the oxidising agent, the oxidising agent concentration, the suitable buffer, the suitable temperature and the suitable time used ensure that the method achieves at least 5%, at least 10%, at least 15%, between 10% and 30%, between 10% and 25%, or around 15% oxidation of the polysaccharide.

4. The method of any one of claims 1 to 3, wherein the polysaccharide is GAC and at least one of the polysaccharide concentration, the oxidising agent, the oxidising agent concentration, the suitable buffer, the suitable temperature and the suitable time used in the method ensures that the method achieves a GAC recovery of at least 60%, at least 65%, at least 70%, at least 75%, between 60% and 100%, between 65% and 100%, between 70% and 90%, or between 75% and 90%.

5. A method of conjugating oxidised polysaccharide comprising a step of reacting:
a. oxidised polysaccharide with;
b. a carrier polypeptide/protein; and
c. sodium cyanoborohydride;
d. in borate buffer;
e. at a suitable temperature;
f. for a suitable time.

6. A method of conjugating oxidised polysaccharide comprising the steps of:
A. reacting:
a. oxidised polysaccharide at a concentration of 5-75 mg/mL (for example, 40 mg/mL) with;
b. protein at a concentration of 5-75 mg/mL (for example 40 mg/mL); and
c. NaBH₃CN (sodium cyanoborohydride) concentration of 0.5-10.0 mg/ml;
d. In borate buffer pH 7-9, for example, pH 7.5-8.5, pH8;
e. at a suitable temperature (for example, 17.5-42.5° C., room temperature, 25° C., 30° C. or 37° C.),
f. for a suitable time (e.g., 1 hr, 2 hr, 4 hr, 6 hr, 0.5 to 3 days, 1 day or 2 days;
B. (optionally) quenching of residual aldehydes of oxidised polysaccharide by:
j. addition of a suitable amount of NaBH₄ (e.g., an NaBH₄:polysaccharide ratio [w/w] of 0.5:1, or, for example, at a molar excess with respect to the aldehyde groups generated or moles of oxidized polysaccharide, for example, 5-10 times, 50 times, 100 times or 1000 times),
k. at a suitable temperature (e.g., 20-30° C., 25° C., or room temperature),
l. for a suitable time (e.g., 1 to 12 hr, 2-4 hr).
C. (optionally) purification of the polysaccharide conjugate resulting from step (B) by tangential flow filtration (TFF) and/or sterile filtration (e.g., TFF followed by sterile filtration).

7. The method of claim 5 or 6, wherein at least one of the oxidised polysaccharide concentration, the carrier polypeptide/protein concentration, the sodium cyanoborohydride concentration, the pH of the borate buffer, and the suitable temperature used in the method ensures that the method achieves a polysaccharide to carrier polypeptide/protein ratio of at least 0.25, at least 0.3, at least 0.35, at least 0.4, between 0.25 and 1, between 0.3 and 0.8, or between 0.4 and 0.8.

8. The method of any one of claims 5 to 7, wherein the polysaccharide is GAC and at least one of the oxidised polysaccharide concentration, the carrier polypeptide/protein concentration, the sodium cyanoborohydride concentration, the pH of the borate buffer, and the suitable temperature used in the method ensures that the method achieves a GAC recovery of at least 25%, at least 30%, at least 35%, between 25% and 80%, between 30% and 70%, or between 35% and 60%.

9. The method of any one of claims 5 to 8, wherein the ratio of polysaccharide to carrier polypeptide/protein to sodium cyanoborohydride is 1-20:1-20:1 mg/ml, 5-15:5-15:1 mg/ml, or around 8:8:1 w/w/v.

10. A method of conjugating polysaccharide to carrier polypeptide/protein comprising the method of any one of claims 1 to 4, followed by the method of any one of claims 5 to 9.

11. The method of any one of claims 1 to 10, wherein the polysaccharide is a microbial polysaccharide such as a bacterial polysaccharide, an archaeal polysaccharide, a fungal polysaccharide, or a protist polysaccharide.

12. The method of any one of claims 1 to 11, wherein the polysaccharide is a GAC polysaccharide.

13. The method of any one of claims 5 to 12, wherein the oxidised polysaccharide is an oxidised version of the polysaccharide of claim 11 or 12.

14. The method of any one of claims 1 to 13, wherein the carrier polypeptide/protein comprises:
(i) an amino acid sequence of any one of SEQ ID NO: 1-7;
(ii) an amino acid sequence at least 90%, at least 95%, at least 98%, at least 99% or 100% identical to any one of SEQ ID NO: 1-7; or
(iii) amino acid sequence at least 95% identical to a fragment of at least 500 amino acids of any one of SEQ ID NO: 1-7.

15. A polysaccharide conjugate produced according to the method of any one of claims 5 to 14.

16. A polysaccharide conjugate obtainable by the method of any one of claims 5 to 14.

17. A polysaccharide conjugate comprising or consisting of one or more polysaccharide conjugated to a carrier polypeptide, wherein the carrier polypeptide comprises a polypeptide:
(a) selected from the group consisting of a Streptococcus pyogenes SpyAD, a Streptococcus pyogenes SpyCEP, and a Streptococcus pyogenes SLO; or
(b) CRM₁₉₇; or
(c) a variant, fragment and/or fusion of (a) or (b).

18. The polysaccharide conjugate of any one of claims 15 to 17, wherein the carrier polypeptide is:
(a) a Streptococcus pyogenes SpyAD (Spy0269); or
(b) a variant, fragment and/or fusion of a Streptococcus pyogenes SpyAD (Spy0269).

19. The polysaccharide conjugate of any one of claims 15 to 18, wherein the carrier polypeptide comprises or consists of:
(i) an amino acid sequence of SEQ ID NO: 1 or SEQ ID NO: 2;
(ii) an amino acid sequence that varies from SEQ ID NO: 1 or SEQ ID NO: 2 by from 1 to 10 single amino acid alterations;
(iii) an amino acid sequence with at least 70%, 80%, 85%, 90%, 95%, 96%, 97%, 98%, 99% or at least 99.5% sequence identity with SEQ ID NO: 1 or SEQ ID NO: 2; and/or
(iv) a fragment of at least 10 consecutive amino acids from SEQ ID NO: 1 or SEQ ID NO: 2, for example, at least 20, 30, 40, 50, 60, 70, 80, 90, 100, 125, 150, 175, 200, 250, 275, 280, 290, 300, 310, 320, 330, 340, or 350 consecutive amino acids from SEQ ID NO: 1 or SEQ ID NO: 2.

20. The polysaccharide conjugate of any one of claims 15 to 19, wherein the carrier polypeptide comprises or consists of an amino acid having at least 95% identity with a fragment of at least 300 amino acids of SEQ ID NO: 1 or SEQ ID NO: 2.

21. The polysaccharide conjugate of any one of claims 15 to 20, wherein the carrier polypeptide comprises or consist of an amino acid having at least 95% identity with SEQ ID NO: 1 or SEQ ID NO: 2.

22. A method of:
(i) raising an immune response in a mammal, for example, for treating and/or preventing one or more disease; and/or
(ii) treating and/or preventing GAS infection,
the method comprising administering to a mammal an effective amount of a polysaccharide conjugate of any one of claim 15 to 22.

23. The polysaccharide conjugate of any one of claims 15 to 22 for use in:
(i) medicine;
(ii) raising an immune response in a mammal, for example, for treating and/or preventing one or more disease; and/or
(iii) treating and/or preventing GAS infection.

24. Use of a polysaccharide conjugate of any one of claims 15 to 22 for:
(i) raising an immune response in a mammal, for example, for treating and/or preventing one or more disease; and/or
(ii) treating and/or preventing GAS infection.

25. Use of a polysaccharide conjugate of any one of claims 15 to 22 for the manufacture of a medicament for:
(i) raising an immune response in a mammal, for example, for treating and/or preventing one or more disease; and/or
(ii) treating and/or preventing GAS infection.


SEQUENCE LISTING (7 sequences)

SEQ ID NO: 1 - protein, 824 aa, Streptococcus pyogenes
MSVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 2 - protein, 873 aa, Streptococcus pyogenes
MDLEQTKPNQVKQKIALTSTIALLSASVGVSHQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQRALKAGVVMLAAVGLTGFRFRKESK

SEQ ID NO: 3 - protein, 1581 aa, Streptococcus pyogenes
MADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIATGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTAMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKA

SEQ ID NO: 4 - protein, 1647 aa, Streptococcus pyogenes
MEKKQRFSLRKYKSGTFSVLIGSVFLVMTTTVAADELSTMSEPTITNHAQQQAQHLTNTELSSAESKSQDTSQITLKTNREKEQSQDLVSEPTTTELADTDAASMANTGSDATQKSASLPPVNTDVHDWVKTKGAWDKGYKGQGKVVAVIDTGIDPAHQSMRISDVSTAKVKSKEDMLARQKAAGINYGSWINDKVVFAHNYVENSDNIKENQFEDFDEDWENFEFDAEAEPKAIKKHKIYRPQSTQAPKETVIKTEETDGSHDIDWTQTDDDTKYESHGMHVTGIVAGNSKEAAATGERFLGIAPEAQVMFMRVFANDIMGSAESLFIKAIEDAVALGADVINLSLGTANGAQLSGSKPLMEAIEKAKKAGVSVVVAAGNERVYGSDHDDPLATNPDYGLVGSPSTGRTPTSVAAINSKWVIQRLMTVKELENRADLNHGKAIYSESVDFKDIKDSLGYDKSHQFAYVKESTDAGYNAQDVKGKIALIERDPNKTYDEMIALAKKHGALGVLIFNNKPGQSNRSMRLTANGMGIPSAFISHEFGKAMSQLNGNGTGSLEFDSVVSKAPSQKGNEMNHFSNWGLTSDGYLKPDITAPGGDIYSTYNDNHYGSQTGTSMASPQIAGASLLVKQYLEKTQPNLPKEKIADIVKNLLMSNAQIHVNPETKTTTSPRQQGAGLLNIDGAVTSGLYVTGKDNYGSISLGNITDTMTFDVTVHNLSNKDKTLRYDTELLTDHVDPQKGRFTLTSHSLKTYQGGEVTVPANGKVTVRVTMDVSQFTKELTKQMPNGYYLEGFVRFRDSQDDQLNRVNIPFVGFKGQFENLAVAEESIYRLKSQGKTGFYFDESGPKDDIYVGKHFTGLVTLGSETNVSTKTISDNGLHTLGTFKNADGKFILEKNAQGNPVLAISPNGDNNQDFAAFKGVFLRKYQGLKASVYHASDKEHKNPLWVSPESFKGDKNFNSDIRFAKSTTLLGTAFSGKSLTGAELPDGHYHYVVSYYPDVVGAKRQEMTFDMILDRQKPVLSQATFDPETNRFKPEPLKDRGLAGVRKDSVFYLERKDNKPYTVTINDSYKYVSVEDNKTFVERQADGSFILPLDKAKLGDFYYMVEDFAGNVAIAKLGDHLPQTLGKTPIKLKLTDGNYQTKETLKDNLEMTQSDTGLVTNQAQLAVVHRNQPQSQLTKMNQDFFISPNEDGNKDFVAFKGLKNNVYNDLTVNVYAKDDHQKQTPIWSSQAGASVSAIESTAWYGITARGSKVMPGDYQYVVTYRDEHGKEHQKQYTISVNDKKPMITQGRFDTINGVDHFTPDKTKALDSSGIVREEVFYLAKKNGRKFDVTEGKDGITVSDNKVYIPKNPDGSYTISKRDGVTLSDYYYLVEDRAGNVSFATLRDLKAVGKDKAVVNFGLDLPVPEDKQIVNFTYLVRDADGKPIENLEYYNNSGNSLILPYGKYTVELLTYDTNAAKLESDKIVSFTLSADNNFQQVTFKITMLATSQITAHFDHLLPEGSRVSLKTAQDQLIPLEQSLYVPKAYGKTVQEGTYEVVVSLPKGYRIEGNTKVNTLPNEVHELSLRLVKVGDASDSTGDHKVMSKNNSQALTASATPTKSTTSATAKALPSTGEKMGLKLRIVGLVLLGLTCVFSRKKSTKD

SEQ ID NO: 5 - protein, 543 aa, Streptococcus pyogenes
MASESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNLAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAFEWWRKVIDERDVKLSKEINVNISGSTLSPYGSITYK

SEQ ID NO: 6 - protein, 571 aa, Streptococcus pyogenes
MSNKKTFKKYSRVAGLLTAALIIGNLVTANAESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 7 - protein, 536 aa, Artificial Sequence
  Non-toxic mutant of diphtheria toxin
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS